(19) United States
(12) Patent Application Publication
(10) Pub. No.: US 2026/0131107 A1
(43) Pub. Date: May 14, 2026

(54) CATHETERS

(71) Applicant: ConvaTec Limited, Flintshire (GB)

(72) Inventors: David Pollard, Merseyside (GB); Rachel Pytel, Newton, MA (US); Daniel Metcalf, Greater Manchester (GB); Kate Meredith, Flintshire (GB); Daniel Allen, Staffordshire (GB)

(21) Appl. No.: 19/343,798

(22) Filed: Sep. 29, 2025

Related U.S. Application Data
(63) Continuation of application No. PCT/GB2024/050891, filed on Mar. 28, 2024.
(60) Provisional application No. 63/455,315, filed on Mar. 29, 2023.

(30) Foreign Application Priority Data
May 24, 2023 (GB) 2307755.5

(51) Int. Cl. A61M 25/00; A61L 2/18; A61L 103/15

(52) U.S. Cl. CPC A61M 25/002; A61L 2/18; A61L 2103/15; A61L 2400/10; A61M 2202/0014; A61M 2202/0496; A61M 2205/0205; A61M 2205/0222; A61M 2209/06

(57) ABSTRACT

A packaged intermittent urinary catheter comprising a single use catheter, a packaging surrounding the catheter, and a fluid reservoir holding a lubricant which, upon rupturing the fluid reservoir, is transferred into the packaging to immerse at least a portion of the catheter in the lubricant to prepare the catheter for use, wherein the lubricant comprises an antimicrobial medium. The invention further provides a method of using a packaged intermittent urinary catheter, and the use of a medium comprising at least one species independently chosen from: hypochlorous acid, at least one hypochlorite salt, chlorine dioxide, at least one chlorhexidine salt, and combinations thereof, as an antimicrobial medium for a single use intermittent urinary catheter.

### TECHNICAL FIELD OF THE INVENTION

The present invention relates to packaged intermittent urinary catheters comprising a lubricant.

### BACKGROUND TO THE INVENTION

Urinary catheterisation is a process involving insertion of a catheter through an individual's urethra and into their bladder, where it is retained to empty the bladder of urine. There are two major types of urinary catheterisation-intermittent catheterisation and long-term catheterisation. Intermittent urinary catheterisation involves retaining the catheter in the bladder for only the time period required for emptying, after which the catheter is removed. The process differs from long-term catheterisation, which makes use of an indwelling or Foley catheter that is inserted into the bladder for long periods of time (several days to months) to discharge the residual urine of the bladder continuously throughout the day.

Catheterisation is often usd by patients suffering from abnormalities of the urinary system, resulting in urinary incontinence and/or a lack of control in permitting voluntary urination. Such individuals would typically make use of intermittent catheters several times a day.

Catheters are useful devices, providing users with independence and freedom to self-catheterise as and when required, without having to rely on trained personnel to be present. This, however, increases the need for catheters to be user friendly: in particular, both easy to insert and remove with minimum discomfort caused, and safe to use with features for minimising risk of infection. Users often report experiencing pain and discomfort upon insertion and/or removal of catheters. Users have, for instance, reported experiencing bladder spasms, burning sensations, and bleeding.

It is also easy for catheters to become contaminated and for bacteria to be introduced into the urethra and along the urinary tract. As a result, urinary tract infections (UTI) are common in individuals who practice self-catheterisation.

There are two major types of urinary catheter—reusable catheters and singleuse catheters. Each are typically packaged prior to us.

Packaged reusable catheters typically include an envelope comprising a catheter and a sealed rupturable container holding an antimicrobial sterilising medium, in addition to a container comprising a lubricant for lubricating the catheter prior to use. Upon first use of the catheter, the reusable catheter is removed from the envelope, which is retained for re-use, and the catheter is at least partially coated with lubricant and then used. Prior to second use of the catheter, the container holding the antimicrobial sterilising medium is ruptured causing the medium to be transferred into the envelope. The once-used catheter is then inserted into the envelope and is immersed in the antimicrobial sterilising medium to sterilise the catheter before re-use.

Packaged single use catheters typically comprise a container comprising a water or saline-based lubricant for lubricating the catheter prior to use. Catheters, in particular single use catheters, are generally manufactured under sterile conditions and supplied in a sterile package. As such, there is no expectation that there is a need to sterilise an intermittent single use catheter prior to use.

Patients who use catheterisation often develop urinary tract infections (UTIs). UTIs may be caused, at least in part, by movement of bacteria (for example Escherichia coli and/or Enterococcus faecal is} from the distal urethra (meatus) (sometimes known as ‘urethral’), which is typically colonised by bacteria, along the urethra toward the bladder during insertion of an intermittent catheter. Escherichia coli is attributed to around 80% of UTIs. With regular insertions of intermittent catheters, the described movement of bacteria is repeated frequently, therefore, increasing the likelihood that bacteria will colonise along the urethra and reach the bladder. This, in turn, increases the possibility that the patient will develop a UTI.

Immersion of a reusable catheter in an antimicrobial sterilising medium prior to re-use eliminates, or at least eliminates a significant portion of, bacteria on the catheter before it is re-used. However, there still remains a problem of the movement of bacteria from the distal urethra (meatus), along the urethra toward the bladder during insertion of an intermittent catheter and, therefore, the colonisation of bacteria along the urethra.

This problem is particularly found with patients who use single-use catheters, despite the single-use catheter being manufactured and packaged in a sterile environment.

Moreover, some sterilisation mediums of the prior art are known to adversely affect catheter lubricity. Disadvantageously, this increases discomfort for the user when inserting the catheter as the low friction property of the catheter is at least partially negated by the sterilisation medium. In more serious cases, this can cause incidences of urethral trauma which can significantly affects a user's physical and mental health, and their ability to self-catheterise.

It is therefore an aim of embodiments of the present invention to overcome or mitigate at least one problem of the prior art, whether expressly described herein or not.

### SUMMARY OF THE INVENTION

In broad terms, the present invention concerns a packaged intermittent urinary catheter. The packaged intermittent urinary catheter may comprise a catheter. The packaged intermittent urinary catheter may comprise a packaging surrounding the catheter. The packaged intermittent urinary catheter may comprise a fluid reservoir holding a lubricant. Upon rupturing the fluid reservoir, the lubricant may be transferred into the packaging to immerse at least a portion of the catheter in the lubricant to prepare the catheter for use. The lubricant may comprise an antimicrobial medium.

According to a broad aspect of the present invention, there is provided a packaged intermittent urinary catheter comprising:
a catheter;
a packaging surrounding the catheter; and
a fluid reservoir holding a lubricant,
wherein the lubricant comprises an antimicrobial medium.

Upon rupturing the fluid reservoir, the lubricant may be transferred into the packaging to immerse at least a portion of the catheter in the lubricant to prepare the catheter for use

The catheter may be a single use catheter.

The packaging may be formed of flexible liquid impermeable material. Beneficially, the flexible liquid impermeable material may provide a sterile environment for the catheter prior to use.

The packaging may comprise means to form an opening. The opening may be at at least one end of the packaging. Advantageously, the opening may allow the catheter to be removed from the packaging upon use.

The fluid reservoir may be formed of a flexible liquid impermeable material. Beneficially, the flexible liquid impermeable material may provide a sealed, rupturable container which can be ruptured with relative ease by a user, in particular a user with limited dexterity.

As such, a first aspect of the invention provides a packaged intermittent urinary catheter comprising: a single use catheter; a packaging surrounding the catheter, the packaging being formed of flexible liquid impermeable material which provides a sterile environment for the catheter prior to use, wherein the packaging comprises means to form an opening at at least one end of the packaging through which the catheter is removed upon use; and a fluid reservoir formed of a flexible liquid impermeable material providing a sealed, rupturable container holding a lubricant which, upon rupturing the fluid reservoir, is transferred into the packaging to immerse at least a portion of the catheter in the lubricant to prepare the catheter for use, wherein the lubricant comprises an antimicrobial medium.

Conventional single use catheters are used with a saline or water-based lubricant which provides no, or negligible, antimicrobial effect in use. As such, despite being packaged in a sterile environment, a conventional single use catheter provides no antimicrobial effect upon the user when in use. Thus, during insertion of a conventional single use intermittent catheter, bacteria (for example Escherichia coli and/or Enterococcus faecalis) is moved from the distal urethra (meatus) along the urethra toward the bladder, therefore facilitating colonisation of bacteria along the urethra and increasing the possibility that the patient will develop a UTI.

Advantageously, the present invention provides an intermittent catheter which, prior to use, is at least partially coated in a lubricant comprising an antimicrobial medium. As such, the lubricant of the present invention lowers friction, therefore causing reduced pain and/or reduced discomfort for the user upon insertion which reduces incidence of urethral trauma, while simultaneously providing an antimicrobial effect in use.

Moreover, the antimicrobial medium provides an antimicrobial effect whereby, in use, the lubricant contacts a user's distal urethra (meatus) and urethra. This means that the bacteria (for example Escherichia coli and/or Enterococcus faecalis) which typically colonise the distal urethra (meatus) are eliminated when the catheter is inserted into the urethra of a user. As such, a significant number of bacteria are eliminated at their location of colonisation at the distal urethra (meatus) and, therefore, movement of bacteria along the urethra toward the bladder is significantly decreased. Further, as the catheter is inserted along the urethra, the lubricant comprising the antimicrobial medium eliminates, or at least significantly reduces the number of, bacteria which are located along the urethra in addition to bacteria located at the distal urethra (meatus).

As such, advantageously, the present invention significantly minimises the movement of bacteria from the distal urethra (meatus), along the urethra, toward the bladder. It follows, therefore, that the possibility of the user developing a UTI is considerably reduced.

The lubricant comprising such antimicrobial medium allows for effective elimination of bacteria in the user's urinary tract and simultaneous lubrication of the catheter, which can be achieved by simply contacting the catheter with the lubricant.

By ‘single-use catheter’, we mean that a user will remove the catheter from a package, use the catheter once, and then dispose of the catheter and package.

The antimicrobial medium may comprise an aqueous chlorine solution.

Beneficially, aqueous solutions are particularly effective as water allows for optimal catheter surface lubricity.

The aqueous chlorine solution may comprise at least one chlorine-containing species independently chosen from: hypochlorous acid, at least one hypochlorite salt, chlorine dioxide, at least one chlorhexidine salt, and combinations thereof.

The aqueous chlorine solution may comprise at least one chlorine-containing species independently chosen from: hypochlorous acid, at least one hypochlorite salt, chlorine dioxide, and combinations thereof.

The at least one chlorine-containing species may be hypochlorous acid.

Embodiments comprising hypochlorous acid as the at least one chlorine-containing species are particularly advantageous because hypochlorous acid rapidly degrades into harmless salt and water following its use as an antimicrobial medium. Moreover, hypochlorous acid rapidly kills bacteria, for example E. coli, K. pneumoniae, E. faecalis, P. mirabilis, and P. aeruginosa. Specifically, hypochlorous acid reduces bacterial numbers of more than a 4 log reduction. Further advantageously, hypochlorous acid is a highly effective antimicrobial which rapidly kills bacteria before degrading into harmless salt and water. As such, hypochlorous acid does not accumulate within a patient's body when repeatedly used as an antimicrobial medium in a lubricant in which a catheter is at least partly immersed prior to use by the patient.

Further advantageously, use of such an aqueous chlorine solution causes minimal change to the relevant surface chemistry of the catheter upon contact, and so allows for the catheter to be used safely and for relatively long periods, as necessary, without adversely impacting catheter lubricity and without requiring re-lubrication.

In some embodiments, there is provided a packaged intermittent urinary catheter comprising: a single use catheter; a packaging surrounding the catheter; and a fluid reservoir holding a lubricant which, upon rupturing the fluid reservoir, is transferred into the packaging to immerse at least a portion of the catheter in the lubricant to prepare the catheter for use, wherein the lubricant comprises an antimicrobial medium, further wherein the antimicrobial medium comprises an aqueous chlorine solution comprising at least one chlorine-containing species independently chosen from: hypochlorous acid, at least one hypochlorite salt, chlorine dioxide, and combinations thereof.

In some embodiments, there is provided a packaged intermittent urinary catheter comprising: a single use catheter; a packaging surrounding the catheter; and a fluid reservoir holding a lubricant which, upon rupturing the fluid reservoir, is transferred into the packaging to immerse at least a portion of the catheter in the lubricant to prepare the catheter for use, wherein the lubricant comprises an antimicrobial medium, further wherein the antimicrobial medium comprises an aqueous chlorine solution comprising at least one chlorine-containing species, wherein the at least one chlorine-containing species is hypochlorous acid.

In embodiments comprising at least one chlorhexidine salt, the at least one chlorhexidine salt may be chlorhexidine digluconate, chlorhexidine diacetate, or combinations thereof. In some preferred embodiments, the at least one chlorhexidine salt is chlorhexidine digluconate.

The at least one chlorine-containing species may be present in an amount of between about 100 and about 4500 ppm, about 150 and about 4400 ppm, about 200 and about 4300 ppm, about 250 and about 4200 ppm, about 300 and about 4100 ppm, about 350 and about 4000 ppm, about 400 and about 3900 ppm, about 450 and about 3800 ppm, about 500 and about 3700 ppm, about 550 and about 3600 ppm, about 600 and about 3500 ppm, about 650 and about 3400 ppm, about 700 and about 3300 ppm, about 750 and about 3200 ppm, about 800 and about 3100 ppm, about 850 and about 3000 ppm, about 900 and about 2900 ppm, about 950 and about 2800 ppm, about 1000 and about 2700 ppm, about 1050 and about 2600 ppm, about 1100 and about 2500 ppm, about 1150 and about 2400 ppm, about 1200 and about 2300 ppm, about 1250 and about 2200 ppm, about 1300 and about 2100 ppm, about 1350 and about 2000 ppm, about 1400 and about 1900 ppm, about 1450 and about 1800 ppm, about 1500 and about 1700 ppm, or about 1600 ppm, of the overall aqueous content of the fluid reservoir.

The at least one chlorine-containing species may be present in an amount of about 100 ppm, about 150 ppm, about 200 ppm, about 250 ppm, about 300 ppm, about 350 ppm, about 400 ppm, about 450 ppm, about 500 ppm, about 550 ppm, about 600 ppm, about 650 ppm, about 700 ppm, about 750 ppm, about 800 ppm, about 850 ppm, about 900 ppm, about 950 ppm, about 1000 ppm, about 1050 ppm, about 1100 ppm, about 1150 ppm, about 1200 ppm, about 1250 ppm, about 1300 ppm, about 1350 ppm,
about 1400 ppm, about 1450 ppm, about 1500 ppm, about 1550 ppm, about 1600 ppm, about 1650 ppm, about 1700 ppm, about 1750 ppm, about 1800 ppm, about 1850 ppm, about 1900 ppm, about 1950 ppm, about 2000 ppm, about 2050 ppm, about 2100 ppm, about 2150 ppm, about 2200 ppm, about 2250 ppm, about 2300 ppm, about 2350 ppm, about 2400 ppm, about 2450 ppm, about 2500 ppm, about 2550 ppm, about 2600 ppm, about 2650 ppm, about 2700 ppm, about 2750 ppm, about 2800 ppm, about 2850 ppm, about 2900 ppm, about 2950 ppm, about 3000 ppm, about 3050 ppm, about 3100 ppm, about 3150 ppm, about 3200 ppm, about 3250 ppm, about 3300 ppm, about 3350 ppm, about 3400 ppm, about 3450 ppm, about 3500 ppm, about 3550 ppm, about 3600 ppm, about 3650 ppm, about 3700 ppm, about 3750 ppm, about 3800 ppm, about 3850 ppm, about 3900 ppm, about 3950 ppm, about 4000 ppm, about 4050 ppm, about 4100 ppm, about 4150 ppm, about 4200 ppm, about 4250 ppm, about 4300 ppm, about 4350 ppm, about 4400 ppm, about 4450 ppm, or about 4500 ppm, of the overall aqueous content of the fluid reservoir.

The at least one chlorine-containing species may be present in an amount of at least about 100 ppm, about 150 ppm, about 200 ppm, about 250 ppm, about 300 ppm, about 350 ppm, about 400 ppm, about 450 ppm, about 500 ppm, about 550 ppm, about 600 ppm, about 650 ppm, about 700 ppm, about 750 ppm, about 800 ppm, about 850 ppm, about 900 ppm, about 950 ppm, about 1000 ppm, about 1050 ppm, about 1100 ppm, about 1150 ppm, about 1200 ppm, about 1250 ppm, about 1300 ppm, about 1350 ppm, about 1400 ppm, about 1450 ppm, about 1500 ppm, about 1550 ppm, about 1600 ppm, about 1650 ppm, about 1700 ppm, about 1750 ppm, about 1800 ppm, about 1850 ppm, about 1900 ppm, about 1950 ppm, about 2000 ppm, about 2050 ppm, about 2100 ppm, about 2150 ppm, about 2200 ppm, about 2250 ppm, about 2300 ppm, about 2350 ppm, about 2400 ppm, about 2450 ppm, about 2500 ppm, about 2550 ppm, about 2600 ppm, about 2650 ppm, about 2700 ppm, about 2750 ppm, about 2800 ppm, about 2850 ppm, about 2900 ppm, about 2950 ppm, about 3000 ppm, about 3050 ppm, about 3100 ppm, about 3150 ppm, about 3200 ppm, about 3250 ppm, about 3300 ppm, about 3350 ppm, about 3400 ppm, about 3450 ppm, about 3500 ppm, about 3550 ppm, about 3600 ppm, about 3650 ppm, about 3700 ppm, about 3750 ppm, about 3800 ppm, about 3850 ppm, about 3900 ppm, about 3950 ppm, about 4000 ppm, about 4050 ppm, about 4100 ppm, about 4150 ppm, about 4200 ppm, about 4250 ppm, about 4300 ppm, about 4350 ppm, about 4400 ppm, about 4450 ppm, or at least about 4500 ppm, of the overall aqueous content of the fluid reservoir.

The at least one chlorine-containing species may be present in an amount of no more than about 100 ppm, about 150 ppm, about 200 ppm, about 250 ppm, about 300 ppm, about 350 ppm, about 400 ppm, about 450 ppm, about 500 ppm, about 550 ppm, about 600 ppm, about 650 ppm, about 700 ppm, about 750 ppm, about 800 ppm, about 850 ppm, about 900 ppm, about 950 ppm, about 1000 ppm, about 1050 ppm, about 1100 ppm, about 1150 ppm, about 1200 ppm, about 1250 ppm, about 1300 ppm, about 1350 ppm, about 1400 ppm, about 1450 ppm, about 1500 ppm, about 1550 ppm, about 1600 ppm, about 1650 ppm, about 1700 ppm, about 1750 ppm, about 1800 ppm, about 1850 ppm, about 1900 ppm, about 1950 ppm, about 2000 ppm, about 2050 ppm, about 2100 ppm, about 2150 ppm, about 2200 ppm, about 2250 ppm, about 2300 ppm, about 2350 ppm, about 2400 ppm, about 2450 ppm, about 2500 ppm, about 2550 ppm, about 2600 ppm, about 2650 ppm, about 2700 ppm, about 2750 ppm, about 2800 ppm, about 2850 ppm, about 2900 ppm, about 2950 ppm, about 3000 ppm, about 3050 ppm, about 3100 ppm, about 3150 ppm, about 3200 ppm, about 3250 ppm, about 3300 ppm, about 3350 ppm, about 3400 ppm, about 3450 ppm, about 3500 ppm, about 3550 ppm, about 3600 ppm, about 3650 ppm, about 3700 ppm, about 3750 ppm, about 3800 ppm, about 3850 ppm, about 3900 ppm, about 3950 ppm, about 4000 ppm, about 4050 ppm, about 4100 ppm, about 4150 ppm, about 4200 ppm, about 4250 ppm, about 4300 ppm, about 4350 ppm, about 4400 ppm, about 4450 ppm, or no more than about 4500 ppm, of the overall aqueous content of the fluid reservoir.

The at least one chlorine-containing species may be present in an amount of from about 50 to about 4500 ppm, from about 50 to about 4250 ppm, from about 50 to about 4000 ppm, from about 50 to about 3750 ppm, from about 50 to about 3500 ppm, from about 50 to about 3250 ppm, from about 50 to about 3000 ppm, from about 50 to about 2750 ppm, from about 50 to about 2500 ppm, from about 50 to about 2250 ppm, from about 50 to about 2000 ppm, from about 50 to about 1750 ppm, from about 50 to about 1500 ppm, from about 50 to about 1250 ppm, from about 50 to about 1000 ppm, from about 50 to about 900 ppm, from about 50 to about 800 ppm, from about 50 to about 700 ppm, from about 50 to about 600 ppm, from about 50 to about 500 ppm, from about 50 to about 400 ppm, from about 50 to about 300 ppm, from about 50 to about 200 ppm, or from about 50 to about 100 ppm, of the overall aqueous content of the fluid reservoir.

The at least one chlorine-containing species may be present in an amount of from about 100 to about 4500 ppm, from about 100 to about 4250 ppm, from about 100 to about 4000 ppm, from about 100 to about 3750 ppm, from about 100 to about 3500 ppm, from about 100 to about 3250 ppm, from about 100 to about 3000 ppm, from about 100 to about 2750 ppm, from about 100 to about 2500 ppm, from about 100 to about 2250 ppm, from about 100 to about 2000 ppm, from about 100 to about 1750 ppm, from about 100 to about 1500 ppm, from about 100 to about 1250 ppm, from about 100 to about 1000 ppm, from about 100 to about 900 ppm, from about 100 to about 800 ppm, from about 100 to about 700 ppm, from about 100 to about 600 ppm, from about 100 to about 500 ppm, from about 100 to about 400 ppm, from about 100 to about 300 ppm, or from about 100 to about 200 ppm, of the overall aqueous content of the fluid reservoir.

The at least one chlorine-containing species may be present in an amount of from about 200 to about 4500 ppm, from about 200 to about 4250 ppm, from about 200 to about 4000 ppm, from about 200 to about 3750 ppm, from about 200 to about 3500 ppm, from about 200 to about 3250 ppm, from about 200 to about 3000 ppm, from about 200 to about 2750 ppm, from about 200 to about 2500 ppm, from about 200 to about 2250 ppm, from about 200 to about 2000 ppm, from about 200 to about 1750 ppm, from about 200 to about 1500 ppm, from about 200 to about 1250 ppm, from about 200 to about 1000 ppm, from about 200 to about 900 ppm, from about 200 to about 800 ppm, from about 200 to about 700 ppm, from about 200 to about 600 ppm, from about 200 to about 500 ppm, from about 200 to about 400 ppm, or from about 200 to about 300 ppm, of the overall aqueous content of the fluid reservoir.

The at least one chlorine-containing species may be present in an amount of from about 300 to about 4500 ppm, from about 300 to about 4250 ppm, from about 300 to about 4000 ppm, from about 300 to about 3750 ppm, from about 300 to about 3500 ppm, from about 300 to about 3250 ppm, from about 300 to about 3000 ppm, from about 300 to about 2750 ppm, from about 300 to about 2500 ppm, from about 300 to about 2250 ppm, from about 300 to about 2000 ppm, from about 300 to about 1750 ppm, from about 300 to about 1500 ppm, from about 300 to about 1250 ppm, from about 300 to about 1000 ppm, from about 300 to about 900 ppm, from about 300 to about 800 ppm, from about 300 to about 700 ppm, from about 300 to about 600 ppm, from about 300 to about 500 ppm, or from about 300 to about 400 ppm, of the overall aqueous content of the fluid reservoir.

The at least one chlorine-containing species may be present in an amount of from about 400 to about 4500 ppm, from about 400 to about 4250 ppm, from about 400 to about 4000 ppm, from about 400 to about 3750 ppm, from about 400 to about 3500 ppm, from about 400 to about 3250 ppm, from about 400 to about 3000 ppm, from about 400 to about 2750 ppm, from about 400 to about 2500 ppm, from about 400 to about 2250 ppm, from about 400 to about 2000 ppm, from about 400 to about 1750 ppm, from about 400 to about 1500 ppm, from about 400 to about 1250 ppm, from about 400 to about 1000 ppm, from about 400 to about 900 ppm, from about 400 to about 800 ppm, from about 400 to about 700 ppm, from about 400 to about 600 ppm, or from about 400 to about 500 ppm, of the overall aqueous content of the fluid reservoir.

The at least one chlorine-containing species may be present in an amount of from about 500 to about 4500 ppm, from about 500 to about 4250 ppm, from about 500 to about 4000 ppm, from about 500 to about 3750 ppm, from about 500 to about 3500 ppm, from about 500 to about 3250 ppm, from about 500 to about 3000 ppm, from about 500 to about 2750 ppm, from about 500 to about 2500 ppm, from about 500 to about 2250 ppm, from about 500 to about 2000 ppm, from about 500 to about 1750 ppm, from about 500 to about 1500 ppm, from about 500 to about 1250 ppm, from about 500 to about 1000 ppm, from about 500 to about 900 ppm, from about 500 to about 800 ppm, from about 500 to about 700 ppm, or from about 500 to about 600 ppm, of the overall aqueous content of the fluid reservoir.

The at least one chlorine-containing species may be present in an amount of from about 600 to about 4500 ppm, from about 600 to about 4250 ppm, from about 600 to about 4000 ppm, from about 600 to about 3750 ppm, from about 600 to about 3500 ppm, from about 600 to about 3250 ppm, from about 600 to about 3000 ppm, from about 600 to about 2750 ppm, from about 600 to about 2500 ppm, from about 600 to about 2250 ppm, from about 600 to about 2000 ppm, from about 600 to about 1750 ppm, from about 600 to about 1500 ppm, from about 600 to about 1250 ppm, from about 600 to about 1000 ppm, from about 600 to about 900 ppm, from about 600 to about 800 ppm, or from about 600 to about 700 ppm, of the overall aqueous content of the fluid reservoir.

The at least one chlorine-containing species may be present in an amount of from about 700 to about 4500 ppm, from about 700 to about 4250 ppm, from about 700 to about 4000 ppm, from about 700 to about 3750 ppm, from about 700 to about 3500 ppm, from about 700 to about 3250 ppm, from about 700 to about 3000 ppm, from about 700 to about 2750 ppm, from about 700 to about 2500 ppm, from about 700 to about 2250 ppm, from about 700 to about 2000 ppm, from about 700 to about 1750 ppm, from about 700 to about 1500 ppm, from about 700 to about 1250 ppm, from about 700 to about 1000 ppm, from about 700 to about 900 ppm, or from about 700 to about 800 ppm, of the overall aqueous content of the fluid reservoir.

The at least one chlorine-containing species may be present in an amount of from about 800 to about 4500 ppm, from about 800 to about 4250 ppm, from about 800 to about 4000 ppm, from about 800 to about 3750 ppm, from about 800 to about 3500 ppm, from about 800 to about 3250 ppm, from about 800 to about 3000 ppm, from about 800 to about 2750 ppm, from about 800 to about 2500 ppm, from about 800 to about 2250 ppm, from about 800 to about 2000 ppm, from about 800 to about 1750 ppm, from about 800 to about 1500 ppm, from about 800 to about 1250 ppm, from about 800 to about 1000 ppm, or from about 800 to about 900 ppm, of the overall aqueous content of the fluid reservoir.

The at least one chlorine-containing species may be present in an amount of from about 900 to about 4500 ppm, from about 900 to about 4250 ppm, from about 900 to about 4000 ppm, from about 900 to about 3750 ppm, from about 900 to about 3500 ppm, from about 900 to about 3250 ppm, from about 900 to about 3000 ppm, from about 900 to about 2750 ppm, from about 900 to about 2500 ppm, from about 900 to about 2250 ppm, from about 900 to about 2000 ppm, from about 900 to about 1750 ppm, from about 900 to about 1500 ppm, from about 900 to about 1250 ppm, or from about 900 to about 1000 ppm, of the overall aqueous content of the fluid reservoir.

The at least one chlorine-containing species may be present in an amount of from about 1000 to about 4500 ppm, from about 1000 to about 4250 ppm, from about 1000 to about 4000 ppm, from about 1000 to about 3750 ppm, from about 1000 to about 3500 ppm, from about 1000 to about 3250 ppm, from about 1000 to about 3000 ppm, from about 1000 to about 2750 ppm, from about 1000 to about 2500 ppm, from about 1000 to about 2250 ppm, from about 1000 to about 2000 ppm, from about 1000 to about 1750 ppm, from about 1000 to about 1500 ppm, or from about 1000 to about 1250 ppm, of the overall aqueous content of the fluid reservoir.

The at least one chlorine-containing species may be present in an amount of from about 1250 to about 4500 ppm, from about 1250 to about 4250 ppm, from about 1250 to about 4000 ppm, from about 1250 to about 3750 ppm, from about 1250 to about 3500 ppm, from about 1250 to about 3250 ppm, from about 1250 to about 3000 ppm, from about 1250 to about 2750 ppm, from about 1250 to about 2500 ppm, from about 1250 to about 2250 ppm, from about 1250 to about 2000 ppm, from about 1250 to about 1750 ppm, or from about 1250 to about 1500 ppm, of the overall aqueous content of the fluid reservoir.

The at least one chlorine-containing species may be present in an amount of from about 1500 to about 4500 ppm, from about 1500 to about 4250 ppm, from about 1500 to about 4000 ppm, from about 1500 to about 3750 ppm, from about 1500 to about 3500 ppm, from about 1500 to about 3250 ppm, from about 1500 to about 3000 ppm, from about 1500 to about 2750 ppm, from about 1500 to about 2500 ppm, from about 1500 to about 2250 ppm, from about 1500 to about 2000 ppm, or from about 1500 to about 1750 ppm, of the overall aqueous content of the fluid reservoir.

The at least one chlorine-containing species may be present in an amount of from about 1750 to about 4500 ppm, from about 1750 to about 4250 ppm, from about 1750 to about 4000 ppm, from about 1750 to about 3750 ppm, from about 1750 to about 3500 ppm, from about 1750 to about 3250 ppm, from about 1750 to about 3000 ppm, from about 1750 to about 2750 ppm, from about 1750 to about 2500 ppm, from about 1750 to about 2250 ppm, or from about 1750 to about 2000 ppm, of the overall aqueous content of the fluid reservoir.

The at least one chlorine-containing species may be present in an amount of from about 2000 to about 4500 ppm, from about 2000 to about 4250 ppm, from about 2000 to about 4000 ppm, from about 2000 to about 3750 ppm, from about 2000 to about 3500 ppm, from about 2000 to about 3250 ppm, from about 2000 to about 3000 ppm, from about 2000 to about 2750 ppm, from about 2000 to about 2500 ppm, or from about 2000 to about 2250 ppm, of the overall aqueous content of the fluid reservoir.

The at least one chlorine-containing species may be present in an amount of from about 2250 to about 4500 ppm, from about 2250 to about 4250 ppm, from about 2250 to about 4000 ppm, from about 2250 to about 3750 ppm, from about 2250 to about 3500 ppm, from about 2250 to about 3250 ppm, from about 2250 to about 3000 ppm, from about 2250 to about 2750 ppm, or from about 2250 to about 2500 ppm, of the overall aqueous content of the fluid reservoir.

The at least one chlorine-containing species may be present in an amount of from about 2500 to about 4500 ppm, from about 2500 to about 4250 ppm, from about 2500 to about 4000 ppm, from about 2500 to about 3750 ppm, from about 2500 to about 3500 ppm, from about 2500 to about 3250 ppm, from about 2500 to about 3000 ppm, or from about 2500 to about 2750 ppm, of the overall aqueous content of the fluid reservoir.

The at least one chlorine-containing species may be present in an amount of from about 2750 to about 4500 ppm, from about 2750 to about 4250 ppm, from about 2750 to about 4000 ppm, from about 2750 to about 3750 ppm, from about 2750 to about 3500 ppm, from about 2750 to about 3250 ppm, or from about 2750 to about 3000 ppm, of the overall aqueous content of the fluid reservoir.

The at least one chlorine-containing species may be present in an amount of from about 3000 to about 4500 ppm, from about 3000 to about 4250 ppm, from about 3000 to about 4000 ppm, from about 3000 to about 3750 ppm, from about 3000 to about 3500 ppm, or from about 3000 to about 3250 ppm, of the overall aqueous content of the fluid reservoir.

The at least one chlorine-containing species may be present in an amount of from about 3250 to about 4500 ppm, from about 3250 to about 4250 ppm, from about 3250 to about 4000 ppm, from about 3250 to about 3750 ppm, or from about 3250 to about 3500 ppm, of the overall aqueous content of the fluid reservoir.

The at least one chlorine-containing species may be present in an amount of from about 3500 to about 4500 ppm, from about 3500 to about 4250 ppm, from about 3500 to about 4000 ppm, or from about 3500 to about 3750 ppm, of the overall aqueous content of the fluid reservoir.

The at least one chlorine-containing species may be present in an amount of from about 3750 to about 4500 ppm, from about 3750 to about 4250 ppm, or from about 3750 to about 4000 ppm, of the overall aqueous content of the fluid reservoir.

The at least one chlorine-containing species may be present in an amount of from about 4000 to about 4500 ppm, or from about 4000 to about 4250 ppm, of the overall aqueous content of the fluid reservoir.

The at least one chlorine-containing species may be present in an amount of from about 4250 to about 4500 ppm, of the overall aqueous content of the fluid reservoir.

In particular, the at least one chlorine-containing species may be present in an amount of no more than about 4500 ppm of the overall aqueous content of the fluid reservoir. For example, the at least one chlorine-containing species may be present in an amount of between about 1800 ppm and about 2800 ppm, about 2000 ppm and about 2500 ppm, about 2100 ppm and about 2400 ppm, or between about 2200 ppm and about 2300 ppm, of the overall aqueous content of the fluid reservoir. For example, the at least one chlorine-containing species may be present in an amount of about 1800 ppm, about 1900 ppm, about 2000 ppm, about 2100 ppm, 2200 ppm, about 2300 ppm, about 2400 ppm, about 2500 ppm, about 2600 ppm, about 2700 ppm, or about 2800 ppm, of the overall aqueous content of the fluid reservoir. For example, the at least one chlorine-containing species may be present in an amount of about 1800 ppm, about 1900 ppm, about 2000 ppm, about 2100 ppm, 2200 ppm, about 2300 ppm, about 2400 ppm, about 2500 ppm, about 2600 ppm, about 2700 ppm, or at least about 2800 ppm,
of the overall aqueous content of the fluid reservoir. For example, the at least one chlorine-containing species may be present in an amount of about 1800 ppm, about 1900 ppm, about 2000 ppm, about 2100 ppm, 2200 ppm, about 2300 ppm, about 2400 ppm, about 2500 ppm, about 2600 ppm, about 2700 ppm, or no more than about 2800 ppm, of the overall aqueous content of the fluid reservoir.

In some embodiments, there is provided a packaged intermittent urinary catheter comprising: a single use catheter; a packaging surrounding the catheter; and a fluid reservoir holding a lubricant which, upon rupturing the fluid reservoir, is transferred into the packaging to immerse at least a portion of the catheter in the lubricant to prepare the catheter for use, wherein the lubricant comprises an antimicrobial medium, further wherein the antimicrobial medium comprises an aqueous chlorine solution comprising at least one chlorine-containing species independently chosen from: hypochlorous acid, at least one hypochlorite salt, chlorine dioxide, at least one chlorhexidine salt, and combinations thereof, and the at least one chlorine-containing species is present in an amount of between 100 and 4500 ppm of the overall aqueous content of the fluid reservoir.

In embodiments comprising hypochlorous acid, the hypochlorous acid may be present in an amount of between about 100 and about 4500 ppm, about 150 and about 4400 ppm, about 200 and about 4300 ppm, about 250 and about 4200 ppm, about 300 and about 4100 ppm, about 350 and about 4000 ppm, about 400 and about 3900 ppm, about 450 and about 3800 ppm, about 500 and about 3700 ppm, about 550 and about 3600 ppm, about 600 and about 3500 ppm, about 650 and about 3400 ppm, about 700 and about 3300 ppm, about 750 and about 3200 ppm, about 800 and about 3100 ppm, about 850 and about 3000 ppm, about 900 and about 2900 ppm, about 950 and about 2800 ppm, about 1000 and about 2700 ppm, about 1050 and about 2600 ppm, about 1100 and about 2500 ppm, about 1150 and about 2400 ppm, about 1200 and about 2300 ppm, about 1250 and about 2200 ppm, about 1300 and about 2100 ppm, about 1350 and about 2000 ppm, about 1400 and about 1900 ppm, about 1450 and about 1800 ppm, about 1500 and about 1700 ppm, or about 1600 ppm, of the overall aqueous content of the fluid reservoir.

In embodiments comprising hypochlorous acid, the hypochlorous acid may be present in an amount of about 100 ppm, about 150 ppm, about 200 ppm, about 250 ppm, about 300 ppm, about 350 ppm, about 400 ppm, about 450 ppm, about 500 ppm, about 550 ppm, about 600 ppm, about 650 ppm, about 700 ppm, about 750 ppm, about 800 ppm, about 850 ppm, about 900 ppm, about 950 ppm, about 1000 ppm, about 1050 ppm, about 1100 ppm, about 1150 ppm, about 1200 ppm, about 1250 ppm, about 1300 ppm, about 1350 ppm, about 1400 ppm, about 1450 ppm, about 1500 ppm, about 1550 ppm, about 1600 ppm, about 1650 ppm, about 1700 ppm, about 1750 ppm, about 1800 ppm, about 1850 ppm, about 1900 ppm, about 1950 ppm, about 2000 ppm, about 2050 ppm, about 2100 ppm, about 2150 ppm, about 2200 ppm, about 2250 ppm, about 2300 ppm, about 2350 ppm, about 2400 ppm, about 2450 ppm, about 2500 ppm, about 2550 ppm, about 2600 ppm, about 2650 ppm, about 2700 ppm, about 2750 ppm, about 2800 ppm, about 2850 ppm, about 2900 ppm, about 2950 ppm, about 3000 ppm, about 3050 ppm, about 3100 ppm, about 3150 ppm, about 3200 ppm, about 3250 ppm, about 3300 ppm, about 3350 ppm, about 3400 ppm, about 3450 ppm, about 3500 ppm, about 3550 ppm, about 3600 ppm, about 3650 ppm, about 3700 ppm, about 3750 ppm, about 3800 ppm, about 3850 ppm, about 3900 ppm, about 3950 ppm, about 4000 ppm, about 4050 ppm, about 4100 ppm, about 4150 ppm, about 4200 ppm, about 4250 ppm, about 4300 ppm, about 4350 ppm, about 4400 ppm, about 4450 ppm, or about 4500 ppm, of the overall aqueous content of the fluid reservoir.

In embodiments comprising hypochlorous acid, the hypochlorous acid may be present in an amount of at least about 100 ppm, about 150 ppm, about 200 ppm, about 250 ppm, about 300 ppm, about 350 ppm, about 400 ppm, about 450 ppm, about 500 ppm, about 550 ppm, about 600 ppm, about 650 ppm, about 700 ppm, about 750 ppm, about 800 ppm, about 850 ppm, about 900 ppm, about 950 ppm, about 1000 ppm, about 1050 ppm, about 1100 ppm, about 1150 ppm, about 1200 ppm, about 1250 ppm, about 1300 ppm, about 1350 ppm, about 1400 ppm, about 1450 ppm, about 1500 ppm, about 1550 ppm, about 1600 ppm, about 1650 ppm, about 1700 ppm, about 1750 ppm, about 1800 ppm, about 1850 ppm, about 1900 ppm, about 1950 ppm, about 2000 ppm, about 2050 ppm, about 2100 ppm, about 2150 ppm, about 2200 ppm, about 2250 ppm, about 2300 ppm, about 2350 ppm, about 2400 ppm, about 2450 ppm, about 2500 ppm, about 2550 ppm, about 2600 ppm, about 2650 ppm, about 2700 ppm, about 2750 ppm, about 2800 ppm, about 2850 ppm, about 2900 ppm, about 2950 ppm, about 3000 ppm, about 3050 ppm, about 3100 ppm, about 3150 ppm, about 3200 ppm, about 3250 ppm, about 3300 ppm, about 3350 ppm, about 3400 ppm, about 3450 ppm, about 3500 ppm, about 3550 ppm, about 3600 ppm, about 3650 ppm, about 3700 ppm, about 3750 ppm, about 3800 ppm, about 3850 ppm, about 3900 ppm, about 3950 ppm, about 4000 ppm, about 4050 ppm, about 4100 ppm, about 4150 ppm, about 4200 ppm, about 4250 ppm, about 4300 ppm, about 4350 ppm, about 4400 ppm, about 4450 ppm, or at least about 4500 ppm, of the overall aqueous content of the fluid reservoir.

In embodiments comprising hypochlorous acid, the hypochlorous acid may be present in an amount of no more than about 100 ppm, about 150 ppm, about 200 ppm, about 250 ppm, about 300 ppm, about 350 ppm, about 400 ppm, about 450 ppm, about 500 ppm, about 550 ppm, about 600 ppm, about 650 ppm, about 700 ppm, about 750 ppm, about 800 ppm, about 850 ppm, about 900 ppm, about 950 ppm, about 1000 ppm, about 1050 ppm, about 1100 ppm, about 1150 ppm, about 1200 ppm, about 1250 ppm, about 1300 ppm, about 1350 ppm, about 1400 ppm, about 1450 ppm, about 1500 ppm, about 1550 ppm, about 1600 ppm, about 1650 ppm, about 1700 ppm, about 1750 ppm, about 1800 ppm, about 1850 ppm, about 1900 ppm, about 1950 ppm, about 2000 ppm, about 2050 ppm, about 2100 ppm, about 2150 ppm. about 2200 ppm, about 2250 ppm, about 2300 ppm, about 2350 ppm, about 2400 ppm, about 2450 ppm, about 2500 ppm, about 2550 ppm, about 2600 ppm, about 2650 ppm, about 2700 ppm, about 2750 ppm, about 2800 ppm, about 2850 ppm, about 2900 ppm, about 2950 ppm, about 3000 ppm, about 3050 ppm, about 3100 ppm, about 3150 ppm, about 3200 ppm, about 3250 ppm, about 3300 ppm, about 3350 ppm, about 3400 ppm, about 3450 ppm, about 3500 ppm, about 3550 ppm, about 3600 ppm, about 3650 ppm, about 3700 ppm, about 3750 ppm, about 3800 ppm, about 3850 ppm, about 3900 ppm, about 3950 ppm, about 4000 ppm, about 4050 ppm, about 4100 ppm, about 4150 ppm, about 4200 ppm, about 4250 ppm, about 4300 ppm, about 4350 ppm, about 4400 ppm, about 4450 ppm, or no more than about 4500 ppm, of the overall aqueous content of the fluid reservoir.

In embodiments comprising hypochlorous acid, the hypochlorous acid may be present in an amount of from about 50 to about 4500 ppm, from about 50 to about 4250 ppm, from about 50 to about 4000 ppm, from about 50 to about 3750 ppm, from about 50 to about 3500 ppm, from about 50 to about 3250 ppm, from about 50 to about 3000 ppm, from about 50 to about 2750 ppm, from about 50 to about 2500 ppm, from about 50 to about 2250 ppm, from about 50 to about 2000 ppm, from about 50 to about 1750 ppm, from about 50 to about 1500 ppm, from about 50 to about 1250 ppm, from about 50 to about 1000 ppm, from about 50 to about 900 ppm, from about 50 to about 800 ppm, from about 50 to about 700 ppm, from about 50 to about 600 ppm, from about 50 to about 500 ppm, from about 50 to about 400 ppm, from about 50 to about 300 ppm, from about 50 to about 200 ppm, or from about 50 to about 100 ppm, of the overall aqueous content of the fluid reservoir.

In embodiments comprising hypochlorous acid, the hypochlorous acid may be present in an amount of from about 100 to about 4500 ppm, from about 100 to about 4250 ppm, from about 100 to about 4000 ppm, from about 100 to about 3750 ppm, from about 100 to about 3500 ppm, from about 100 to about 3250 ppm, from about 100 to about 3000 ppm, from about 100 to about 2750 ppm, from about 100 to about 2500 ppm, from about 100 to about 2250 ppm, from about 100 to about 2000 ppm, from about 100 to about 1750 ppm, from about 100 to about 1500 ppm, from about 100 to about 1250 ppm, from about 100 to about 1000 ppm, from about 100 to about 900 ppm, from about 100 to about 800 ppm, from about 100 to about 700 ppm, from about 100 to about 600 ppm, from about 100 to about 500 ppm, from about 100 to about 400 ppm, from about 100 to about 300 ppm, or from about 100 to about 200 ppm, of the overall aqueous content of the fluid reservoir.

In embodiments comprising hypochlorous acid, the hypochlorous acid may be present in an amount of from about 200 to about 4500 ppm, from about 200 to about 4250 ppm, from about 200 to about 4000 ppm, from about 200 to about 3750 ppm, from about 200 to about 3500 ppm, from about 200 to about 3250 ppm, from about 200 to about 3000 ppm, from about 200 to about 2750 ppm, from about 200 to about 2500 ppm, from about 200 to about 2250 ppm, from about 200 to about 2000 ppm, from about 200 to about 1750 ppm, from about 200 to about 1500 ppm, from about 200 to about 1250 ppm, from about 200 to about 1000 ppm, from about 200 to about 900 ppm, from about 200 to about 800 ppm, from about 200 to about 700 ppm, from about 200 to about 600 ppm, from about 200 to about 500 ppm, from about 200 to about 400 ppm, or from about 200 to about 300 ppm, of the overall aqueous content of the fluid reservoir.

In embodiments comprising hypochlorous acid, the hypochlorous acid may be present in an amount of from about 300 to about 4500 ppm, from about 300 to about 4250 ppm, from about 300 to about 4000 ppm, from about 300 to about 3750 ppm, from about 300 to about 3500 ppm, from about 300 to about 3250 ppm, from about 300 to about 3000 ppm, from about 300 to about 2750 ppm, from about 300 to about 2500 ppm, from about 300 to about 2250 ppm, from about 300 to about 2000 ppm, from about 300 to about 1750 ppm, from about 300 to about 1500 ppm, from about 300 to about 1250 ppm, from about 300 to about 1000 ppm, from about 300 to about 900 ppm, from about 300 to about 800 ppm, from about 300 to about 700 ppm, from about 300 to about 600 ppm, from about 300 to about 500 ppm, or from about 300 to about 400 ppm, of the overall aqueous content of the fluid reservoir.

In embodiments comprising hypochlorous acid, the hypochlorous acid may be present in an amount of from about 400 to about 4500 ppm, from about 400 to about 4250 ppm, from about 400 to about 4000 ppm, from about 400 to about 3750 ppm, from about 400 to about 3500 ppm, from about 400 to about 3250 ppm, from about 400 to about 3000 ppm, from about 400 to about 2750 ppm, from about 400 to about 2500 ppm, from about 400 to about 2250 ppm, from about 400 to about 2000 ppm, from about 400 to about 1750 ppm, from about 400 to about 1500 ppm, from about 400 to about 1250 ppm, from about 400 to about 1000 ppm, from about 400 to about 900 ppm, from about 400 to about 800 ppm, from about 400 to about 700 ppm, from about 400 to about 600 ppm, or from about 400 to about 500 ppm, of the overall aqueous content of the fluid reservoir.

In embodiments comprising hypochlorous acid, the hypochlorous acid may be present in an amount of from about 500 to about 4500 ppm, from about 500 to about 4250 ppm, from about 500 to about 4000 ppm, from about 500 to about 3750 ppm, from about 500 to about 3500 ppm, from about 500 to about 3250 ppm, from about 500 to about 3000 ppm, from about 500 to about 2750 ppm, from about 500 to about 2500 ppm, from about 500 to about 2250 ppm, from about 500 to about 2000 ppm, from about 500 to about 1750 ppm, from about 500 to about 1500 ppm, from about 500 to about 1250 ppm, from about 500 to about 1000 ppm, from about 500 to about 900 ppm, from about 500 to about 800 ppm, from about 500 to about 700 ppm, or from about 500 to about 600 ppm, of the overall aqueous content of the fluid reservoir.

In embodiments comprising hypochlorous acid, the hypochlorous acid may be present in an amount of from about 600 to about 4500 ppm, from about 600 to about 4250 ppm, from about 600 to about 4000 ppm, from about 600 to about 3750 ppm, from about 600 to about 3500 ppm, from about 600 to about 3250 ppm, from about 600 to about 3000 ppm, from about 600 to about 2750 ppm, from about 600 to about 2500 ppm, from about 600 to about 2250 ppm, from about 600 to about 2000 ppm, from about 600 to about 1750 ppm, from about 600 to about 1500 ppm, from about 600 to about 1250 ppm, from about 600 to about 1000 ppm, from about 600 to about 900 ppm, from about 600 to about 800 ppm, or from about 600 to about 700 ppm, of the overall aqueous content of the fluid reservoir.

In embodiments comprising hypochlorous acid, the hypochlorous acid may be present in an amount of from about 700 to about 4500 ppm, from about 700 to about 4250 ppm, from about 700 to about 4000 ppm, from about 700 to about 3750 ppm, from about 700 to about 3500 ppm, from about 700 to about 3250 ppm, from about 700 to about 3000 ppm, from about 700 to about 2750 ppm, from about 700 to about 2500 ppm, from about 700 to about 2250 ppm, from about 700 to about 2000 ppm, from about 700 to about 1750 ppm, from about 700 to about 1500 ppm, from about 700 to about 1250 ppm, from about 700 to about 1000 ppm, from about 700 to about 900 ppm, or from about 700 to about 800 ppm, of the overall aqueous content of the fluid reservoir.

In embodiments comprising hypochlorous acid, the hypochlorous acid may be present in an amount of from about 800 to about 4500 ppm, from about 800 to about 4250 ppm, from about 800 to about 4000 ppm, from about 800 to about 3750 ppm, from about 800 to about 3500 ppm, from about 800 to about 3250 ppm, from about 800 to about 3000 ppm, from about 800 to about 2750 ppm, from about 800 to about 2500 ppm, from about 800 to about 2250 ppm, from about 800 to about 2000 ppm, from about 800 to about 1750 ppm, from about 800 to about 1500 ppm, from about 800 to about 1250 ppm, from about 800 to about 1000 ppm, or from about 800 to about 900 ppm, of the overall aqueous content of the fluid reservoir.

In embodiments comprising hypochlorous acid, the hypochlorous acid may be present in an amount of from about 900 to about 4500 ppm, from about 900 to about 4250 ppm, from about 900 to about 4000 ppm, from about 900 to about 3750 ppm, from about 900 to about 3500 ppm, from about 900 to about 3250 ppm, from about 900 to about 3000 ppm, from about 900 to about 2750 ppm, from about 900 to about 2500 ppm, from about 900 to about 2250 ppm, from about 900 to about 2000 ppm, from about 900 to about 1750 ppm, from about 900 to about 1500 ppm, from about 900 to about 1250 ppm, or from about 900 to about 1000 ppm, of the overall aqueous content of the fluid reservoir.

In embodiments comprising hypochlorous acid, the hypochlorous acid may be present in an amount of from about 1000 to about 4500 ppm, from about 1000 to about 4250 ppm, from about 1000 to about 4000 ppm, from about 1000 to about 3750 ppm, from about 1000 to about 3500 ppm, from about 1000 to about 3250 ppm, from about 1000 to about 3000 ppm, from about 1000 to about 2750 ppm, from about 1000 to about 2500 ppm, from about 1000 to about 2250 ppm, from about 1000 to about 2000 ppm, from about 1000 to about 1750 ppm. from about 1000 to about 1500 ppm, or from about 1000 to about 1250 ppm, of the overall aqueous content of the fluid reservoir.

In embodiments comprising hypochlorous acid, the hypochlorous acid may be present in an amount of from about 1250 to about 4500 ppm, from about 1250 to about 4250 ppm, from about 1250 to about 4000 ppm, from about 1250 to about 3750 ppm, from about 1250 to about 3500 ppm, from about 1250 to about 3250 ppm, from about 1250 to about 3000 ppm, from about 1250 to about 2750 ppm, from about 1250 to about 2500 ppm, from about 1250 to about 2250 ppm, from about 1250 to about 2000 ppm, from about 1250 to about 1750 ppm, or from about 1250 to about 1500 ppm, of the overall aqueous content of the fluid reservoir.

In embodiments comprising hypochlorous acid, the hypochlorous acid may be present in an amount of from about 1500 to about 4500 ppm, from about 1500 to about 4250 ppm, from about 1500 to about 4000 ppm, from about 1500 to about 3750 ppm, from about 1500 to about 3500 ppm, from about 1500 to about 3250 ppm, from about 1500 to about 3000 ppm, from about 1500 to about 2750 ppm, from about 1500 to about 2500 ppm, from about 1500 to about 2250 ppm, from about 1500 to about 2000 ppm, or from about 1500 to about 1750 ppm, of the overall aqueous content of the fluid reservoir.

In embodiments comprising hypochlorous acid, the hypochlorous acid may be present in an amount of from about 1750 to about 4500 ppm, from about 1750 to about 4250 ppm, from about 1750 to about 4000 ppm, from about 1750 to about 3750 ppm, from about 1750 to about 3500 ppm, from about 1750 to about 3250 ppm, from about 1750 to about 3000 ppm, from about 1750 to about 2750 ppm, from about 1750 to about 2500 ppm, from about 1750 to about 2250 ppm, or from about 1750 to about 2000 ppm, of the overall aqueous content of the fluid reservoir.

In embodiments comprising hypochlorous acid, the hypochlorous acid may be present in an amount of from about 2000 to about 4500 ppm, from about 2000 to about 4250 ppm, from about 2000 to about 4000 ppm, from about 2000 to about 3750 ppm, from about 2000 to about 3500 ppm, from about 2000 to about 3250 ppm, from about 2000 to about 3000 ppm, from about 2000 to about 2750 ppm, from about 2000 to about 2500 ppm, or from about 2000 to about 2250 ppm, of the overall aqueous content of the fluid reservoir.

In embodiments comprising hypochlorous acid, the hypochlorous acid may be present in an amount of from about 2250 to about 4500 ppm, from about 2250 to about 4250 ppm, from about 2250 to about 4000 ppm, from about 2250 to about 3750 ppm, from about 2250 to about 3500 ppm, from about 2250 to about 3250 ppm, from about 2250 to about 3000 ppm, from about 2250 to about 2750 ppm, or from about 2250 to about 2500 ppm, of the overall aqueous content of the fluid reservoir.

In embodiments comprising hypochlorous acid, the hypochlorous acid may be present in an amount of from about 2500 to about 4500 ppm, from about 2500 to about 4250 ppm, from about 2500 to about 4000 ppm, from about 2500 to about 3750 ppm, from about 2500 to about 3500 ppm, from about 2500 to about 3250 ppm, from about 2500 to about 3000 ppm, or from about 2500 to about 2750 ppm, of the overall aqueous content of the fluid reservoir.

In embodiments comprising hypochlorous acid, the hypochlorous acid may be present in an amount of from about 2750 to about 4500 ppm, from about 2750 to about 4250 ppm, from about 2750 to about 4000 ppm, from about 2750 to about 3750 ppm, from about 2750 to about 3500 ppm, from about 2750 to about 3250 ppm, or from about 2750 to about 3000 ppm, of the overall aqueous content of the fluid reservoir.

In embodiments comprising hypochlorous acid, the hypochlorous acid may be present in an amount of from about 3000 to about 4500 ppm, from about 3000 to about 4250 ppm, from about 3000 to about 4000 ppm, from about 3000 to about 3750 ppm, from about 3000 to about 3500 ppm, or from about 3000 to about 3250 ppm, of the overall aqueous content of the fluid reservoir.

In embodiments comprising hypochlorous acid, the hypochlorous acid may be present in an amount of from about 3250 to about 4500 ppm, from about 3250 to about 4250 ppm, from about 3250 to about 4000 ppm, from about 3250 to about 3750 ppm, or from about 3250 to about 3500 ppm, of the overall aqueous content of the fluid reservoir.

In embodiments comprising hypochlorous acid, the hypochlorous acid may be present in an amount of from about 3500 to about 4500 ppm, from about 3500 to about 4250 ppm, from about 3500 to about 4000 ppm, or from about 3500 to about 3750 ppm, of the overall aqueous content of the fluid reservoir.

In embodiments comprising hypochlorous acid, the hypochlorous acid may be present in an amount of from about 3750 to about 4500 ppm, from about 3750 to about 4250 ppm, or from about 3750 to about 4000 ppm, of the overall aqueous content of the fluid reservoir.

In embodiments comprising hypochlorous acid, the hypochlorous acid may be present in an amount of from about 4000 to about 4500 ppm, or from about 4000 to about 4250 ppm, of the overall aqueous content of the fluid reservoir.

In embodiments comprising hypochlorous acid, the hypochlorous acid may be present in an amount of from about 4250 to about 4500 ppm, of the overall aqueous content of the fluid reservoir.

Advantageously, the concentrations of the at least one chlorine-containing species are relatively low compared to sterilisation mediums contained within conventional packaged reusable intermittent catheters. This is because the sterilisation medium of a conventional packaged reusable catheter concerns sterilisation of a catheter after a first use and after each repeated use to provide a catheter sterilised ready for re-use. Thus, conventional reusable catheters require a higher concentration of antimicrobial species to ensure sterilisation after multiple uses of the catheter.

In some embodiments, there is provided a packaged intermittent urinary catheter comprising: a single use catheter; a packaging surrounding the catheter; and a fluid reservoir holding a lubricant which, upon rupturing the fluid reservoir, is transferred into the packaging to immerse at least a portion of the catheter in the lubricant to prepare the catheter for use, wherein the lubricant comprises an antimicrobial medium, further wherein the antimicrobial medium comprises an aqueous chlorine solution comprising at least one chlorine-containing species, wherein the at least one chlorine-containing species is hypochlorous acid, and the hypochlorous acid is present in an amount of between 100 and 4500 ppm of the overall aqueous content of the fluid reservoir.

The present invention concerns a packaged single use catheter comprising a lubricant which also has an antimicrobial function. As such, the lubricant of the present invention is not required to provide a sterilisation effect upon all of, or at least a significant portion of, the catheter following multiple uses, as is the case with sterilisation mediums contained within packaged reusable intermittent catheters. Instead, the present invention is required to provide an antimicrobial effect when the single use catheter is in use. As such, the concentration of the at least one chlorine-containing species may be lower than that of the antimicrobial source comprised in a conventional packaged reusable catheter. For example, the at least one chlorine-containing species of the present invention may be present in an amount of no more than about 4500 ppm of the overall aqueous content of the fluid reservoir.

Further advantageously, the concentrations of the at least one chlorine-containing species of the invention provide for fast acting elimination of bacteria. This is particularly beneficial because of the short indwell period of the catheter.

The total concentration of the at least one chlorine-containing species may be from about 0.05 to about 0.50% wt./vol, about 0.10 to about 0.45% wt./vol, about 0.15 to about 0.40% wt./vol, about 0.18 to about 0.35% wt./vol, about 0.20 to about 0.30% wt./vol, about 0.22 to about 0.28% wt./vol, about 0.24 to about 0.26% wt./vol, or about 0.25% wt./vol, of the overall aqueous content of the fluid reservoir.

The total concentration of the at least one chlorine-containing species may be about 0.05% wt./vol, about 0.10% wt./vol, about 0.15% wt./vol, about 0.20% wt./vol, about 0.25% wt./vol, about 0.30% wt./vol, about 0.35% wt./vol, about 0.40% wt./vol, about 0.45% wt./vol, or about 0.50% wt./vol, of the overall aqueous content of the fluid reservoir.

The total concentration of the at least one chlorine-containing species may be at least about 0.05% wt./vol, about 0.10% wt./vol, about 0.15% wt./vol, about 0.20% wt./vol, about 0.25% wt./vol, about 0.30% wt./vol, about 0.35% wt./vol, about 0.40% wt./vol, about 0.45% wt./vol, or at least about 0.50% wt./vol, of the overall aqueous content of the fluid reservoir.

The total concentration of the at least one chlorine-containing species may be no more than about 0.05% wt./vol, about 0.10% wt./vol, about 0.15% wt./vol, about 0.20% wt./vol, about 0.25% wt./vol, about 0.30% wt./vol, about 0.35% wt./vol, about 0.40% wt./vol, about 0.45% wt./vol, or no more than about 0.50% wt./vol, of the overall aqueous content of the fluid reservoir.

The total concentration of the at least one chlorine-containing species may be from about 0.05% wt./vol to about 0.50% wt./vol, from about 0.05% wt./vol to about 0.45% wt./vol, from about 0.05% wt./vol to about 0.40% wt./vol, from about 0.05% wt./vol to about 0.35% wt./vol, from about 0.05% wt./vol to about 0.30% wt./vol, from about 0.05% wt./vol to about 0.25% wt./vol, from about 0.05% wt./vol to about 0.20% wt./vol, from about 0.05% wt./vol to about 0.15% wt./vol, or from about 0.05% wt./vol to about 0.10% wt./vol, of the overall aqueous content of the fluid reservoir.

The total concentration of the at least one chlorine-containing species may be from about 0.10% wt./vol to about 0.50% wt./vol, from about 0.10% wt./vol to about 0.45% wt./vol, from about 0.10% wt./vol to about 0.40% wt./vol, from about 0.10% wt./vol to about 0.35% wt./vol, from about 0.10% wt./vol to about 0.30% wt./vol, from about 0.10% wt./vol to about 0.25% wt./vol, from about 0.10% wt./vol to about 0.20% wt./vol, or from about 0.10% wt./vol to about 0.15% wt./vol, of the overall aqueous content of the fluid reservoir.

The total concentration of the at least one chlorine-containing species may be from about 0.15% wt./vol to about 0.50% wt./vol, from about 0.15% wt./vol to about 0.45% wt./vol, from about 0.15% wt./vol to about 0.40% wt./vol, from about 0.15% wt./vol to about 0.35% wt./vol, from about 0.15% wt./vol to about 0.30% wt./vol, from about 0.15% wt./vol to about 0.25% wt./vol, or from about 0.15% wt./vol to about 0.20% wt./vol, of the overall aqueous content of the fluid reservoir.

The total concentration of the at least one chlorine-containing species may be from about 0.20% wt./vol to about 0.50% wt./vol, from about 0.20% wt./vol to about 0.45% wt./vol, from about 0.20% wt./vol to about 0.40% wt./vol, from about 0.20% wt./vol to about 0.35% wt./vol, from about 0.20% wt./vol to about 0.30% wt./vol, or from about 0.20% wt./vol to about 0.25% wt./vol, of the overall aqueous content of the fluid reservoir.

The total concentration of the at least one chlorine-containing species may be from about 0.25% wt./vol to about 0.50% wt./vol, from about 0.25% wt./vol to about 0.45% wt./vol, from about 0.25% wt./vol to about 0.40% wt./vol, from about 0.25% wt./vol to about 0.35% wt./vol, or from about 0.25% wt./vol to about 0.30% wt./vol, of the overall aqueous content of the fluid reservoir.

The total concentration of the at least one chlorine-containing species may be from about 0.30% wt./vol to about 0.50% wt./vol, from about 0.30% wt./vol to about 0.45% wt./vol, from about 0.30% wt./vol to about 0.40% wt./vol, or from about 0.30% wt./vol to about 0.35% wt./vol, of the overall aqueous content of the fluid reservoir.

The total concentration of the at least one chlorine-containing species may be from about 0.35% wt./vol to about 0.50% wt./vol, from about 0.35% wt./vol to about 0.45% wt./vol, or from about 0.35% wt./vol to about 0.40% wt./vol, of the overall aqueous content of the fluid reservoir.

The total concentration of the at least one chlorine-containing species may be from about 0.40% wt./vol to about 0.50% wt./vol, or from about 0.40% wt./vol to about 0.45% wt./vol, of the overall aqueous content of the fluid reservoir.

The total concentration of the at least one chlorine-containing species may be from about 0.45% wt./vol to about 0.50% wt./vol, of the overall aqueous content of the fluid reservoir.

In particular, the concentration of the at least one chlorine-containing species may be no more than about 0.45% wt./vol of the overall aqueous content of the fluid reservoir. For example, the concentration of the at least one chlorine-containing species may be between about 0.18% wt./vol and about 0.28% wt./vol, about 0.20% wt./vol and about 0.25% wt./vol, about 0.21% wt./vol and about 0.24% wt./vol, or between about 0.22% wt./vol and about 0.23% wt./vol, of the overall aqueous content of the fluid reservoir. For example, the concentration of the at least one chlorine-containing species may be about 0.18% wt./vol, about 0.19% wt./vol, about 0.20% wt./vol, about 0.21% wt./vol, 0.22% wt./vol, about 0.23% wt./vol, about 0.24% wt./vol, about 0.25% wt./vol, about 0.26% wt./vol, about 0.27% wt./vol, or about 0.28 % wt./vol, of the overall aqueous content of the fluid reservoir. For example, the concentration of the at least one chlorine-containing species may be at least about 0.18% wt./vol, about 0.19% wt./vol, about 0.20% wt./vol, about 0.21% wt./vol, 0.22% wt./vol, about 0.23% wt./vol, about 0.24% wt./vol, about 0.25% wt./vol, about 0.26% wt./vol, about 0.27% wt./vol, or at least about 0.28% wt./vol, of the overall aqueous content of the fluid reservoir. For example, the concentration of the at least one chlorine-containing species may be no more than about 0.18% wt./vol, about 0.19% wt./vol, about 0.20% wt./vol, about 0.21% wt./vol, 0.22% wt./vol, about 0.23% wt./vol, about 0.24% wt./vol, about 0.25% wt./vol, about 0.26% wt./vol, about 0.27% wt./vol, or no more than about 0.28% wt./vol, of the overall aqueous content of the fluid reservoir.

In some embodiments, there is provided a packaged intermittent urinary catheter comprising: a single use catheter; a packaging surrounding the catheter; and a fluid reservoir holding a lubricant which, upon rupturing the fluid reservoir, is transferred into the packaging to immerse at least a portion of the catheter in the lubricant to prepare the catheter for use, wherein the lubricant comprises an
antimicrobial medium, further wherein the antimicrobial medium comprises an aqueous chlorine solution comprising at least one chlorine-containing species independently chosen from: hypochlorous acid, at least one hypochlorite salt, chlorine dioxide, at least one chlorhexidine salt, and combinations thereof, and the total concentration of the at least one chlorine-containing species is of from 0.05 to 0.5% wt./vol of the overall aqueous content of the fluid reservoir.

In some embodiments, there is provided a packaged intermittent urinary catheter comprising: a single use catheter; a packaging surrounding the catheter; and a fluid reservoir holding a lubricant which, upon rupturing the fluid reservoir, is transferred into the packaging to immerse at least a portion of the catheter in the lubricant to prepare the catheter for use, wherein the lubricant comprises an antimicrobial medium, further wherein the antimicrobial medium comprises an aqueous chlorine solution comprising at least one chlorine-containing species independently chosen from: hypochlorous acid, at least one hypochlorite salt, chlorine dioxide, at least one chlorhexidine salt, and combinations thereof, and the total concentration of the at least one chlorine-containing species is no more than 0.45% wt./vol of the overall aqueous content of the fluid reservoir.

In embodiments comprising hypochlorous acid, the concentration of the hypochlorous acid may be from about 0.05 to about 0.50% wt./vol, about 0.10 to about 0.45% wt./vol, about 0.15 to about 0.40% wt./vol, about 0.18 to about 0.35% wt./vol, about 0.20 to about 0.30% wt./vol, about 0.22 to about 0.28% wt./vol, about 0.24 to about 0.26% wt./vol, or about 0.25% wt./vol, of the overall aqueous content of the fluid reservoir.

In embodiments comprising hypochlorous acid, the concentration of the hypochlorous acid may be about 0.05% wt./vol, about 0.10% wt./vol, about 0.15% wt./vol, about 0.20% wt./vol, about 0.25% wt./vol, about 0.30% wt./vol, about 0.35% wt./vol, about 0.40% wt./vol, about 0.45% wt./vol, or about 0.50% wt./vol, of the overall aqueous content of the fluid reservoir.

In embodiments comprising hypochlorous acid, the concentration of the hypochlorous acid may be at least about 0.05% wt./vol, about 0.10% wt./vol, about 0.15% wt./vol, about 0.20% wt./vol, about 0.25% wt./vol, about 0.30% wt./vol, about 0.35% wt./vol, about 0.40% wt./vol, about 0.45% wt./vol, or at least about 0.50% wt./vol, of the overall aqueous content of the fluid reservoir.

In embodiments comprising hypochlorous acid, the concentration of the hypochlorous acid may be no more than about 0.05% wt./vol, about 0.10% wt./vol, about 0.15% wt./vol, about 0.20% wt./vol, about 0.25% wt./vol, about 0.30% wt./vol, about 0.35% wt./vol, about 0.40% wt./vol, about 0.45% wt./vol, or no more than about 0.50% wt./vol, of the overall aqueous content of the fluid reservoir.

In embodiments comprising hypochlorous acid, the concentration of the hypochlorous acid may be from about 0.05% wt./vol to about 0.50% wt./vol, from about 0.05% wt./vol to about 0.45% wt./vol, from about 0.05% wt./vol to about 0.40% wt./vol, from about 0.05% wt./vol to about 0.35% wt./vol, from about 0.05% wt./vol to about 0.30% wt./vol, from about 0.05% wt./vol to about 0.25% wt./vol, from about 0.05% wt./vol to about 0.20% wt./vol, from about 0.05% wt./vol to about 0.15% wt./vol, or from about 0.05% wt./vol to about 0.10% wt./vol, of the overall aqueous content of the fluid reservoir.

In embodiments comprising hypochlorous acid, the concentration of the hypochlorous acid may be from about 0.10% wt./vol to about 0.50% wt./vol, from about 0.10% wt./vol to about 0.45% wt./vol, from about 0.10% wt./vol to about 0.40% wt./vol, from about 0.10% wt./vol to about 0.35% wt./vol, from about 0.10% wt./vol to about 0.30% wt./vol, from about 0.10% wt./vol to about 0.25% wt./vol, from about 0.10% wt./vol to about 0.20% wt./vol, or from about 0.10% wt./vol to about 0.15% wt./vol, of the overall aqueous content of the fluid reservoir.

In embodiments comprising hypochlorous acid, the concentration of the hypochlorous acid may be from about 0.15% wt./vol to about 0.50% wt./vol, from about 0.15% wt./vol to about 0.45% wt./vol, from about 0.15% wt./vol to about 0.40% wt./vol, from about 0.15% wt./vol to about 0.35% wt./vol, from about 0.15% wt./vol to about 0.30% wt./vol, from about 0.15% wt./vol to about 0.25% wt./vol, or from about 0.15% wt./vol to about 0.20% wt./vol, of the overall aqueous content of the fluid reservoir.

In embodiments comprising hypochlorous acid, the concentration of the hypochlorous acid may be from about 0.20% wt./vol to about 0.50% wt./vol, from about 0.20% wt./vol to about 0.45% wt./vol, from about 0.20% wt./vol to about 0.40% wt./vol, from about 0.20% wt./vol to about 0.35% wt./vol, from about 0.20% wt./vol to about 0.30% wt./vol, or from about 0.20% wt./vol to about 0.25% wt./vol, of the overall aqueous content of the fluid reservoir.

In embodiments comprising hypochlorous acid, the concentration of the hypochlorous acid may be from about 0.25% wt./vol to about 0.50% wt./vol, from about 0.25% wt./vol to about 0.45% wt./vol, from about 0.25% wt./vol to about 0.40% wt./vol, from about 0.25% wt./vol to about 0.35% wt./vol, or from about 0.25% wt./vol to about 0.30% wt./vol, of the overall aqueous content of the fluid reservoir.

In embodiments comprising hypochlorous acid, the concentration of the hypochlorous acid may be from about 0.30% wt./vol to about 0.50% wt./vol, from about 0.30% wt./vol to about 0.45% wt./vol, from about 0.30% wt./vol to about 0.40% wt./vol, or from about 0.30% wt./vol to about 0.35% wt./vol, of the overall aqueous content of the fluid reservoir.

In embodiments comprising hypochlorous acid, the concentration of the hypochlorous acid may be from about 0.35% wt./vol to about 0.50% wt./vol, from about 0.35% wt./vol to about 0.45% wt./vol, or from about 0.35% wt./vol to about 0.40% wt./vol, of the overall aqueous content of the fluid reservoir.

In embodiments comprising hypochlorous acid, the concentration of the hypochlorous acid may be from about 0.40% wt./vol to about 0.50% wt./vol, or from about 0.40% wt./vol to about 0.45% wt./vol, of the overall aqueous content of the fluid reservoir.

In embodiments comprising hypochlorous acid, the concentration of the hypochlorous acid may be from about 0.45% wt./vol to about 0.50% wt./vol, of the overall aqueous content of the fluid reservoir.

In some embodiments, there is provided a packaged intermittent urinary catheter comprising: a single use catheter; a packaging surrounding the catheter; and a fluid reservoir holding a lubricant which, upon rupturing the fluid reservoir, is transferred into the packaging to immerse at least a portion of the catheter in the lubricant to prepare the catheter for use, wherein the lubricant comprises an antimicrobial medium, further wherein the antimicrobial medium comprises an aqueous chlorine solution comprising at least one chlorine-containing species, wherein the at least one chlorine-containing species is hypochlorous acid, and the total concentration of the hypochlorous acid is of from 0.05 to 0.5% wt./vol of the overall aqueous content of the fluid reservoir.

In some embodiments, there is provided a packaged intermittent urinary catheter comprising: a single use catheter; a packaging surrounding the catheter; and a fluid reservoir holding a lubricant which, upon rupturing the fluid reservoir, is transferred into the packaging to immerse at least a portion of the catheter in the lubricant to prepare the catheter for use, wherein the lubricant comprises an antimicrobial medium, further wherein the antimicrobial medium comprises an aqueous chlorine solution comprising at least one chlorine-containing species, wherein the at least one chlorine-containing species is hypochlorous acid, and the total concentration of the hypochlorous acid is no more than 0.45% wt./vol of the overall aqueous content of the fluid reservoir.

The antimicrobial medium may not comprise an alkali metal halide salt. Such alkali metal halide salts may be, for example, sodium chloride.

In some embodiments, the at least one chlorine-containing species may be hypochlorous acid, and the antimicrobial medium may not comprise an alkali metal halide salt. In aqueous solution, hypochlorous acid may partially dissociate into anionic hypochlorite (CIO). Thus, in embodiments where the at least one chlorine-containing species is hypochlorous acid, and the antimicrobial medium does not comprise an alkali metal halide salt, any anionic hypochlorite (CIO) which is present in the antimicrobial medium does not form a metal hypochlorite salt (for example, sodium hypochlorite, if a sodium salt (e.g., sodium chloride) was present in the antimicrobial medium). Hypochlorous acid and anionic hypochlorite (CIO) possess different microbiological activity and biological safety profiles in the technical field, and hypochlorous acid is comparatively more advantageous in this regard. Thus, advantageously, in embodiments comprising hypochlorous acid and an antimicrobial medium which does
not comprise an alkali metal halide salt, metal hypochlorite salts, for example sodium chloride, are not present.

In some embodiments, there is provided a packaged intermittent urinary catheter comprising: a single use catheter; a packaging surrounding the catheter; and a fluid reservoir holding a lubricant which, upon rupturing the fluid reservoir, is transferred into the packaging to immerse at least a portion of the catheter in the lubricant to prepare the catheter for use, wherein the lubricant comprises an antimicrobial medium, further wherein the antimicrobial medium comprises an aqueous chlorine solution comprising at least one chlorine-containing species, wherein the at least one chlorine-containing species is hypochlorous acid, and the antimicrobial medium does not comprise an alkali metal halide salt.

The aqueous chlorine solution may be obtained from a dichloroisocyanurate solution or a hypochlorite solution.

The antimicrobial medium may be present in liquid, gel or solid form.

In some embodiments, there is provided a packaged intermittent urinary catheter comprising: a single use catheter; a packaging surrounding the catheter; and a fluid reservoir holding a lubricant which, upon rupturing the fluid reservoir, is transferred into the packaging to immerse at least a portion of the catheter in the lubricant to prepare the catheter for use, wherein the lubricant comprises an antimicrobial medium, wherein the antimicrobial medium is present as an antimicrobial liquid or an antimicrobial gel.

The fluid reservoir may hold between about 1 and about 12 ml, about 1.25 and about 11 ml, about 1.50 and about 10 ml, about 1.75 and about 9 ml, about 2.00 and about 8 ml, about 2.10 and about 7 ml, about 2.20 and about 6 ml, about 2.25 and about 5 ml, about 2.30 and about 5 ml, about 2.35 and about 4 ml, about 2.40 and about 3 ml, about 2.45 and about 2.75 ml, or about 2.50 ml, of lubricant.

The fluid reservoir may hold about 1.0 ml, about 1.5 ml, about 2.0 ml, about 2.5 ml, about 3.0 ml, about 3.5 ml, about 4.0 ml, about 4.5 ml, about 5.0 ml, about 5.5 ml, about 6.0 ml, about 6.5 ml, about 7.0 ml, about 7.5 ml, about 8.0 ml, about 8.5 ml, about 9.0 ml, about 9.5 ml, about 10.0 ml, about 10.5 ml, about 11.0 ml, about 11.5 ml, or about 12.0 ml, of lubricant.

The fluid reservoir may hold at least about 1.0 ml, about 1.5 ml, about 2.0 ml, about 2.5 ml, about 3.0 ml, about 3.5 ml, about 4.0 ml, about 4.5 ml, about 5.0 ml, about 5.5 ml, about 6.0 ml, about 6.5 ml, about 7.0 ml, about 7.5 ml, about 8.0 ml, about 8.5 ml, about 9.0 ml, about 9.5 ml, about 10.0 ml, about 10.5 ml, about 11.0 ml, about 11.5 ml, or at least about 12.0 ml, of lubricant.

The fluid reservoir may hold no more than about 1.0 ml, about 1.5 ml, about 2.0 ml, about 2.5 ml, about 3.0 ml, about 3.5 ml, about 4.0 ml, about 4.5 ml, about 5.0 ml, about 5.5 ml, about 6.0 ml, about 6.5 ml, about 7.0 ml, about 7.5 ml, about 8.0 ml, about 8.5 ml, about 9.0 ml, about 9.5 ml, about 10.0 ml, about 10.5 ml, about 11.0 ml, about 11.5 ml, or no more than about 12.0 ml, of lubricant.

The volume of lubricant which the fluid reservoir may hold is smaller than the amount of sterilisation fluid held in a fluid reservoir comprised in a conventional packaged reusable intermittent catheter. This is because a volume of sterilisation fluid will be lost each time a reusable catheter is immersed in the sterilisation fluid and withdrawn from the fluid ready for re-use. As such, typically, packaged reusable intermittent catheters comprise an amount of sterilisation fluid greater than about 12 ml. Advantageously, in embodiments comprising a fluid reservoir holding an amount of lubricant of between about 1 and about 12 ml, the catheter of the present invention can be at least partially coated in the lubricant and this amount lubricant is sufficient to provide an antimicrobial effect when inserted into the urethra of a user.

In some embodiments, the catheter comprises a hollow tubular body, preferably a hollow polymeric tubular body. The hollow polymeric tubular body may comprise a base polymer.

In some embodiments, the catheter is at least partially coated with at least one additive, preferably at least one lubricious additive. Preferably, the hollow tubular body comprises at least one additive.

Additives allow for improved catheter lubricity and ease of insertion and removal. However, the use of additives on a catheter, particularly on a surface thereof, provides the catheter with complex surface chemistry which can make catheter sterilisation and repeated lubrication challenging. Such catheters have, for instance, been known to swell upon wetting during sterilisation. Such catheters are also known to suffer from dry-out, which can result in considerable changes to the catheter surface morphology. As a result, catheters can become rough and sticky, and much more prone to additive delamination. However, an antimicrobial medium comprising the chlorinebased species defined above allows for effective catheter sterilisation and lubrication, which allows for prolonged effects of the additives. Accordingly, such an antimicrobial medium allows for safe and simple catheter use, with excellent performance in relation to reducing pain and discomfort.

The at least one additive may be a hydrophilic additive.

At least one hydrophilic additive may be independently selected from the group comprising: a polyalkylene glycol, hyaluronic acid, chondroitan sulfate, chitosan, glucosaminoglucans, dextran, dextrin, dextran sulfate, cellulose acetate, carboxymethyl cellulose, hydroxyethyl cellulose, cellulosics, polypeptides, poly(2-hydroxyethyl methacrylate), polyacrylamide, polyacrylimide, poly(ethylene amine), poly(allyl amine), poly(vinyl pyrrolidone) (PVP), poly(vinyl alcohol), poly(acrylic acid), poly(methacrylic acid), acrylic acid copolymers, methacrylic acid copolymers, polyvinyl alkyl ethers, non-ionic tetrafunctional block-copolymer surfactants, gelatin, collagen, albumin, chitin, heparin, elastin, fibrin, and combinations thereof.

In some embodiments, at least one hydrophilic additive is independently selected from the group comprising: poly(ethylene glycol), poly(ethylene oxide), polypropylene glycol), poly(ethylene oxide-co-propylene oxide), poly(trimethylene glycol), poly(tetramethylene glycol), and combinations thereof.

The at least one hydrophilic additive may comprise PVP or a derivative thereof.

The at least one additive may be an amphiphilic additive. The amphiphilic additive comprises a hydrophobic portion and a hydrophilic portion. In cases where the base polymer is hydrophobic or generally hydrophobic, such as a polyolefin, the amphiphilic additive will diffuse towards and to an outer surface of the catheter body due to incompatibility of the hydrophilic portion of the amphiphilic additive with the hydrophobic base polymer. Amphiphilic additives further allow a hydration layer to be created through temporary hydrogen bonds on the catheter surface with water molecules and the chlorine-based species. As such, the catheter is able to remain sterile and lubricated for long periods of time and minimal to no changes to surface morphology are seen during sterilisation/lubrication and during catheter dry-out.

In some embodiments, the catheter comprises a hollow polymeric tubular body comprising a base polymer and an amphiphilic lubricious additive.

In some embodiments, at least one additive is polymeric or oligomeric.

At least one additive may be an A-B block copolymer comprising a hydrophobic hydrocarbon A-block and a hydrophilic B-block. In some embodiments, one or both of the hydrophobic hydrocarbon A-block and the hydrophilic B-block may be branched. The hydrophobic A-block may comprise hydrophobic hydrocarbon chains branching therefrom. The hydrophobic hydrocarbon chains may be of shorter chain lengths than the hydrophobic hydrocarbon A-block. The hydrophilic B-block may comprise further hydrophilic B-blocks branching therefrom.

In some embodiments, the additive is a B-A-B tri-block copolymer comprising a hydrophobic hydrocarbon A-block and hydrophilic B-blocks.

In other embodiments, the additive is a graft copolymer. The graft copolymer may comprise a hydrophobic hydrocarbon A-block with hydrophilic B-blocks branching therefrom. Alternatively, the graft copolymer may comprise a hydrophilic portion with hydrophobic portions branching therefrom.

In further embodiments, the additive is a brush copolymer. The additive may comprise a single hydrophilic B-block with more than one hydrophobic A-block branching from an end thereof. Alternatively, the additive may comprise a single hydrophobic A-block with more than one hydrophilic B-block branching from an end thereof. In the respective embodiments, the B-block or A-block may comprise 2, 3, 4, 5, 6, 7, 8, 9, 10, or more hydrophobic A-blocks or hydrophilic B-blocks branching from the end thereof.

In further embodiments, the additive is a star-block or multi-block copolymer comprising hydrophilic and hydrophobic monomer units.

In preferred embodiments, the additive is an A-B block copolymer comprising a hydrophobic A-block and a hydrophilic B-block.

Statements of invention below relating to the additive or a part thereof may be applied mutatis mutandis to each of the copolymer forms above.

In some embodiments, the B-block is a hydrophilic oligomer comprising at least 1, 2, 3, 4, or at least 5 monomer units. In some embodiments, the B-block comprises no greater than 20, 19, 18, 17, 16, 15, 14, 13, 12, 11, 10, 9, 8, 7, or no greater than 6 monomer units. In some embodiments, the B-block comprises between 2 and 15 monomer units, preferably between 2 and 10 monomer units. At least one monomer unit may be selected from the group comprising: alkylene oxides, alkylene glycols, epihalohydrins, unsaturated carboxylic acids, alkylene imines, lactones, vinyl alcohol, and vinyl alkanoates. At least one monomer unit may be preferably selected from the group comprising: ethylene oxide, propylene oxide, ethylene glycol, propylene glycol, epichlorohydrin, acrylic acid, methacrylic acid, ethylene imine, caprolactone, vinyl alcohol, and vinyl acetate. In some embodiments, at least one monomer unit comprises alkylene oxide groups independently selected from ethylene oxide and propylene oxide, and in preferred embodiments, all of the monomer units are ethylene oxide or all of the monomer units are propylene oxide.

The hydrophobic A-block may comprise a carbon chain of at least 5 carbon atoms, or at least 10, 15, 20, 25, 30, 35, or 40 carbon atoms. The hydrophobic portion may preferably comprise a carbon chain of between 20-52 carbon atoms.

In some embodiments, the A-block comprises a hydrocarbon chain block of the formula CH3CH2 (CH2CH2) a. The value of “a” may be between 5-25; for instance, “a” may be 5, 6, 7, 8, 9, 10, 11, 12, 13, 14, 15, 16, 17, 18, 19, 20, 21, 22, 23, 24, or 25, or a half integer of any of the above values. The value of “a” may preferably be between 9-25; for instance, “a” may be 9, 10, 11, 12, 13, 14, 15, 16, 17, 18, 19, 20, 21, 22, 23, 24, or 25, or a half integer of any of the above values.

In some embodiments, the additive is homogenously distributed with the polymer. The additive may be uniformly distributed throughout the base polymer of the catheter body.

At least some of the additive may be at or on the outer surface of the body. By “at the outer surface”, it is meant that at least a portion of the additive forms part of the surface or protrudes from the surface. In some embodiments, part of the additive is retained or anchored in the body while part of the additive forms part of or protrudes from the outer surface of the body. At least part of the hydrophilic portion of the additive may protrude from or form part of the outer surface of the body, while at least part of the hydrophobic portion may be retained or anchored within the body.

The outer surface may comprise at least one member of the group comprising: the external-facing surface of the body, the lumen of the body and any eyelets present on the body. In preferred embodiments the outer surface is the external-facing surface of the body and/or the inner lumen. In some embodiments, the outer surface may comprise the external-facing surface of the body of the catheter, the inner lumen, and the eyelets.

The additive may be concentrated at or on the outer surface of the body. For example, at least 25%, 30%, 35%, 40%, 45%, 50%, 55%, 60%, 65%, 70%, 75%, 80%, 85%, 90%, or at least 95% of the number of molecules of the additive may be at or on the outer surface of the body.

In some embodiments, at least 25%, 30%, 35%, 40%, 45%, 50%, 55%, 60%, 65%, 70%, 75%, 80%, 85%, 90%, or at least 95% of the number of molecules of additive may have hydrophilic portions that are at or on the outer surface of the body.

In some embodiments, the additive is located at and/or on at least 50, 55, 60, 65, 70, 75, 80, 85, 90, 95, 96, 97, 98 or at least 99% of the outer surface area of the polymeric tubular body, preferably at least 75% or at least 90% of the outer surface area of the polymeric tubular body or between 75% and 100% of the outer surface area.

In some embodiments, the additive is present at a concentration of at least 0.1, 0.2, 0.3. 0.4. 0.5, 0.75, 1, 2, 3, 4, 5, 10, 15 or at least 20% by weight of the combination of base polymer and additive. The additive may be present a concentration of between 0.1-20%, and more preferably between 0.5-15% or 0.5-5% by weight of the combination of base polymer and additive.

In some embodiments, the additive comprises a layer that is on or that comprises a surface of the body, preferably the outer surface.

The layer comprising the additive may be on the surface of the body. In some embodiments, the layer comprising the additive is substantially separate from the body and the layer may be bonded to the body. The layer may be bonded to the body via covalent bonds, ionic bonds, hydrogen bonds, or Van der Waals forces. The additive may be bonded to the body via one or more surface linker groups which may be present on the additive, the body of the catheter or both.

In some embodiments, the layer comprising the additive may comprise the surface of the body. In such embodiments the layer may form the surface of the body. The layer may comprise a co-extruded layer which is melded with or is physically entangled with the body, and this may form an integral layer. The layer of additive may be integrally formed with the body.

In some embodiments, polymer diffusion occurs between the layer comprising the additive and the catheter body. The layer and the body may be held together by polymer chains extending across the interface between the layer and body. In some embodiments, the additive infiltrates the catheter body

In some embodiments, the layer comprising the additive comprises or is on an inner surface of the body, an outer surface of the body, or both. The inner surface of the body may comprise a lumen of the catheter. In preferred embodiments, the layer comprising the additive comprises or is on at least an outer surface of the body.

In some embodiments, the layer comprising the additive is on or comprises at least 50, 55, 60, 65, 70, 75, 80, 85, 90, 95, 96, 97, 98 or at least 99% of the or each surface area of the body, preferably at least 75% or at least 90% of the or each surface area or between 75% and 100% of the or each surface area. In embodiments in which the layer comprising the additive comprises or is on both an inner and outer surface of the body, the additive may comprise at least 50, 55, 60, 65, 70, 75, 80, 85, 90, 95, 96, 97, 98 or at least 99% of each surface area of the body, preferably at least 75% or at least 90% of each surface area or between 75% and 100% of each surface area of both surfaces.

In some embodiments, at least 75% of the layer comprising the additive, or at least 76, 77, 78, 79, 80, 81, 82, 83, 84, 85, 86, 87, 88, 89, 90, 91, 92, 93, 94, 95, 96, 97, 98, 99, or 100% of the layer is the additive.

In some embodiments, the layer comprising the additive has an additive concentration of at least 0.1, 0.2, 0.3, 0.4, 0.5, 0.75, 1, 2, 3, 4, 5, 10, 15 or at least 20% by weight of the combination of base polymer and additive.

In some embodiments, the layer comprising the additive has an additive concentration of no greater than 70, 65, 60, 65, 60, 55, or of no greater than 50% by weight of the combination of the base polymer and additive.

The layer comprising the additive may have an additive concentration of greater than 5% by weight of the combination of the base polymer and additive. The layer may have an additive concentration of between 6-50% by weight of the combination of the base polymer and additive.

The layer comprising the additive may have an additive concentration of between 10-50% by weight of the combination of the base polymer and additive, or of between 15-50, 20-50, 25-50, 30-50, 35-50, 40-50, or of between 45-50% by weight of the combination of the base polymer and additive.

The layer comprising the additive may have an additive concentration of between 6-45% by weight of the combination of the base polymer and additive, or of between 6-40, 6-35, 6-30, 6-25, 6-20, 6-15, or of between 6-10% by weight of the combination of the base polymer and additive.

The layer comprising the additive may have an additive concentration of between 10-45% by weight of the combination of the base polymer and additive, or of between 15-45, 20-45, 25-45, 30-45, 35-45, 40-45, 10-40, 15-40, 20-40, 25-40, 30-40, 35-40, 10-35, 15-35, 20-35, 25-35, 30-35, 10-30, 15-30, 20-30, 25-30, 10-25, 15-25, 20-25, 10-20, 15-20, or of between 10-15% by weight of the combination of the base polymer and additive.

In some embodiments, the layer comprising the additive has a thickness of at least 1 pm, or of at least 2, 3, 4, 5, 6, 7, 8, 9, 10, 15, 20, 25, 30, 35, 40, 45, or of at least 50 pm.

In some embodiments, the layer comprising the additive has a thickness of no more than 10000 pm, or of no more than 9000, 8000, 7000, 6000, 5000, 4000, 3000, 2000, 1000, 900, 800, 700, 600, 500, 400, or of no more than 300 pm.

In some embodiments, the layer comprising the additive has a thickness of between 50-300 pm.

The layer comprising the additive may have a thickness of between 60-300 pm, or of between 80-300, 100-300, 120-300, 140-300, 160-300, 180-300, 200-300, 220-300, 240-300, 260-300. or of between 280-300 pm.

The layer comprising the additive may have a thickness of between 50-280 pm, or of between 50-260, 50-240, 50-220, 50-200, 50-180, 50-160, 50-140, 50-120, 50-100, 50-80, or of between 50-60 pm.

The layer comprising the additive may have a thickness of between 60-280 pm, or of between 80-280, 100-280, 120-280, 140-280, 160-280, 180-280, 200-280, 220-280, 240-280, 260-280, 60-260, 80-260, 100-260, 120-260, 140-260, 160-260, 180-260, 200-260, 220-260, 240-260, 60-240, 80-240, 100-240, 120-240, 140-240, 160-240, 180-240, 200-240, 220-240, 60-220, 80-220, 100-220, 120-220, 140-220, 160-220, 180-220, 200-220, 60-200, 80-200, 100-200, 120-200, 140-200, 160-200, 180-200, 60-180, 80-180, 100-180, 120-180, 140-180, 160-180, 60-160, 80-160, 100-160, 120-160, 140-160, 60-140, 80-140, 100-140, 120-140, 60-120, 80-120, 100-120, 60-100, 80-100, or of between 60-80 pm.

In preferred embodiments, the catheter base polymer is hydrophobic or partly hydrophobic. A hydrophobic base polymer facilitates increased hydrophobichydrophobic interactions between the hydrophobic portion of the additive and the base polymer. This further decreases the energetic favourability for the hydrophobic portion to leave the base polymer and migrate out into the more hydrophilic external environment.

In some embodiments, the base polymer comprises a polymer selected from the group consisting of: polyvinyl chloride, polytetrafluoroethylene, polyolefins, latex, silicones, synthetic rubbers, polyurethanes, polyesters, polyacrylates, polyamides, thermoplastic elastomeric materials, styrene block copolymers, polyether block amide, thermoplastic vulcanizates, thermoplastic copolyesters, thermoplastic polyamides, styrene-butadiene copolymer (SBC), styrene-ethylene-butylene-styrene copolymer (SEBS), and water disintegrable or enzymatically hydrolysable material, or combinations, blends or copolymers of any of the above materials.

In preferred embodiments, the base polymer comprises a polymer selected from the group consisting of: polyolefins, polyesters, poly acrylates, polyamides, thermoplastic elastomeric material, polyether block amide, thermoplastic vulcanizates, thermoplastic copolyesters, thermoplastic polyamides, fluororubber, and water disintegrable or enzymatically hydrolysable material or combinations, blends or copolymers of any of the above materials.

In some embodiments, said water disintegrable or enzymatically hydrolysable material comprises a material of the group consisting of: polyvinyl alcohol, extrudable polyvinyl alcohol, polyacrylic acids, polylactic acid, polyesters, polyglycolide, polyglycolic acid, poly lactic-co-glycolic acid, polylactide, amines, polyacrylamides, poly(N-(2-Hydroxypropyl) methacrylamide), starch, modified starches or derivatives, amylopectin, pectin, xanthan, scleroglucan, dextrin, chitosans, chitins, agar, alginate, carrageenans, laminarin, saccharides, polysaccharides, sucrose, polyethylene oxide, polypropylene oxide, acrylics, polyacrylic acid blends, poly(methacrylic acid), polystyrene sulfonate, polyethylene sulfonate, lignin sulfonate, polymethacrylamides, copolymers of aminoalkyl-acrylamides and methacrylamides, melamine-formaldehyde copolymers, vinyl alcohol copolymers, cellulose ethers, poly-ethers, polyethylene oxide, blends of polyethylene-polypropylene glycol, carboxymethyl cellulose, guar gum, locust bean gum, hydroxypropyl cellulose, vinylpyrrolidone polymers and copolymers, polyvinyl pyrrolidone-ethylene-vinyl acetate, polyvinyl pyrrolidone-carboxymethyl cellulose, carboxymethyl cellulose shellac, copolymers of vinylpyrrolidone with vinyl acetate, hydroxyethyl cellulose, gelatin, poly-caprolactone, poly(p-dioxanone), or combinations, blends or co-polymers of any of the above materials.

In other preferred embodiments, the base polymer comprises a polymer selected from the group consisting of: polyolefins, polyvinyl chloride, polyurethane, styrenebutadiene copolymer (SBC), styrene-ethylene-butylene-styrene copolymer (SEBS), and thermoplastic elastomeric material or combinations, blends or copolymers of any of the above materials.

In some preferred embodiments, the base polymer comprises a polyolefin, especially polyethylene and/or polypropylene.

In some preferred embodiments, the base polymer comprises a thermoplastic elastomeric material. The base polymer may comprise a thermoplastic polyolefin.

The thermoplastic base polymer may comprise a hydrophobic polymer selected from the group consisting of: Accurel™, Styroflex™, Styrolux™, MelifleX™, and Mediprene™ and any combination thereof.

The thermoplastic base polymer may comprise Estane™ 58315, which is both hydrophobic and hydrophilic.

The intermittent urinary catheter is typically inserted into a body for short time periods, such as less than a day.

In some embodiments, the medium comprises at least one hypochlorite salt. The at least one hypochlorite salt may preferably comprise a countercation. The countercation may comprise an inorganic countercation. The countercation may comprise a metal countercation. In some embodiments, at least one countercation is independently selected from the group consisting of: an alkali metal cation, an alkaline earth metal cation, a group III metal cation, a transition metal cation, an ammonium cation, an aromatic nitrogen-based cation, and combinations thereof. At least one countercation may be independently selected from the group consisting of: ammonium, calcium, iron, magnesium, potassium, pyridinium, quaternary ammonium, sodium, copper, aluminium, lithium, beryllium, strontium, and zinc. At least one countercation may preferably be an alkali metal cation or an alkaline earth metal cation. At least one countercation may preferably be independently selected from the group consisting of: calcium, lithium, and sodium. In preferred embodiments, at least one hypochlorite salt is sodium hypochlorite.

In some embodiments, the medium comprises hypochlorous acid and at least one hypochlorite salt. At least one hypochlorite salt may preferably be as described in statements of invention above. The medium may preferably comprise hypochlorous acid and at least one alkali metal hypochlorite salt. In some embodiments, the medium comprises hypochlorous acid and sodium hypochlorite.

In embodiments wherein the medium comprises hypochlorous acid and at least one hypochlorite salt, the pH of the medium may preferably be between 6-9.

In some embodiments, the medium comprises chlorine dioxide and at least one of: hypochlorous acid, at least one chlorhexidine salt, and at least one hypochlorite salt. In some embodiments, the medium comprises chlorine dioxide, hypochlorous acid, at least one chlorhexidine salt, and at least one hypochlorite salt. In such embodiments, at least one hypochlorite salt may preferably be as described in statements of invention above. At least one hypochlorite salt may preferably be an alkali metal hypochlorite salt, which may comprise sodium hypochlorite. The at least one chlorhexidine salt may be chlorhexidine digluconate, chlorhexidine diacetate, or combinations thereof.

In some embodiments, the antimicrobial medium is present as a liquid having a viscosity of greater than 0.5 cP, or of greater than 1, 1.5, 2, 3, 4, 5, 10, 20, 30, 40, 50, 75, 100, 150, 200, 300, 400, 500, 600, 700, 800, 900, or of greater than 1000 cP. The antimicrobial medium may have a viscosity of no greater than 100000 cP, or of no greater than 90000, 80000, 70000, 60000, 50000, 40000, 30000, 20000, 10000, 5000, 4000, 3000, 2000, 1000, 500, 400, 300, 200, 100, 50, 25, 10, 5, 4, 3, 2, or of no greater than 1 cP. The antimicrobial medium may have a viscosity of between 0.5-5000 cP, or of between 0.5-2500, 0.5-1000, 1-1000, 10-1000, 50-1000, 100-1000, 500-1000, 0.5-500, 1-500, 10-500, 50-500, 100-500, 250-500, 0.5-250, 1-250, 10-250, 50-250, or of between 100-250 cP.

The antimicrobial medium may be a catheter wetting agent. The antimicrobial medium may encourage hydrophilic portions of lubricating additives within the catheter to seek towards an outer surface of the catheter, which further enhances the lubricating effect of the additive.

In some embodiments, the antimicrobial medium has a pH of at least 2, 2.5, 3, 3.5, 4, 4.5, 5, 5.5, 6, 6.5, 7, 7.5, 8, 8.5, or of no greater than 9. In some embodiments, the antimicrobial medium may have a pH of no greater than 14, or of no greater than 13.5, 13, 12.5, 12, 11.5, 11, 10.5, or of no greater than 10.

In some embodiments, the antimicrobial medium has a pH of between 2-14, 3-13, or of between 4-12.

In some embodiments, the antimicrobial medium comprises at least one further species that is independently selected from the group consisting of: a chlorine-containing bleaching and/or oxidising agent, a peroxide species, a base, an acid, a photosensitiser, a permanganate species, an alcohol, a phenol, an aldehyde, ionic silver, molecular iodine or an iodophor, an imine-containing species, a salt and combinations thereof.

In some embodiments, the antimicrobial medium comprises at least one further chlorine-containing bleaching and/or oxidising agent that is independently selected from the group consisting of: molecular chlorine, a dichloroisocyanurate salt, chloroazodin, dichlorodimethylhydantoin, chloroxylenol, chlorhexidine, and combinations thereof.

In some embodiments, the total concentration of chlorine-containing bleaching and/or oxidising agents in the antimicrobial medium is between 0.05-0.6 wt. % of the antimicrobial medium, or between 0.01-0.5, 0.015-0.4, 0.02-0.3, or between 0.025-0.2 wt. % of the antimicrobial medium, or between 0.025-0.1 wt. %.

The antimicrobial medium may comprise at least one further salt (in addition to any hypochlorite salt present). At least one salt may comprise a cation that is independently selected from the group consisting of: ammonium, calcium, iron, magnesium, potassium, pyridinium, quaternary ammonium, sodium, copper, aluminium, lithium, beryllium. strontium, and zinc. At least one salt may preferably comprise an alkali metal cation. At least one salt may comprise an anion that is independently selected from the group consisting of: acetate, carbonate, bicarbonate, chloride, citrate, glutamate, fluoride, bromide, iodide, nitrate, nitrite, oxide, phosphate, ferrocyanide, silicate, gluconate, and sulfate. At least one salt may preferably comprise a halide anion.

At least one of the further salts may be independently selected from the group consisting: sodium chloride, potassium chloride, calcium chloride, magnesium chloride, calcium chloride, sodium nitrite, magnesium nitrate, calcium nitrate, potassium carbonate, sodium carbonate, sodium bicarbonate, potassium iodide, copper iodide, sodium ferrocyanide, monosodium glutamate, calcium silicate, sodium citrate, potassium citrate, sodium phosphate, potassium phosphate, sodium sulfate, calcium sulfate, sodium gluconate, calcium gluconate, potassium gluconate, sodium acetate, and potassium acetate.

At least one of the further salts may preferably be an alkali metal halide. At least one salt may be independently selected from the group consisting of: an alkali metal chloride, an alkali metal bromide, an alkali metal iodide, and combinations thereof.

Preferably, the antimicrobial medium may comprise an alkali metal salt and a hypochlorite salt. The medium may comprise a halide salt and a hypochlorite salt. The antimicrobial medium may comprise an alkali metal halide and a hypochlorite salt.

In a particularly preferred embodiment, the antimicrobial medium comprises sodium chloride. The antimicrobial medium may preferably comprise a hypochlorite salt and sodium chloride, preferably an alkali metal hypochlorite and sodium chloride, and more preferably sodium hypochlorite and sodium chloride.

In some embodiments, the total concentration of further salt in the antimicrobial medium is between 5-30 wt. %, or of between 10-25, or of between 10-20 wt. % of the antimicrobial medium.

In some embodiments, the antimicrobial medium comprises the hypochlorite salt in a total concentration of between 0.25-2 wt. %, or 0.5-1.5 wt. % of the antimicrobial medium, and the further salt in a total concentration of between 5-30, 10-25, or between 10-20 wt. % of the antimicrobial medium.

In some embodiments, the ratio of the total concentration of further salt to hypochlorite salt in the antimicrobial medium is at least 5, or at least 10, or at least 15. The ratio of the total concentration of further salt to hypochlorite salt in the antimicrobial medium may be between 5-30, or between 10-25, or between 10-20.

In some embodiments, the antimicrobial medium further comprises at least one base.

At least one base may comprise an inorganic base. At least one inorganic base may be independently selected from the group consisting of: a hydroxide base, a carbonate base, a bicarbonate base, and combinations thereof. In some preferred embodiments, at least one base is a hydroxide base. At least one hydroxide base may be independently selected from the group consisting of: an alkali metal hydroxide, an alkaline earth metal hydroxide, a group III metal hydroxide, a transition metal hydroxide, and combinations thereof. At least one hydroxide base may preferably comprise an alkali metal hydroxide and/or an alkaline earth metal hydroxide. In some embodiments, at least one base comprises an alkali metal hydroxide that is independently selected from the group comprising: lithium hydroxide, sodium hydroxide, potassium hydroxide, and combinations thereof.

Preferably, the antimicrobial medium may comprise an alkali metal base and a hypochlorite salt. The antimicrobial medium may comprise a hydroxide base and a hypochlorite salt. The antimicrobial medium may comprise an alkali metal hydroxide and a hypochlorite salt.

In a particularly preferred embodiment, the base comprises sodium hydroxide. The antimicrobial medium may preferably comprise a hypochlorite salt and sodium hydroxide, preferably an alkali metal hypochlorite and sodium hydroxide, and more preferably sodium hypochlorite and sodium hydroxide.

In some embodiments, the antimicrobial medium comprises at least one acid that is a carboxylic acid. In some embodiments, the antimicrobial medium comprises at least one acid that is independently selected from the group consisting of: acetic acid, citric acid, peracetic acid, diperoxy dodecanoic acid, and combinations thereof.

In some embodiments, the antimicrobial medium comprises at least one Cl—CIO alcohol, or at least one C1-C5 alcohol, or at least one C1-C3 alcohol. In some embodiments, the antimicrobial medium comprises at least one alcohol that is independently selected from the group consisting of: ethanol, isopropanol, and combinations thereof.

In some embodiments, the antimicrobial medium comprises at least one phenol that is independently selected from the group consisting of: phenol, thymol, chloroxylenol, and combinations thereof.

In some embodiments, the antimicrobial medium comprises at least one aldehyde that is independently selected from the group consisting of: glutaraldehyde, noxytiolin, and combinations thereof.

In some embodiments, the antimicrobial medium comprises at least one imine-containing species that is independently selected from: polyhexanide, octenidine, and combinations thereof.

The additive may be activated upon contact with the aqueous chlorine solution.

In some embodiments, the fluid reservoir is pierceable, in use, to release the contained lubricant from the fluid reservoir and into direct contact with the catheter in the packaging surrounding the catheter, preferably without requiring opening of the packaging.

Prior to opening the packaging, prior to removing the catheter and/or prior to inserting the catheter, the user may release the lubricant from the fluid reservoir and apply the lubricant to the outer surface of the catheter.

In some embodiments, the catheter is a catheter which does not comprise an amphiphilic additive. Such catheters typically require application of a lubricious medium to the surface of the catheter prior to insertion into a user.

In some embodiments the catheter is an intermittent catheter which comprises an amphiphilic additive. The catheter may comprise a hollow polymeric tubular body which is compounded with the amphiphilic additive. Advantageously, as the base polymer of the catheter is hydrophobic or generally hydrophobic, the amphiphilic additive will diffuse towards and to an outer surface of the catheter body due to incompatibility of the hydrophilic portion of the amphiphilic additive with the hydrophobic base polymer. The amphiphilic additive allows a hydration layer to be created through temporary hydrogen bonds on the catheter surface with water molecules and any chlorine-based species present when the catheter is wetted, such that the surface remains lubricious for longer periods which aids user comfort during insertion and withdrawal of the catheter.

According to a second aspect of the invention, there is provided a method of using a packaged intermittent urinary catheter including:
a. Providing a single use catheter in a sealed packaging surrounding the catheter;
b. Providing a fluid reservoir within the packaging, the fluid reservoir holding a lubricant comprising an antimicrobial medium; c. Rupturing the fluid reservoir to transfer the lubricant into the packaging to immerse at least a portion of the catheter in the lubricant;
d. Forming an opening in the packaging; and
c. Removing the catheter from the packaging through the opening.

The packaged intermittent urinary catheter of the second aspect is preferably the packaged intermittent urinary catheter of the first aspect of the invention.

In some embodiments, step (c) comprises immersing at least 20% of the outer surface area of the catheter in the lubricant, or at least 25, 30, 35, 40, 45, 50, 55, 60, 65, 70, 75, 80, 85, 90, 95, 96, 97, 98, or at least 99% of the outer surface area of the catheter, preferably at least 75%, or at least 90% of the outer surface area, or between 75% and 100% of the outer surface area of the catheter.

In some embodiments, step (c) comprises submerging the catheter in the lubricant.

The catheter may be held in a pouch. The pouch may comprise the fluid reservoir. The pouch may comprise the catheter at least partially immersed in the lubricant. In embodiments comprising the catheter and fluid reservoir each held in a pouch, the method may further comprise a step after step (d) of forming an opening in the pouch and removing the catheter from the pouch. The pouch may be formed of flexible liquid impermeable material. Beneficially, the flexible liquid impermeable material may provide a sterile environment for the catheter prior to use.

Submerging the catheter in the lubricant has been shown to result in optimal catheter lubricity.

In some embodiments, step (c) comprises immersing at least a portion of the catheter in the lubricant for a total time of at least 5 seconds, or at least 10, 20, 30, 40, or at least 50 seconds, or at least 1 minute, or at least 2, 3, 4, or at least 5 minutes, or at least 10, 20, 30, 40, or at least 50 minutes, or at least 1 hour, or at least 1.5, 2, 2.5, 3, 3.5, or at least 4 hours. Step (c) may comprise immersing the catheter in the lubricant for a total time of no greater than 1 week, or no greater than 6 days, or no greater than 5, 4, 3, 2, or no greater than 1 day, or no greater than 20 hours, 19, 18, 17, 16, 15, 14, 13, 12, 11, 10, 9, 8, 7, 6, or no greater than 5 hours.

In some embodiments, step (c) is performed at a temperature of at least 1° C., or at least 2, 3, 4, or at least 5° C. In some embodiments, step (c) is performed at a temperature of no greater than 60° C., or no greater than 55, 50, 45, 40, 35, 30, or no greater than 25° C. In some embodiments, step (c) is performed at a temperature of between 1-60° C., or between 5-50, or between 10-40° C., or between 15-30° C.

In some embodiments, there is provided a method of using a packaged intermittent urinary catheter including:
a. Providing a single use catheter in a sealed packaging surrounding the catheter;
b. Providing a fluid reservoir within the packaging, the fluid reservoir holding a lubricant comprising an antimicrobial medium, wherein the antimicrobial medium comprises an aqueous chlorine solution, further wherein the aqueous chlorine solution comprises at least one chlorine-containing species independently chosen from: hypochlorous acid, at least one hypochlorite salt, chlorine dioxide, and combinations thereof; c. Rupturing the fluid reservoir to transfer the lubricant into the packaging to immerse at least a portion of the catheter in the lubricant;
d. Forming an opening in the packaging; and
e. Removing the catheter from the packaging through the opening.

In some embodiments, there is provided a method of using a packaged intermittent urinary catheter including:
a. Providing a single use catheter in a sealed packaging surrounding the catheter;
b. Providing a fluid reservoir within the packaging, the fluid reservoir holding a lubricant comprising an antimicrobial medium, wherein the antimicrobial medium comprises an aqueous chlorine solution, further wherein the aqueous chlorine solution comprises at least one chlorine-containing species independently chosen from: hypochlorous acid, at least one hypochlorite salt, chlorine dioxide, and combinations thereof, and the at least one chlorine-containing species is present in an amount of between 100 and 4500 ppm of the overall aqueous content of the fluid reservoir;
c. Rupturing the fluid reservoir to transfer the lubricant into the packaging to immerse at least a portion of the catheter in the lubricant;
d. Forming an opening in the packaging; and
e. Removing the catheter from the packaging through the opening.

In some embodiments, there is provided a method of using a packaged intermittent urinary catheter including:
a. Providing a single use catheter in a sealed packaging surrounding the catheter;
b. Providing a fluid reservoir within the packaging, the fluid reservoir holding a lubricant comprising an antimicrobial medium, wherein the antimicrobial medium comprises an aqueous chlorine solution, further wherein the aqueous chlorine solution comprises at least one chlorine-containing species independently chosen from: hypochlorous acid, at least one hypochlorite salt, chlorine dioxide, and combinations thereof, and the total concentration of the at least one chlorine-containing species is of from 0.05 to 0.5% wt./vol of the overall aqueous content of the fluid reservoir;
c. Rupturing the fluid reservoir to transfer the lubricant into the packaging to immerse at least a portion of the catheter in the lubricant;
d. Forming an opening in the packaging; and
e. Removing the catheter from the packaging through the opening.

In some embodiments, there is provided a method of using a packaged intermittent urinary catheter including:
a. Providing a single use catheter in a sealed packaging surrounding the catheter;
b. Providing a fluid reservoir within the packaging, the fluid reservoir holding a lubricant comprising an antimicrobial medium, wherein the antimicrobial medium comprises an aqueous chlorine solution, further wherein the aqueous chlorine solution comprises at least one chlorine-containing species independently chosen from: hypochlorous acid, at least one hypochlorite salt, chlorine dioxide, and combinations thereof, and the total concentration of the at least one chlorine-containing species is no more than 0.45% wt./vol of the overall aqueous content of the fluid reservoir;
c. Rupturing the fluid reservoir to transfer the lubricant into the packaging to immerse at least a portion of the catheter in the lubricant;
d. Forming an opening in the packaging; and
e. Removing the catheter from the packaging through the opening.

In some embodiments, there is provided a method of using a packaged intermittent urinary catheter including:
a. Providing a single use catheter in a sealed packaging surrounding the catheter;
b. Providing a fluid reservoir within the packaging, the fluid reservoir holding a lubricant comprising an antimicrobial medium, wherein the antimicrobial medium comprises an aqueous chlorine solution, further wherein the aqueous chlorine solution comprises hypochlorous acid; c. Rupturing the fluid reservoir to transfer the lubricant into the packaging to immerse at least a portion of the catheter in the lubricant;
d. Forming an opening in the packaging; and
e. Removing the catheter from the packaging through the opening.

In some embodiments, there is provided a method of using a packaged intermittent urinary catheter including:
a. Providing a single use catheter in a sealed packaging surrounding the catheter;
b. Providing a fluid reservoir within the packaging, the fluid reservoir holding a lubricant comprising an antimicrobial medium, wherein the antimicrobial medium comprises an aqueous chlorine solution, further wherein the aqueous chlorine solution comprises hypochlorous acid, and the hypochlorous acid is present in an amount of between 100 and 4500 ppm of the overall aqueous content of the fluid reservoir; c. Rupturing the fluid reservoir to transfer the lubricant into the packaging to immerse at least a portion of the catheter in the lubricant;
d. Forming an opening in the packaging; and
e. Removing the catheter from the packaging through the opening.

In some embodiments, there is provided a method of using a packaged intermittent urinary catheter including:
a. Providing a single use catheter in a sealed packaging surrounding the catheter;
b. Providing a fluid reservoir within the packaging, the fluid reservoir holding a lubricant comprising an antimicrobial medium, wherein the antimicrobial medium comprises an aqueous chlorine solution, further wherein the aqueous chlorine solution comprises hypochlorous acid, and the total concentration of the hypochlorous acid is of from 0.05 to 0.5% wt./vol of the overall aqueous content of the fluid reservoir;
c. Rupturing the fluid reservoir to transfer the lubricant into the packaging to immerse at least a portion of the catheter in the lubricant;
d. Forming an opening in the packaging; and
e. Removing the catheter from the packaging through the opening.

In some embodiments, there is provided a method of using a packaged intermittent urinary catheter including:
a. Providing a single use catheter in a sealed packaging surrounding the catheter;
b. Providing a fluid reservoir within the packaging, the fluid reservoir holding a lubricant comprising an antimicrobial medium, wherein the antimicrobial medium comprises an aqueous chlorine solution, further wherein the aqueous chlorine solution comprises hypochlorous acid, and the total concentration of the hypochlorous acid is no more than 0.45% wt./vol of the overall aqueous content of the fluid reservoir;
c. Rupturing the fluid reservoir to transfer the lubricant into the packaging to immerse at least a portion of the catheter in the lubricant;
d. Forming an opening in the packaging; and
e. Removing the catheter from the packaging through the opening.

In some embodiments, there is provided a method of using a packaged intermittent urinary catheter including:
a. Providing a single use catheter in a sealed packaging surrounding the catheter;
b. Providing a fluid reservoir within the packaging, the fluid reservoir holding a lubricant comprising an antimicrobial medium, wherein the antimicrobial medium comprises an aqueous chlorine solution, further wherein the aqueous chlorine solution comprises hypochlorous acid, and the antimicrobial medium does not comprise an alkali metal halide salt; c. Rupturing the fluid reservoir to transfer the lubricant into the packaging to immerse at least a portion of the catheter in the lubricant;
d. Forming an opening in the packaging; and
e. Removing the catheter from the packaging through the opening.

The invention according to the second aspect may of course include any one or more of the features, optional or otherwise, of the invention according to any other aspect.

According to a third aspect of the invention, there is provided a method of using a packaged intermittent urinary catheter according to the first aspect including:
a. Removing the catheter from the packaging through an opening in the packaging;
b. Performing catheterisation with the catheter; and
c. Disposing of the catheter without re-use.

The invention according to the third aspect may of course include any one or more of the features, optional or otherwise, of the invention according to any other aspect.

According to a fourth aspect of the invention, there is provided a use of a medium comprising at least one species independently chosen from: hypochlorous acid, at least one hypochlorite salt, chlorine dioxide, at least one chlorhexidine salt, and combinations thereof, as an antimicrobial medium for a single use intermittent urinary catheter.

In some embodiments, there is provided a use of a medium comprising at least one species independently chosen from: hypochlorous acid, chlorine dioxide, and combinations thereof, as an antimicrobial medium for a single use intermittent urinary catheter.

In some embodiments, there is provided a use of a medium comprising hypochlorous acid as an antimicrobial medium for a single use intermittent urinary catheter.

In some embodiments, there is provided a use of a medium comprising hypochlorous acid as an antimicrobial medium for a single use intermittent urinary catheter, wherein the hypochlorous acid is present in an amount of between 100 and 4500 ppm of the overall aqueous content of the medium.

In some embodiments, there is provided a use of a medium comprising hypochlorous acid as an antimicrobial medium for a single use intermittent urinary catheter, wherein the total concentration of the hypochlorous acid is of from 0.05 to 0.5% wt./vol of the overall aqueous content of the medium.

In some embodiments, there is provided a use of a medium comprising hypochlorous acid as an antimicrobial medium for a single use intermittent urinary catheter, wherein the total concentration of the hypochlorous acid is no more than 0.45% wt./vol of the overall aqueous content of the fluid reservoir.

In some embodiments, there is provided a use of a medium comprising hypochlorous acid as an antimicrobial medium for a single use intermittent urinary catheter, wherein the antimicrobial medium does not comprise an alkali metal halide salt.

The single use intermittent urinary catheter may be treated on at least a portion of at least one surface with the antimicrobial medium, preferably at least a portion of an outer surface.

The antimicrobial medium is preferably the antimicrobial medium of the first aspect of the invention.

The invention according to the fourth aspect may of course include any one or more of the features, optional or otherwise, of the invention according to any other aspect.

According to a fifth aspect of the invention, there is provided a packaged single use intermittent urinary catheter kit, the kit comprising a packaging comprising: a single use intermittent urinary catheter, and a fluid reservoir holding a lubricant comprising an antimicrobial medium.

In some embodiments, there is provided a packaged single use intermittent urinary catheter kit, the kit comprising a packaging comprising: a single use intermittent urinary catheter, and a fluid reservoir holding a lubricant comprising an antimicrobial medium, wherein the antimicrobial medium comprises at least one species independently chosen from: hypochlorous acid, chlorine dioxide, and combinations thereof.

In some embodiments, there is provided a packaged single use intermittent urinary catheter kit, the kit comprising a packaging comprising: a single use intermittent urinary catheter, and a fluid reservoir holding a lubricant comprising an antimicrobial medium, wherein the antimicrobial medium comprises hypochlorous acid.

In some embodiments, there is provided a packaged single use intermittent urinary catheter kit, the kit comprising a packaging comprising: a single use intermittent urinary catheter, and a fluid reservoir holding a lubricant comprising an antimicrobial medium, wherein the antimicrobial medium comprises hypochlorous acid, and the hypochlorous acid is present in an amount of between 100 and 4500 ppm of the overall aqueous content of the fluid reservoir.

In some embodiments, there is provided a packaged single use intermittent urinary catheter kit, the kit comprising a packaging comprising: a single use intermittent urinary catheter, and a fluid reservoir holding a lubricant comprising an antimicrobial medium, wherein the antimicrobial medium comprises hypochlorous acid, and the total concentration of the hypochlorous acid is of from 0.05 to 0.5% wt./vol of the overall aqueous content of the fluid reservoir.

In some embodiments, there is provided a packaged single use intermittent urinary catheter kit, the kit comprising a packaging comprising: a single use intermittent urinary catheter, and a fluid reservoir holding a lubricant comprising an antimicrobial medium, wherein the antimicrobial medium comprises hypochlorous acid, and the total concentration of the hypochlorous acid is no more than 0.45% wt./vol of the overall aqueous content of the fluid reservoir.

In some embodiments, there is provided a packaged single use intermittent urinary catheter kit, the kit comprising a packaging comprising: a single use intermittent urinary catheter, and a fluid reservoir holding a lubricant comprising an antimicrobial medium, wherein the antimicrobial medium comprises hypochlorous acid, and the antimicrobial medium does not comprise an alkali metal halide salt.

The invention according to the fifth aspect may of course include any one or more of the features, optional or otherwise, of the invention according to any other aspect.

The following statements may be applied mutatis mutandis to all aspects of the present invention.

Catheter assemblies or components thereof disclosed in one or more of the following applications, which are hereby incorporated by reference in their entirety, may be used in the packaged intermittent urinary catheter of the first aspect of the invention, the method of the second aspect of the invention, the method of the third aspect of the invention, as part of the use of the fourth aspect of the invention and/or in the kit of the fifth aspect of the invention: WO2019/123004A1, WO2019/123003A1, WO2022/223986A1, WO2022/223987A1, WO2022/223985A1. WO2022/223984A1, WO2022/223983A1, WO2022/223982A1, WO2022/223981A1, WO2022/223980AI, WO2022/223979A1, WO2022/223978A1, PCT/GB2023/052464,
PCT/GB2023/052467, PCT/GB2023/052469, PCT/GB2023/052470,
PCT/GB 2023/052693, GB2305009.9, GB2314379.5, GB2314382.9, GB2314381.1, and PCT/GB 2023/052465. In particular, the chemistry of the present invention may be used with the catheter assemblies or components thereof disclosed in the examples and figures of the above applications, or with any other devices/technologies as set out in the statements, examples and/or claims of the above applications.

Catheters, kits and components thereof marketed under the ConvaTec GentleCath (RTM) product range, including catheter kits sold under the GentleCath (RTM) Glide and GentleCath (RTM) Glide Hydrophilic ranges (comprising elongated
packaging containing a catheter and burstable sachets containing a wetting agent), may be used in the packaged intermittent urinary catheter of the first aspect of the invention, the method of the second aspect of the invention, the method of the third aspect of the invention, as part of the use of the fourth aspect of the invention and/or in the kit of the fifth aspect of the invention. In particular, the chemistry of the present invention may be used with the catheters, kits and components thereof marketed under the GentleCath (RTM) range. For instance, the medium of the invention may be used as a wetting agent with the catheter kits sold under the GentleCath (RTM) Glide and GentleCath (RTM) Glide Hydrophilic ranges.

Flip open catheter packaging or components thereof disclosed in Hollister Inc EP patent application 3445436 and U.S. Pat. Nos. 10,561,817, 11,534,573 and 11,103,676, which are hereby incorporated by reference in their entirety, may be used in the packaged intermittent urinary catheter of the first aspect of the invention, the method of the second aspect of the invention, the method of the third aspect of the invention, as part of the use of the fourth aspect of the invention and/or in the kit of the fifth aspect of the invention. In particular, the chemistry of the present invention may be used with the flip open catheter packaging or components thereof disclosed in the examples and figures of the above application and patents, or with any other devices/technologies as set out in the statements, examples and/or claims of the above application and patents.

Catheters, kits and components thereof marketed under the SpecdiCath (RTM) product range by Coloplast Ltd, including catheter kits sold under the SpeediCath (RTM) Standard range (comprising elongated packaging containing a catheter and burstable sachets containing a wetting agent, or comprising a packaging containing a catheter in direct contact with a wetting agent), may be used in the packaged intermittent urinary catheter of the first aspect of the invention, the method of the second aspect of the invention, the method of the third aspect of the invention, as part of the use of the fourth aspect of the invention and/or in the kit of the fifth aspect of the invention. In particular, the chemistry of the present invention may be used with the catheters, kits and components thereof marketed under the SpeediCath (RTM) range. For instance, the medium of the invention may be used as a wetting agent with the catheter kits sold under the SpeediCath (RTM) Standard range.

### DETAILED DESCRIPTION OF THE INVENTION

In order that the invention may be more clearly understood one or more embodiments thereof will now be described, by way of example only, with reference to the accompanying drawings, of which:

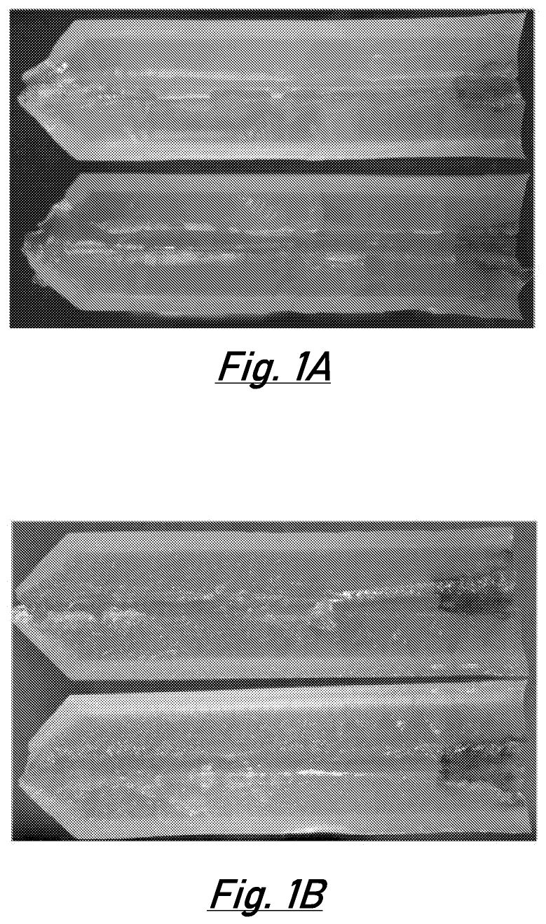

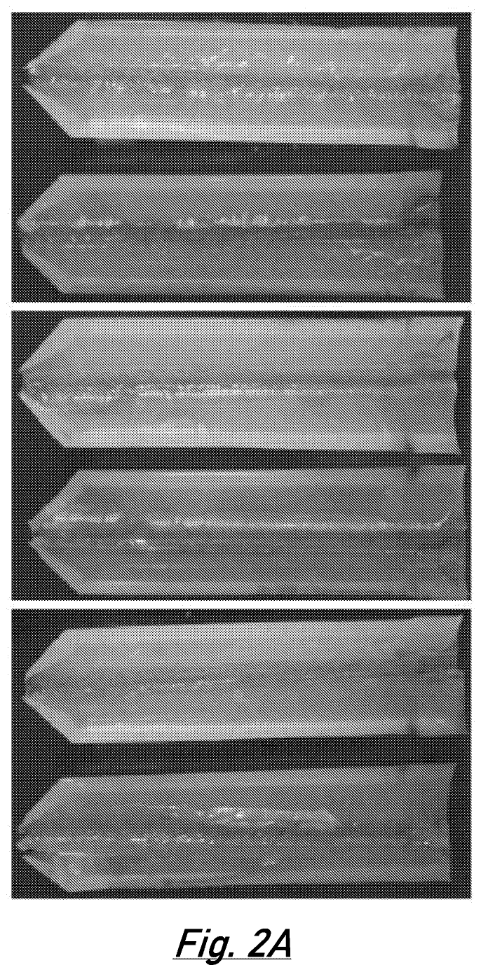

FIG. 1A is a photograph showing a visual displacement image of a Urethral Agar Channel of Example 1 A;

FIG. 1B is a photograph showing a visual displacement image of a Urethral Agar Channel of Example 1B;

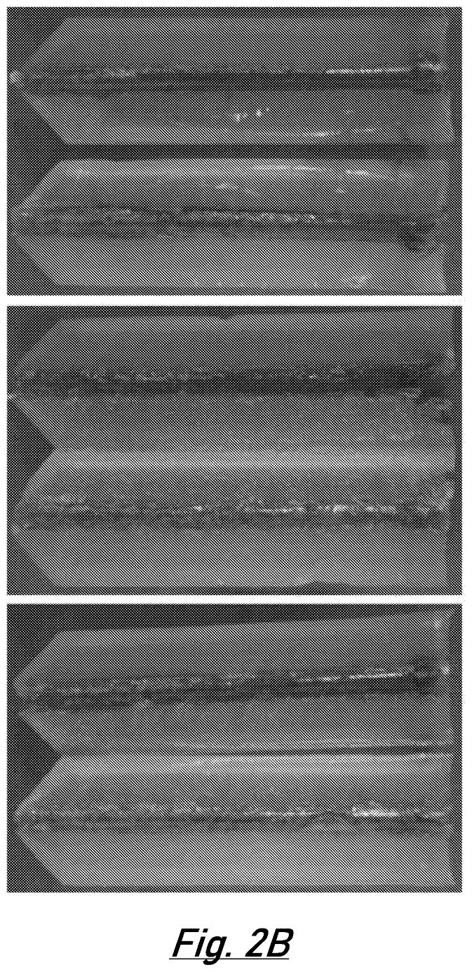

FIG. 2A is a photograph showing a visual displacement image of a Urethral Agar Channel of Example 2A;

FIG. 2B is a photograph showing a visual displacement image of a Urethral Agar Channel of Example 2B;

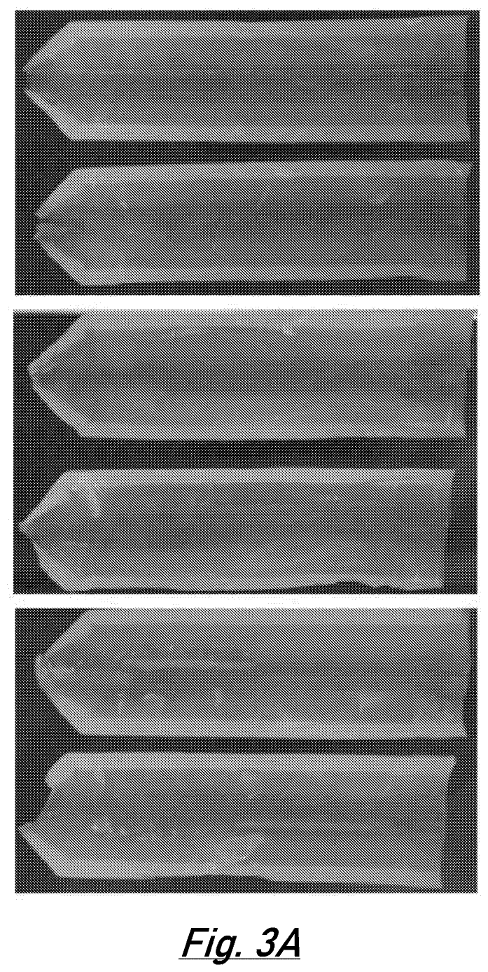

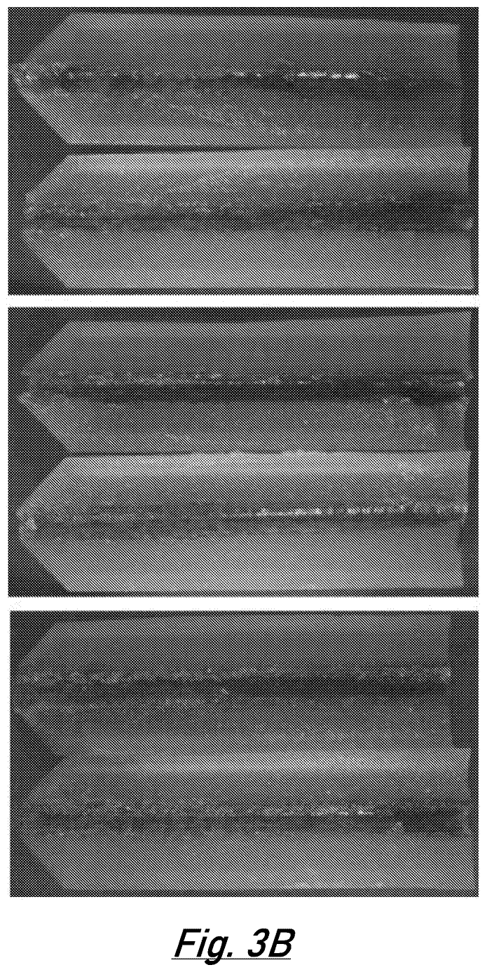

FIG. 3A is a photograph showing a visual displacement image of a Urethral Agar Channel of Example 3 A (250 ppm);

FIG. 3B is a photograph showing a visual displacement image of a Urethral Agar Channel of Example 3B (250 ppm);

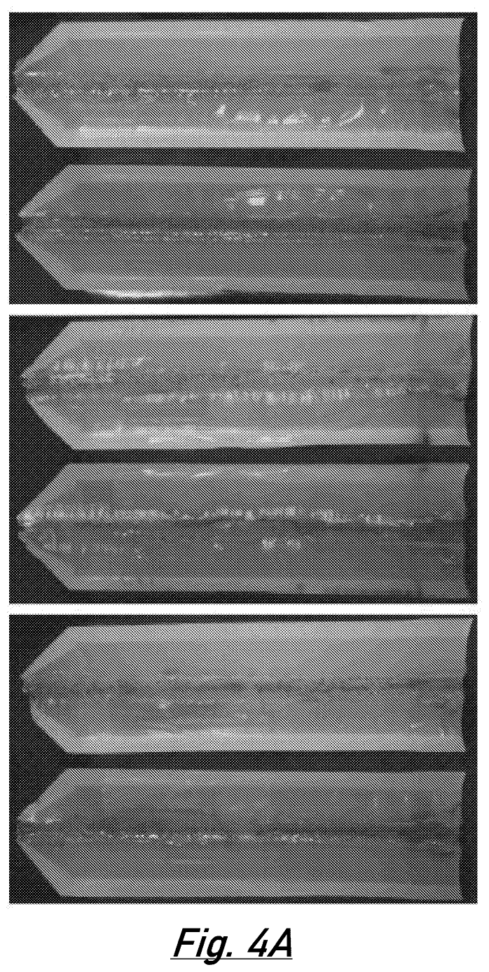

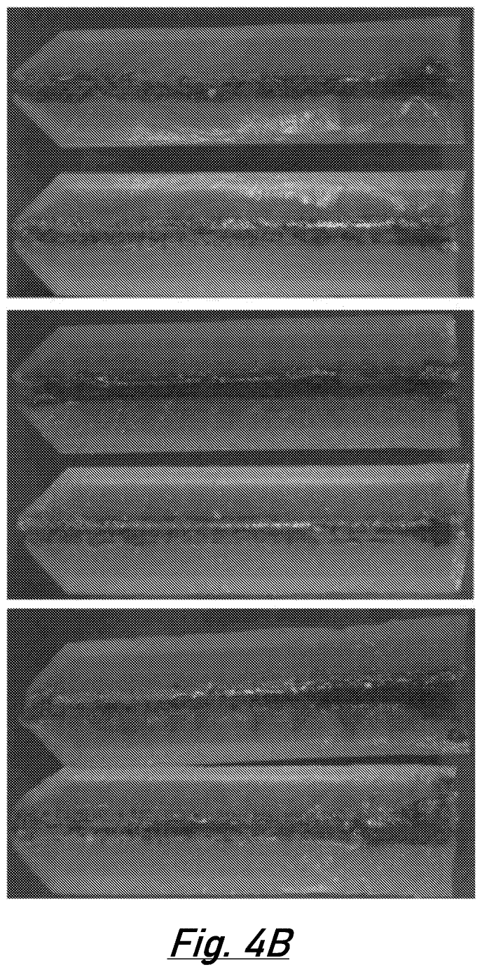

FIG. 4A is a photograph showing a visual displacement image of a Urethral Agar Channel of Example 4A (675 ppm);

FIG. 4B is a photograph showing a visual displacement image of a Urethral Agar Channel of Example 4B (675 ppm);

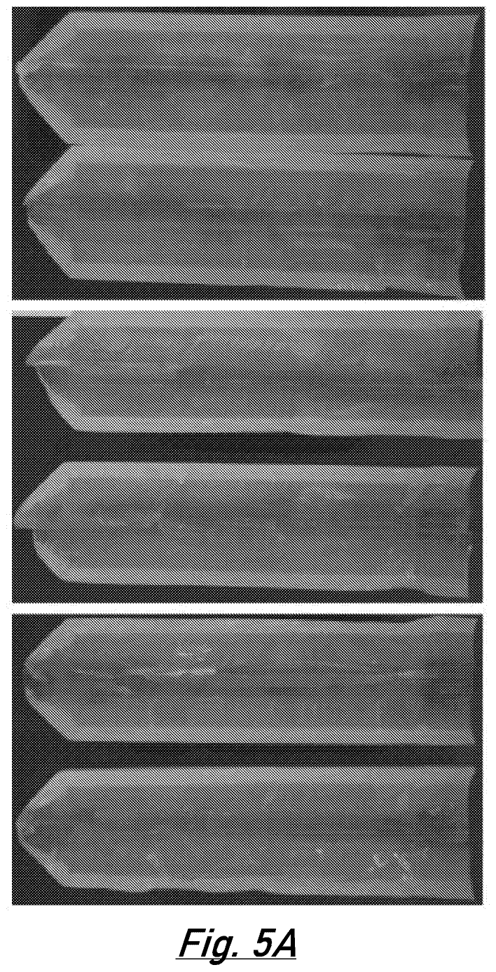

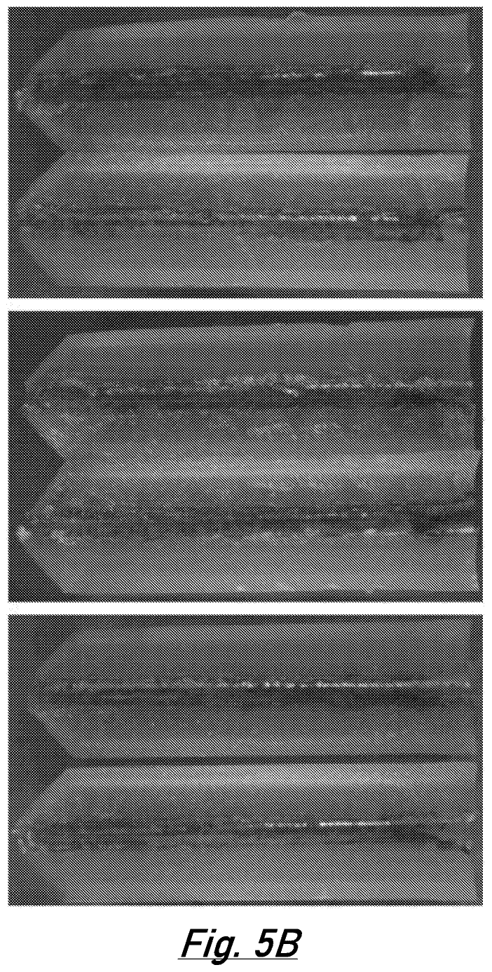

FIG. 5A is a photograph showing a visual displacement image of a Urethral Agar Channel of Example 5A (1000 ppm);

FIG. 5B is a photograph showing a visual displacement image of a Urethral Agar Channel of Example 5B (1000 ppm);

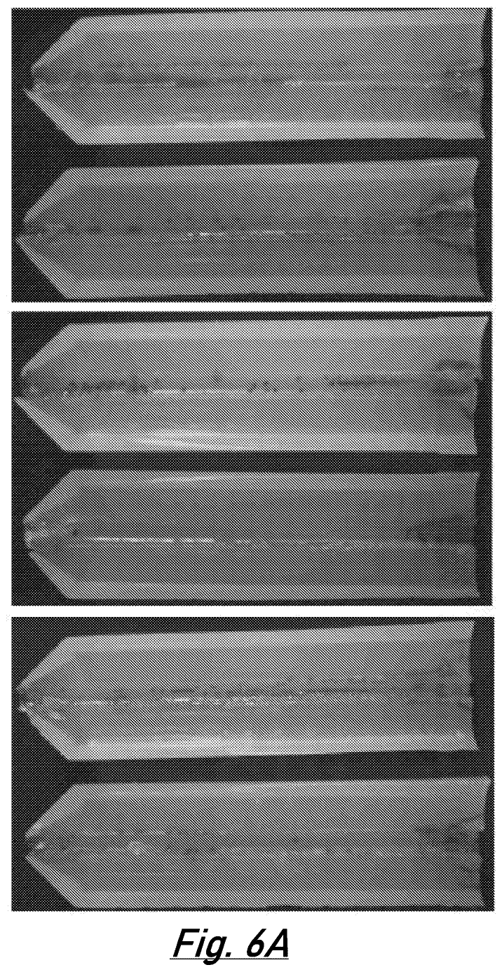

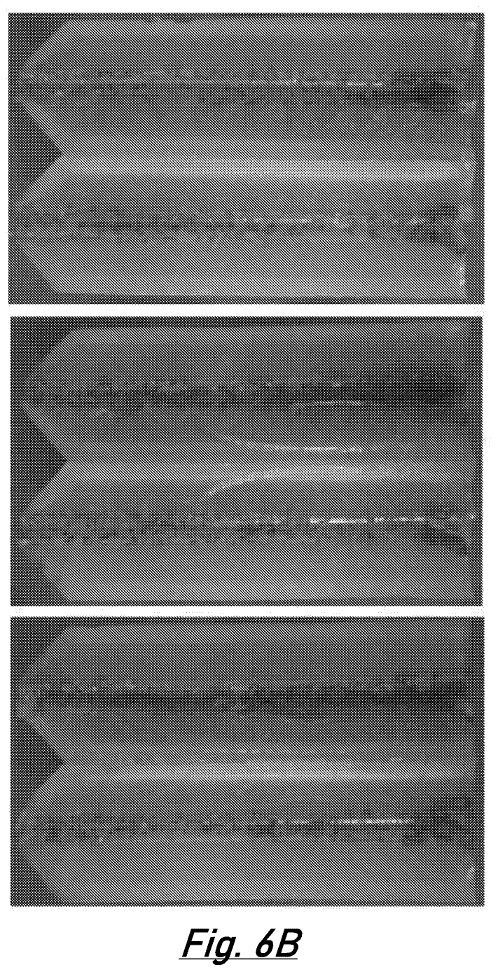

FIG. 6A is a photograph showing a visual displacement image of a Urethral Agar Channel of Example 6 A (2000 ppm);

FIG. 6B is a photograph showing a visual displacement image of a Urethral Agar Channel of Example 6B (2000 ppm);

FIG. 7A is a graph showing the bacterial displacement of Examples 1A. 2A, 3A, 4A, 5A and 6A;

FIG. 7B is a graph showing the bacterial displacement of Examples 1B, 2B, 3B, 4B, 5B and 6B;

FIG. 8 is a chart showing coefficient of friction values for catheters lubricated with a lubricant according to an embodiment of the invention;

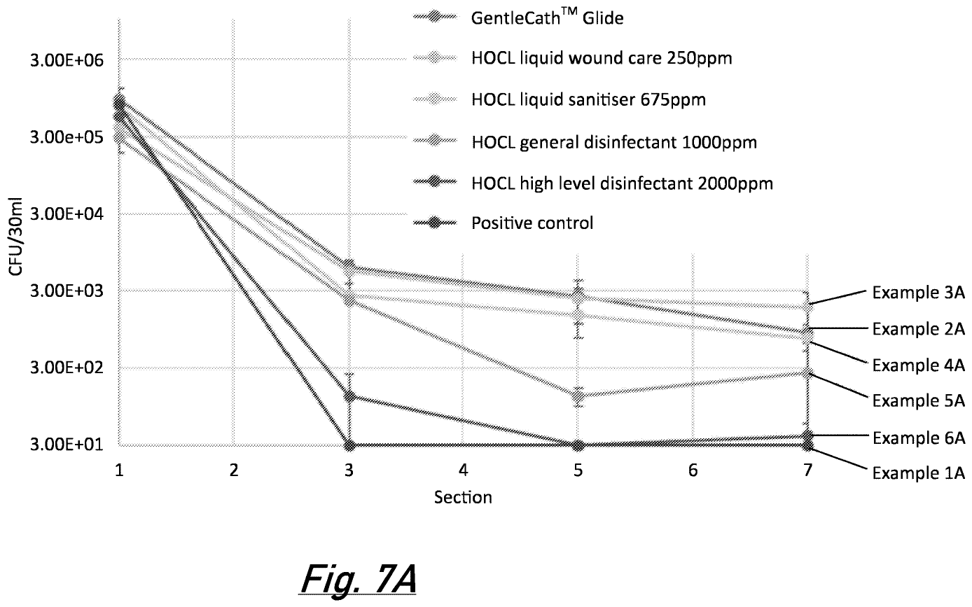

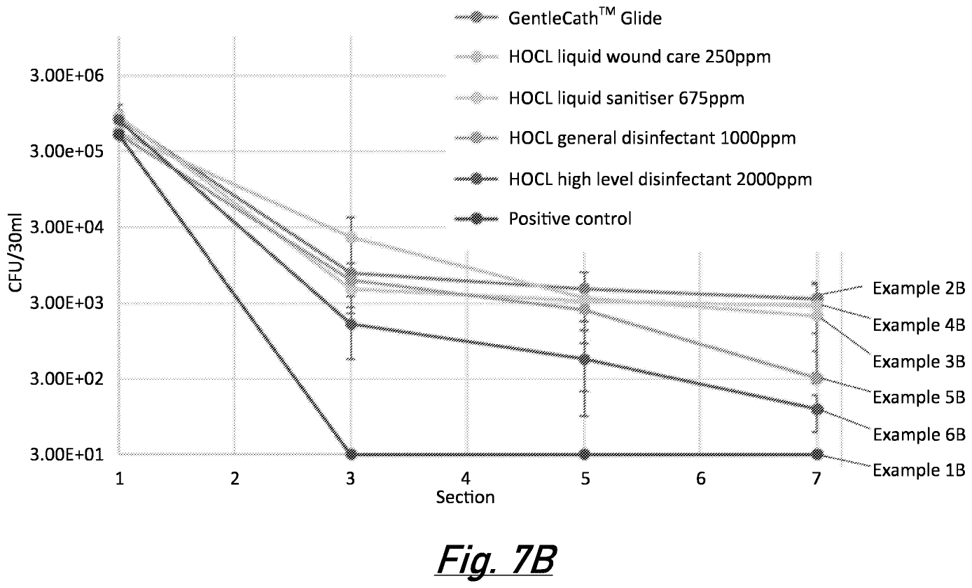

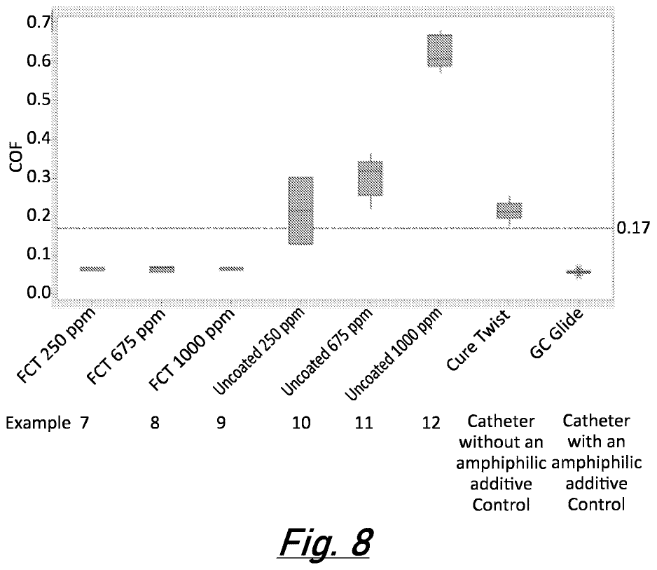

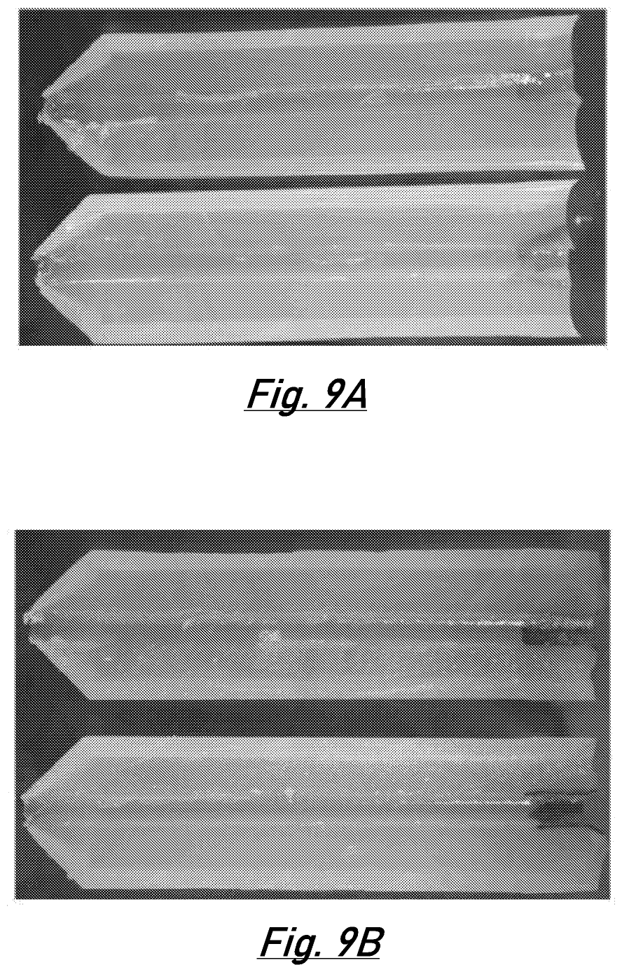

FIG. 9A is a photograph showing a visual displacement image of a Urethral Agar Channel of Example 13 A;

FIG. 9B is a photograph showing a visual displacement image of a Urethral Agar Channel of Example 13B;

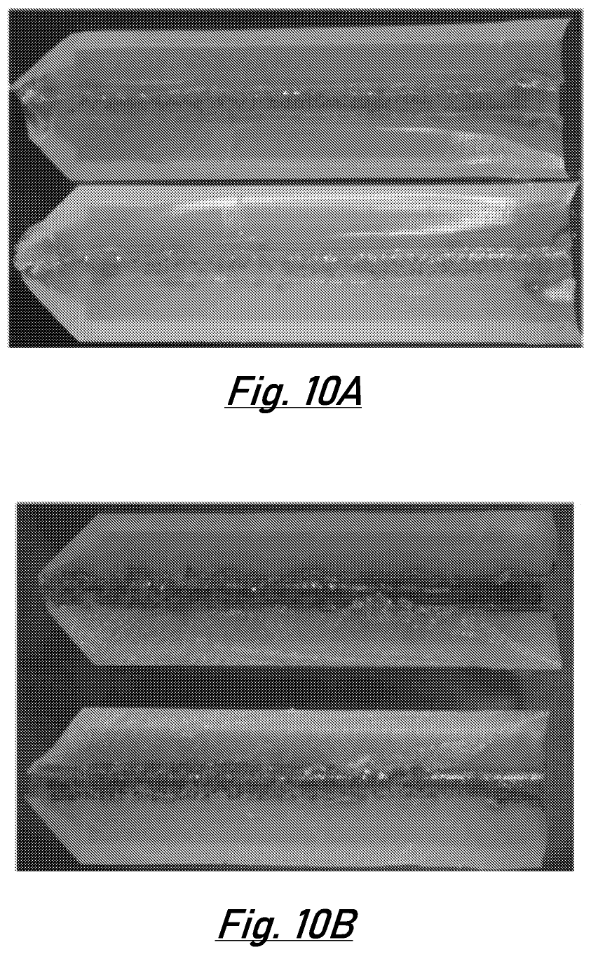

FIG. 10A is a photograph showing a visual displacement image of a Urethral Agar Channel of Example 14A;

FIG. 10B is a photograph showing a visual displacement image of a Urethral Agar Channel of Example 14B;

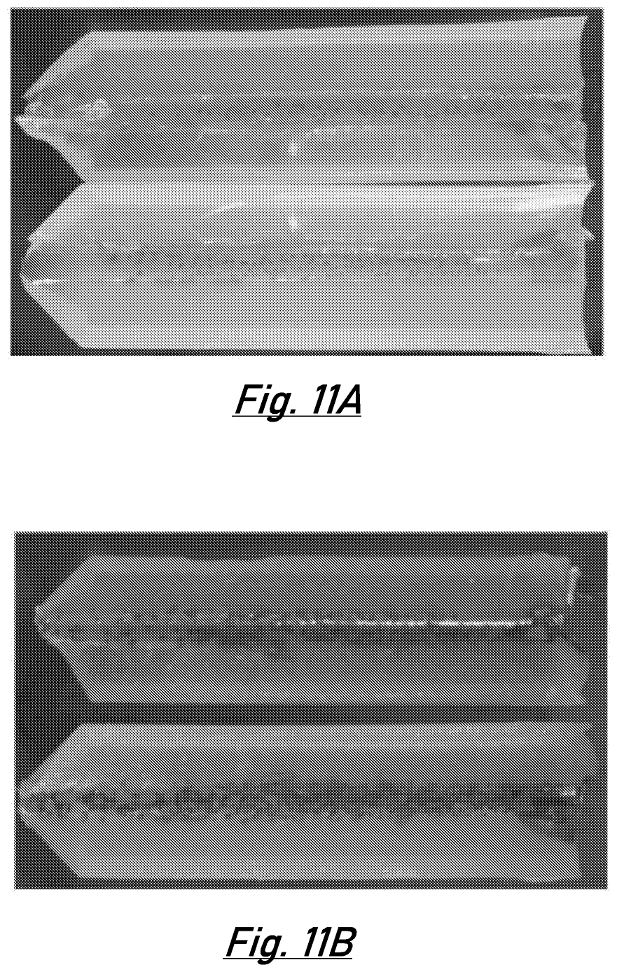

FIG. 11A is a photograph showing a visual displacement image of a Urethral Agar Channel of Example 15 A:

FIG. 11B is a photograph showing a visual displacement image of a Urethral Agar Channel of Example 15B;

FIG. 12A is a photograph showing a visual displacement image of a Urethral Agar Channel of Example 16A;

FIG. 12B is a photograph showing a visual displacement image of a Urethral Agar Channel of Example 16B;

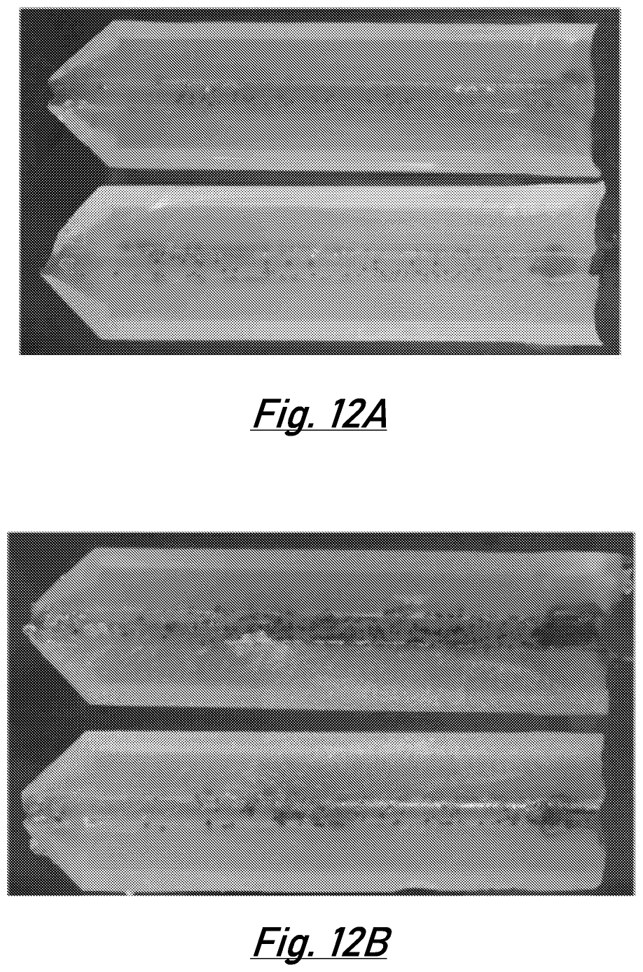

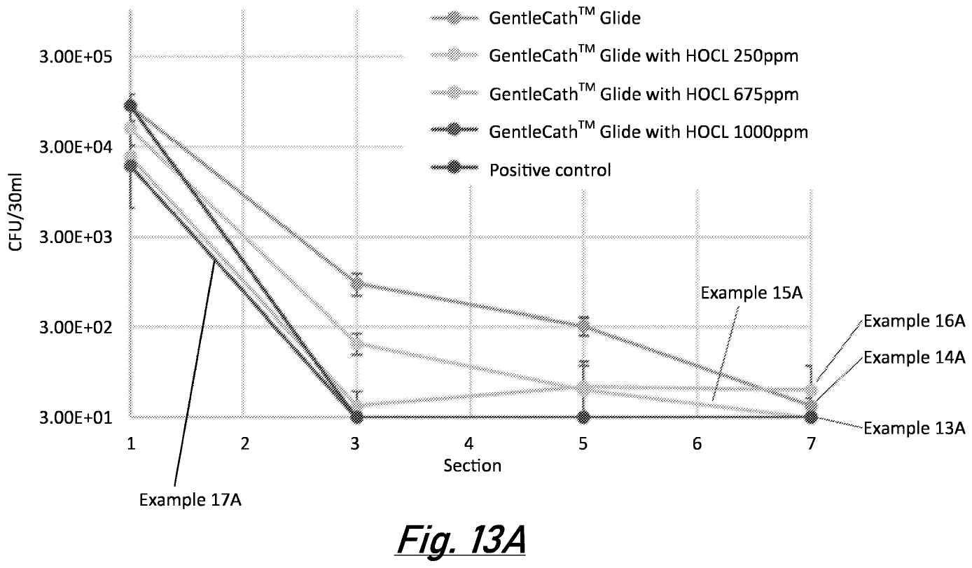

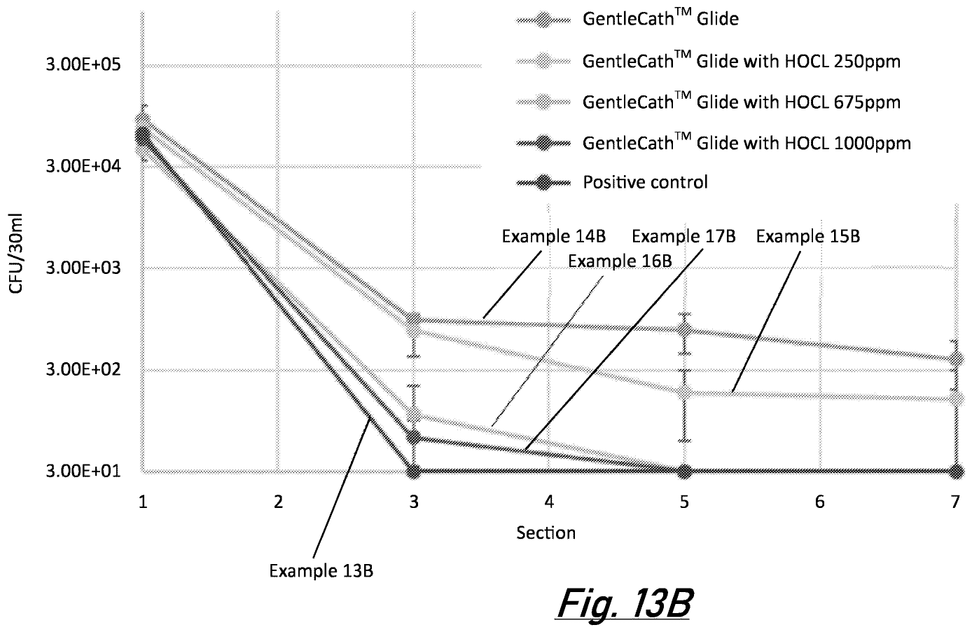

FIG. 13A is a graph showing the bacterial displacement of Examples 13 A, 14A,15A, 16A and 17A;

FIG. 13B is a graph showing the bacterial displacement of Examples 13B, 14B.15B, 16B and 17B;

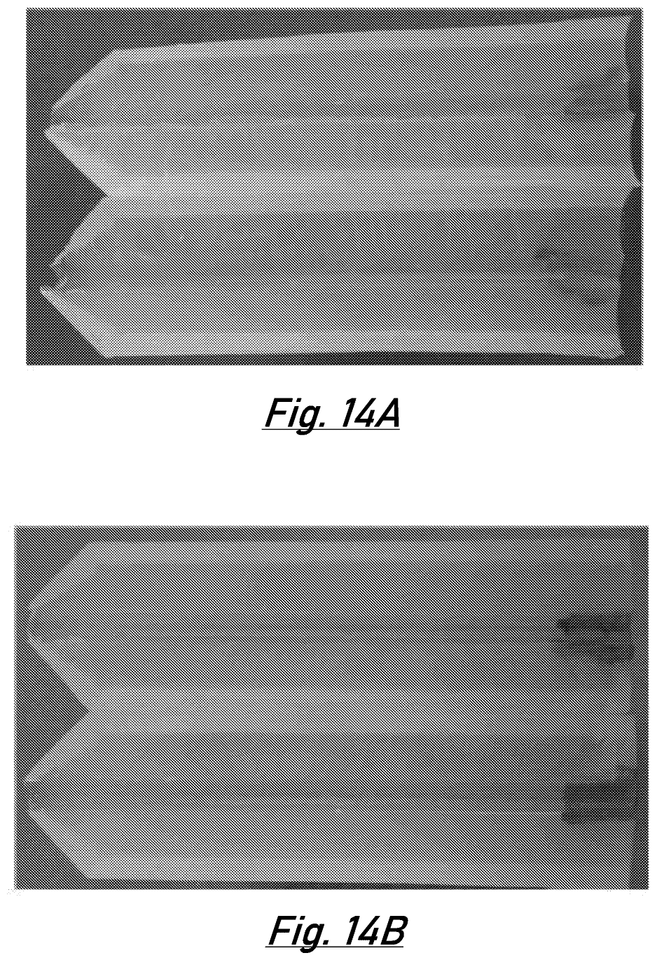

FIG. 14A is a photograph showing a visual displacement image of a Urethral Agar Channel of Example 18A;

FIG. 14B is a photograph showing a visual displacement image of a Urethral Agar Channel of Example 18B;

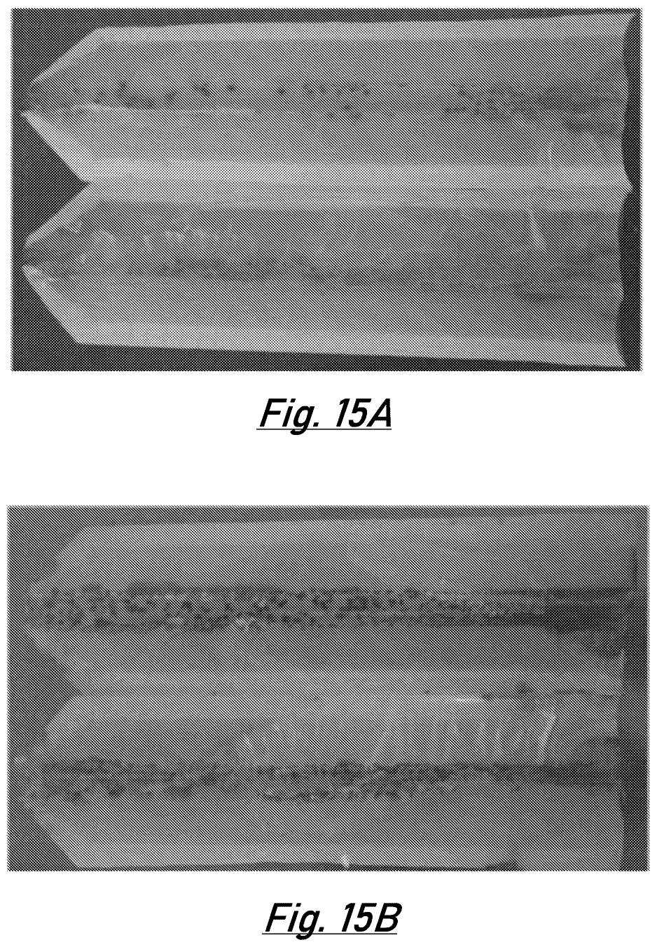

FIG. 15A is a photograph showing a visual displacement image of a Urethral Agar Channel of Example 19A;

FIG. 15B is a photograph showing a visual displacement image of a Urethral Agar Channel of Example 19B;

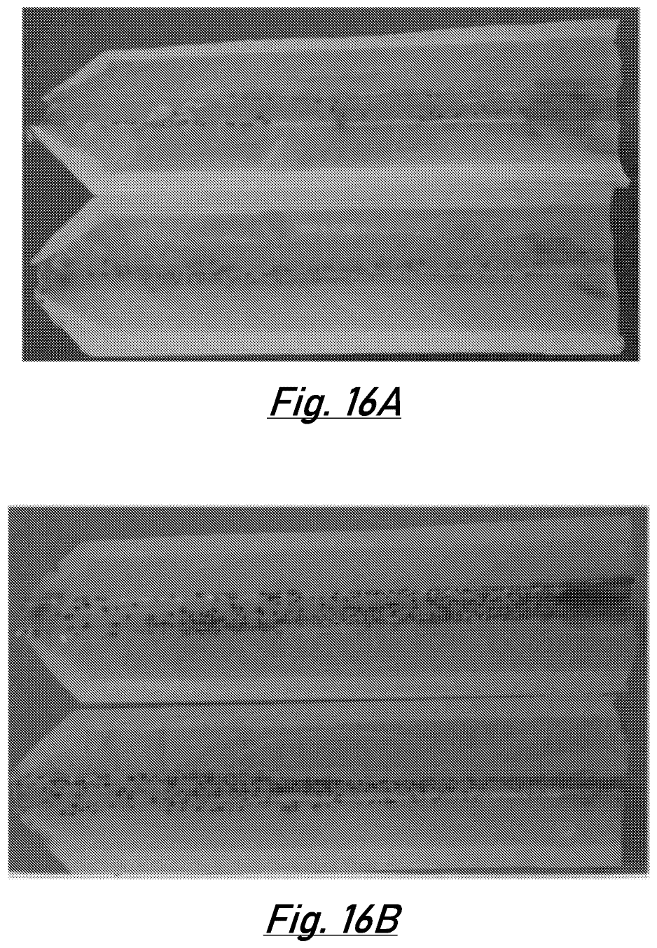

FIG. 16A is a photograph showing a visual displacement image of a Urethral Agar Channel of Example 20A;

FIG. 16B is a photograph showing a visual displacement image of a Urethral Agar Channel of Example 20B;

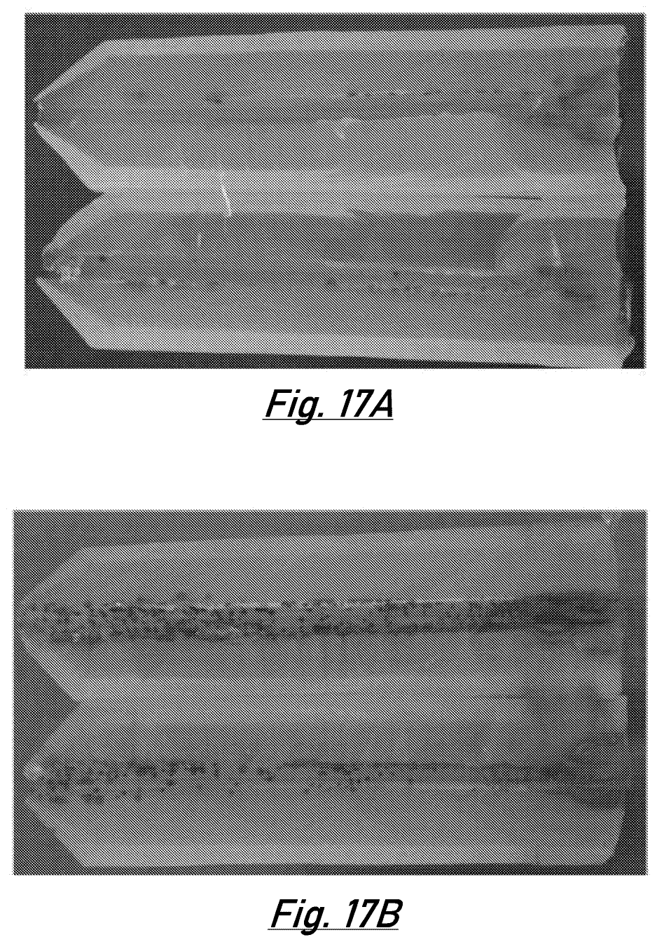

FIG. 17A is a photograph showing a visual displacement image of a Urethral Agar Channel of Example 21 A;

FIG. 17B is a photograph showing a visual displacement image of a Urethral Agar Channel of Example 21 B;

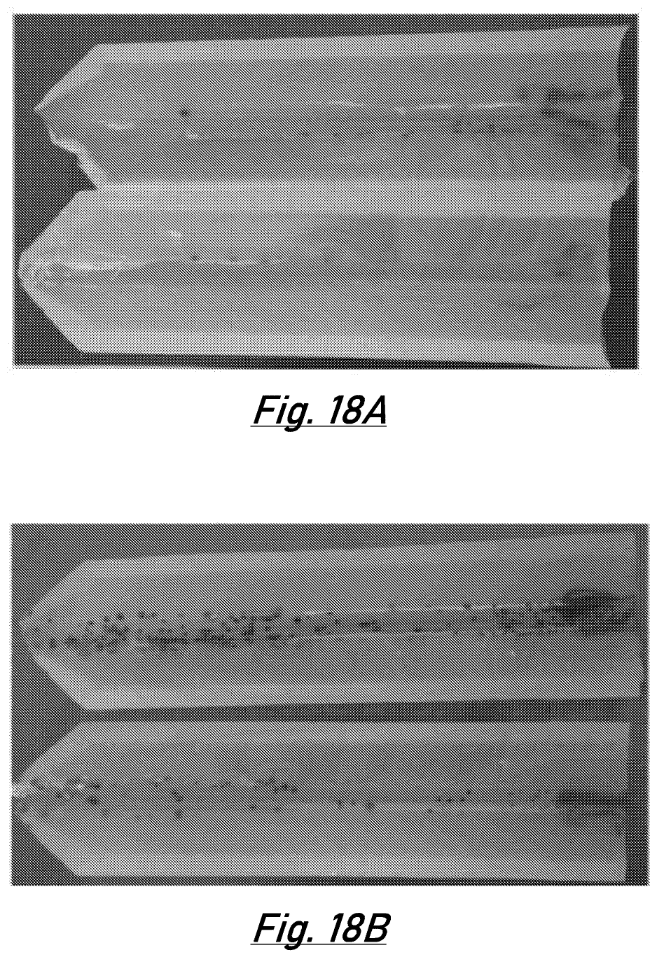

FIG. 18A is a photograph showing a visual displacement image of a Urethral Agar Channel of Example 22A;

FIG. 18B is a photograph showing a visual displacement image of a Urethral Agar Channel of Example 22B;

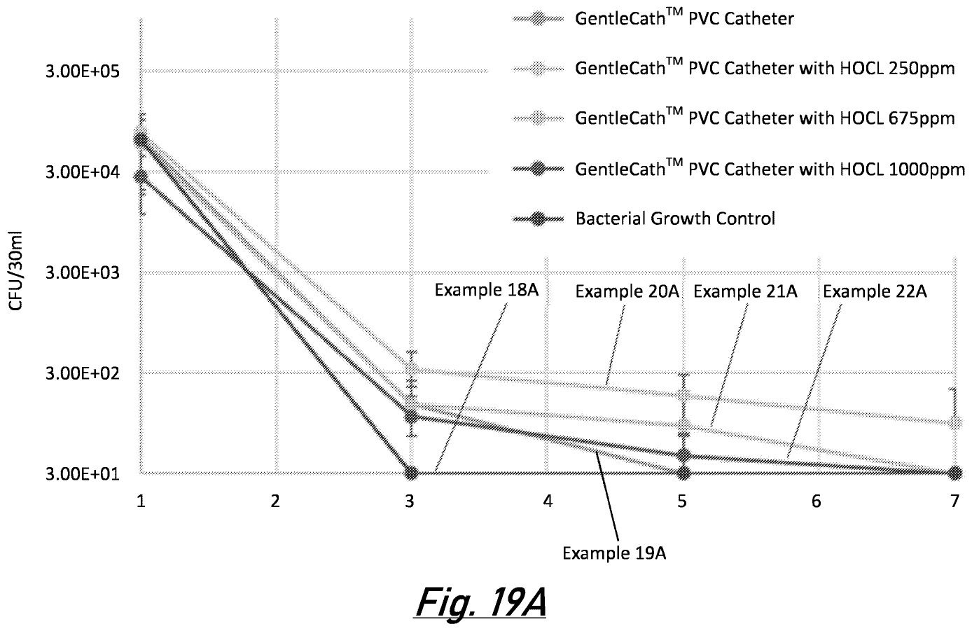

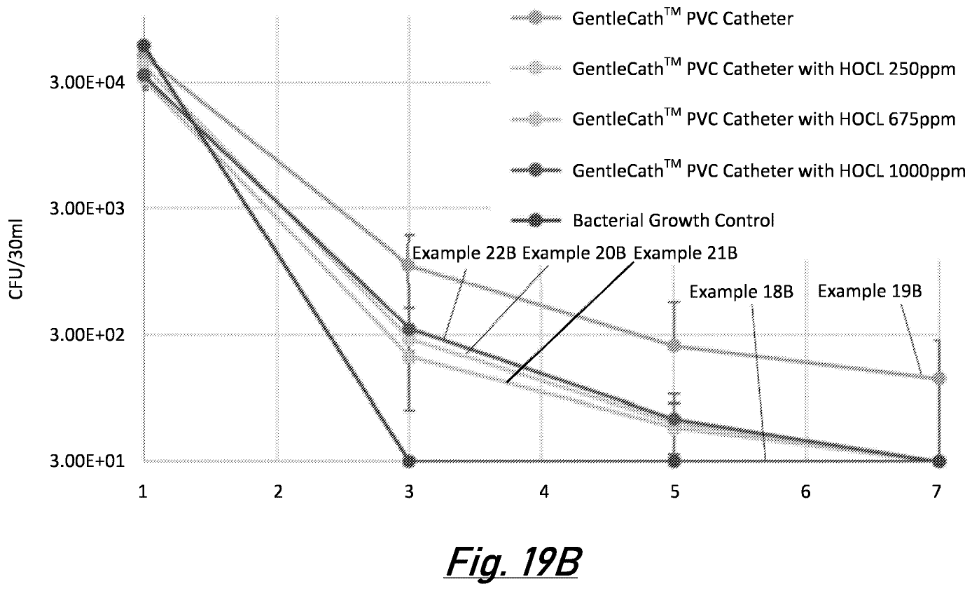

FIG. 19A is a graph showing the bacterial displacement of Examples 18 A, 19A, 20A, 21A and 22A;

FIG. 19B is a graph showing the bacterial displacement of Examples 18B, 19B, 20B, 21B and 22B;

FIG. 20 is a photograph showing a visual displacement image of a Urethral Agar Channel of Example 23;

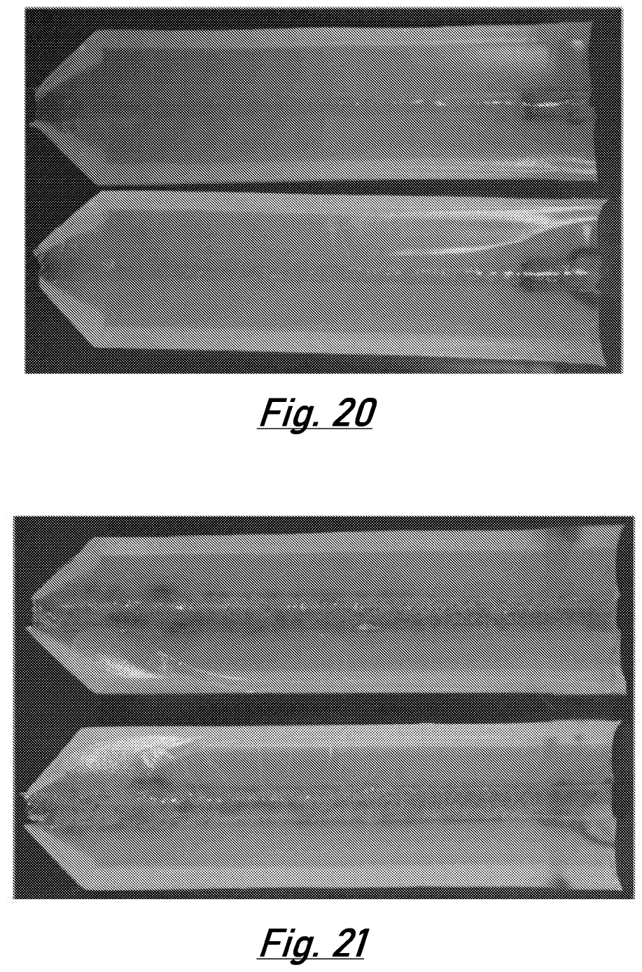

FIG. 21 is a photograph showing a visual displacement image of a Urethral Agar Channel of Example 24;

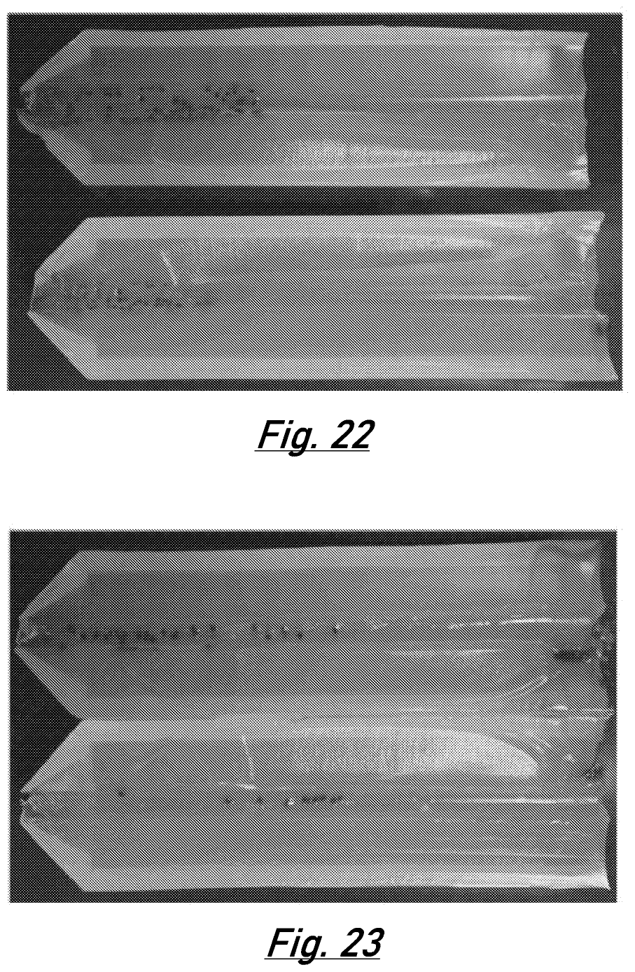

FIG. 22 is a photograph showing a visual displacement image of a Urethral Agar Channel of Example 25;

FIG. 23 is a photograph showing a visual displacement image of a Urethral Agar Channel of Example 26;

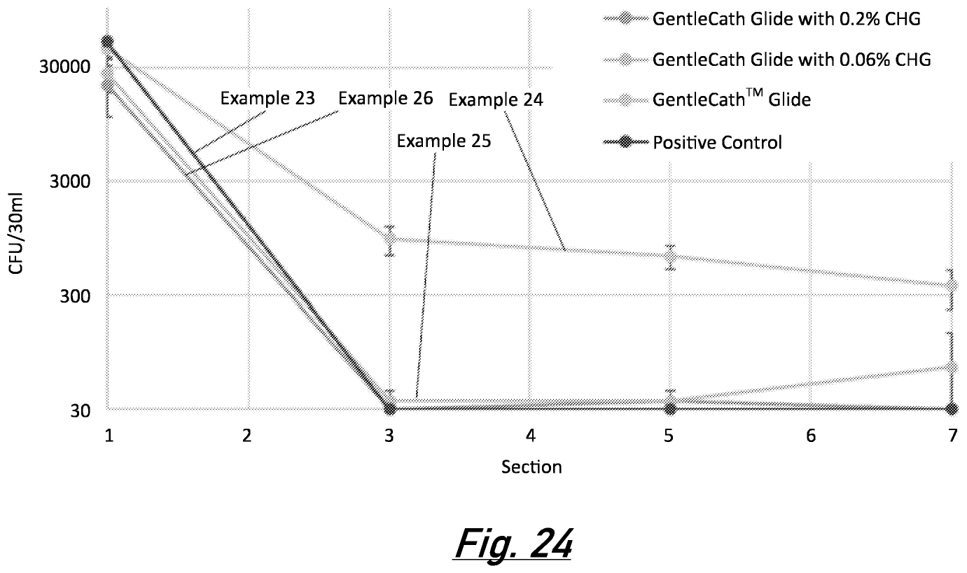

FIG. 24 is a graph showing the bacterial displacement of Examples 23, 24, 25 and 26; and

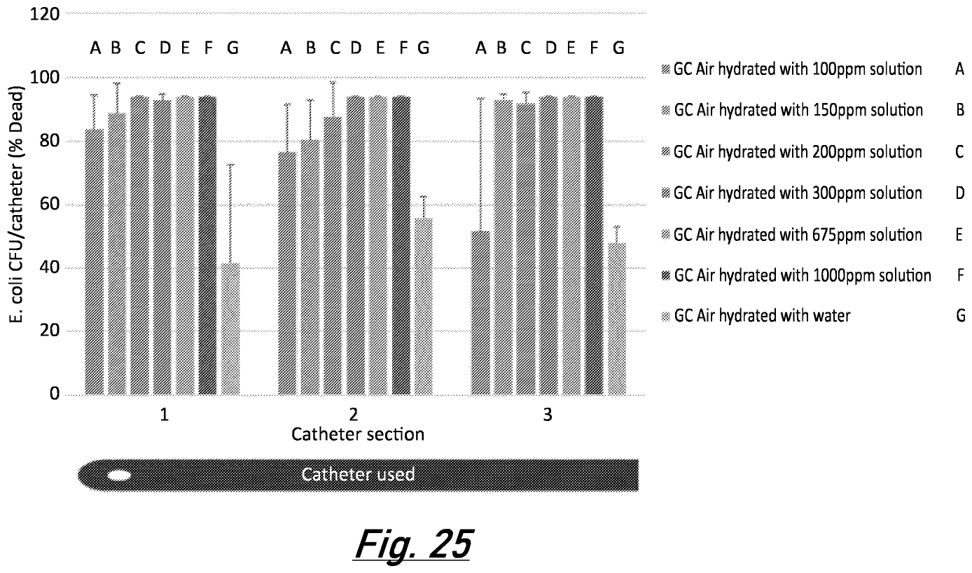

FIG. 25 is a graph showing the percentage of dead E. coli cells relative to the initial number of E. coli cells inoculated onto the surface of each section of catheter.

### Assessment 1—Lubricant Comprising an Antimicrobial Liquid (FIGS. 1-7B)

### Overview

Bacterial transfer from the highly colonised opening of the urethra (distal urethra (meatus)), along the urethra toward the bladder during catheter insertion was tested. To assess the in vitro bacteria displacement potential of the antimicrobial medium of the invention, a microbiology assessment was conducted. The assessment consisted of visual displacement images and Total Viable Counts against inoculation of Escherichia coli NCIMB 14067 and Enterococcus faecalis NCTC 12201 in urethral agar channels representing a user's distal urethra (meatus) and urethra. These organisms were selected as they are commonly identified in catheter associated urinary tract infections.

### Urethral Agar Channel

In each Example, a urethral agar channel was used to measure bacterial transfer along an in vitro urethra channel.

The urethral agar channels were prepared by aseptically dispensing 30 ml of molten Harlequin TBX agar+0.4% AB (a selective agar for Escherichia coli NCIMB 14067) or molten Harlequin VRE chromogenic agar+0.4% AB (a selective agar for Enterococcus faecalis NCTC 12201) into 30 ml universal containers. Sterile 4 mm
stainless steel rods, held in place by sterile stainless steel locating discs, were placed into the universal and removed once set to create a channel down the centre of the agar. The channels were left standing, with their lids removed, within a laminar flow cabinet to dry for 18-24 hours. Before testing, any fluid within the agar channel was removed using a sterile needle and syringe. A sterile cotton swab was then inserted slowly (around 3 seconds) down the length of the channel, rotated anticlockwise twice at the base of the channel and slowly removed.

### Bacteria

Representative colonies from an 18-24 hour challenge culture plate were dispersed in maximum recovery diluent (MRD) to obtain an optical density (OD540 nm) equivalent to approximately IxlO⁸ CFU/ml. 50 pl volumes of this working concentration were transferred into separate bijous for inoculation.

The bacteria tested were Escherichia coli NCIMB 14067 and Enterococcus faecalis NCIMB 12201. The bacteria tested in Examples denoted ‘#A’ was Escherichia coli NCIMB 14067. The bacteria tested in Examples denoted ‘#B’ was Enterococcus faecalis NCIMB 12201.

### Urethral Agar Channel Inoculation

A sterile Hydraflock swab was placed into the 50 pl inoculum of the above working concentration and left to sit for 10 seconds. The inoculated swab was then inserted 1 cm into the insertion entrance (right hand side of each urethral agar channel shown in the Figures) of the urethral agar channel (to represent a user's distal urethra (meatus)), rotated anticlockwise twice and removed. This was performed for all the test and bacterial growth control urethral agar channels. The inoculated urethral agar channels were then left lying horizontally with the lid off and the open, inoculated end facing a flame to dry for 15 minutes. The bacterial growth control image urethral agar channel (see FIGS. 1A and 1B) were incubated at 35±3° C. for 24 hours after inoculation and the bacterial growth control Total Viable Counts (TVCs) urethral agar channel was immediately tested as stated below.

### Catheter

In each Example, the catheter used was a GentleCath™ Glide CH12 male catheter (manufactured by ConvaTec Inc.).

Packaged intermittent urinary catheters were provided. Each packaged intermittent urinary catheter comprised a single use catheter, a packaging surrounding the catheter, and a fluid reservoir holding a lubricant comprising an antimicrobial medium.

The intermittent urinary catheter comprised a hollow polymeric tubular body comprising a thermoplastic elastomer base polymer. The polymeric tubular body of the catheter was compounded with an amphiphilic additive.

The antimicrobial medium was an antimicrobial liquid. The antimicrobial liquid comprised an aqueous chlorine solution. The aqueous chlorine solution comprised at least one chlorine-containing species independently chosen from: hypochlorous acid, at least one hypochlorite salt, chlorine dioxide, at least one chlorhexidine salt, and combinations thereof.

In each Example, the at least one chlorine-containing species was present in an amount of between 100 and 4500 ppm of the overall aqueous content of the fluid reservoir. In each Example, the total concentration of the at least one chlorine-containing species was from 0.05 to 0.5% wt./vol of the overall aqueous content of the fluid reservoir.

In each Example, the catheter had a length of approximately 8 cm.

In the Examples where a lubricant was applied to the catheter, the lubricant was applied as per the IFU, i.e., the catheter was removed from its packaging, cut to approximately 8 cm in length, hydrated with lubricant and immediately inserted into the urethral agar channel.

### Urethral Agar Channel Processing Following Testing for Visual Displacement Images

The catheter is slowly inserted (around 3 seconds) and left in place for 2 minutes to represent urination. Following insertion and removal of the catheter and 15 minutes drying time urethral agar channels were incubated at 35+3° C. for 24 hours. Following incubation, the urethral agar channels were aseptically tipped from the container onto a sterile surface and under aseptic conditions the urethral agar channels were split in half lengthways.

The two halves of the urethral agar channels were then separated so the inside of the channels were visible and growth of the bacteria was therefore also visible. Images were taken to observe the bacteria's displacement through the urethral agar channels.

### Urethral Agar Channel Processing Following Testing for Total Viable Counts (TVCs)

The catheter is slowly inserted (around 3 seconds) into the urethral agar channel and left in place for 2 minutes to represent urination. Following insertion and removal of the catheter and 15 minutes drying time the urethral agar channels for TVCs were aseptically tipped from the universal and cut into 7× 1 cm sections starting at the insertion entry of the urethral agar channel. The 1st, 3rd, 5 th and 7th cm were stomached on high for 4 minutes in MRD+0.01% Tween80 and plated out onto pre-dried Tryptone Soy Agar (TSA) plates in appropriate dilutions. Plates were incubated at 35±3° C. for 48 hours. The number of bacteria (Colony Forming Unit (CFU)) in each 1 cm section of the urethral agar channel was counted (see FIGS. 7A and 7B). The 1^{st} cm starting from the insertion entrance was section 1 and the 1 cm of channel at the end of the urethral agar channel opposite to the insertion entrance was section 7. The limit of detection was 30 CFU.

A high bacterial count (whether measured by TVC or observed through visual displacement images) at the end of the urethral agar channel opposite to the insertion entrance (i.e., section 7) is indicative of high bacteria transfer from the distal urethra (meatus) to the bladder which could increase the risk of the user developing a UTI.

### Example 1—Positive Control

FIGS. 1A and 1B show visual displacement images for a positive control.

Urethral agar channels were prepared as described above. A sterile Hydraflock swab was placed into a 50 pl inoculum of each working concentration of bacteria, prepared as described above, and left to sit for 10 seconds.

The swab inoculated with the working concentration of Escherichia coli NCIMB 14067 was then inserted 1 cm into the insertion entrance of the urethral agar channel of FIG. 1A, rotated anticlockwise twice and removed.

The swab inoculated with the working concentration of Enterococcus faecalis NCIMB 12201 was then inserted lem into the insertion entrance of the urethral agar channel of FIG. 1B. rotated anticlockwise twice and removed.

The inoculated urethral agar channels were then left lying horizontally with the lid off and the open, inoculated end facing a flame to dry for 15 minutes. The bacterial growth control image urethral agar channels were incubated at 35+3° C. for 24 hours after inoculation and the bacterial growth control Total Viable Counts (TVCs) urethral agar channel was immediately tested as stated below.

The two halves of each urethral agar channel were then separated so the inside of the channels was visible and growth of the respective bacteria was therefore also visible. Images (shown as FIGS. 1A and 1B) were taken to observe the bacteria's displacement through each urethral agar channels.

The visual displacement images shown in FIGS. 1A and 1B, and the TVC data shown in FIGS. 7A and 7B, show that each bacteria tested remained in the first lem section of the respective urethra agar channel during incubation. This shows that there were no alternate factors causing migration of the bacteria along the channel.

### Example 2—Catheter Hydrated as Per the IFU

### Example 2A

Urethral agar channels were prepared as described above and inoculated with Escherichia coli NCIMB 14067 (FIG. 2A) in the same manner as described in respect of Example 1.

### Example 2B

Urethral agar channels were prepared as described above and inoculated with Enterococcus faccalis NCIMB 12201 (FIG. 2B) in the same manner as described in respect of Example 1.

### Examples 2A and 2B

A sample size of n=3 was applied to each sample set. Combined TVC data was generated for each 1 cm section of each channel.

Two catheters were prepared as described above. Each catheter was a GentleCath™ Glide (manufactured by ConvaTec Inc.) intermittent urinary catheter. Each catheter was removed from its packaging, hydrated with a water-based lubricant as per the IFU, and tested immediately.

A catheter was slowly inserted (around 3 seconds) into each channel and left in place for 2 minutes to represent urination.

FIGS. 2A and 2B, and 7A and 7B, show that displacement of E. coli NCIMB 14067 and E. faecalis NCIMB 12201 from section 1 to each of sections 2-7 was observed along the entire urethral agar channel.

As such, Example 2 demonstrated that insertion of a catheter readily displaces bacteria from the distal urethra (meatus) and along the urethra.

### Example 3—Catheter Hydrated with Antimicrobial Medium Comprising 0.025% wt./vol. Hypochlorous Acid

### Example 3A

Urethral agar channels were prepared as described above and inoculated with Escherichia coli NCIMB 14067 (FIG. 3A) in the same manner as described in respect of Example 1.

### Example 3B

Urethral agar channels were prepared as described above and inoculated with Enterococcus faecalis NCIMB 12201 (FIG. 3B) in the same manner as described in respect of Example 1.

### Examples 3A and 3B

A sample size of n=3 was applied to each sample set. Combined TVC data was generated for each 1 cm section of each channel.

Two catheters were prepared as described above. Each catheter was a GentleCath™ Glide (manufactured by ConvaTec Inc.) intermittent urinary catheter. Each catheter was removed from its packaging, hydrated with 5 ml of lubricant, as per the IFU, and tested immediately. The lubricant comprised an antimicrobial liquid comprising hypochlorous acid at a concentration of 0.025% wt./vol. (250 ppm).

As shown in FIGS. 3A and 3B, visual displacement of E. coli NCIMB 14067 and E. faecalis NCIMB 12201, respectively, was observed along the entire length of the urethral agar channel.

Example 3B of the invention displayed improved bacterial displacement performance compared to Example 2B, whereby fewer E. faecalis NCIMB 12201 bacteria were displaced along the urethral agar channel when using the catheter of Example 3B, therefore, notably, demonstrating the sterilisation potential of the invention (see FIG. 7B).

### Example 4—Catheter Hydrated with Antimicrobial Medium Comprising 0.0675% wt./vol. Hypochlorous Acid

### Example 4A

Urethral agar channels were prepared as described above and inoculated with Escherichia coli NCIMB 14067 (FIG. 4A) in the same manner as described in respect of Example 1.

### Example 4B

Urethral agar channels were prepared as described above and inoculated with Enterococcus faecalis NCIMB 12201 (FIG. 4B) in the same manner as described in respect of Example 1.

### Examples 4A and 4B

A sample size of n=3 was applied to each sample set. Combined TVC data was generated for each 1 cm section of each channel.

Two catheters were prepared as described above. Each catheter was a GentleCath™ Glide (manufactured by ConvaTec Inc.) intermittent urinary catheter. Each catheter was removed from its packaging, hydrated with 5 ml of lubricant, as per the IFU, and tested immediately.

The lubricant comprised an antimicrobial liquid comprising hypochlorous acid at a concentration of 0.0675% wt./vol. (675 ppm).

It is observed from the visual displacement images shown in FIGS. 4A and 4B that Examples 4A and 4B each show improved bacterial displacement performance compared to Example 2B which used a catheter hydrated with a water-based lubricant and was absent any chlorine-containing species.

This observation is supported by the data provided in FIGS. 7A and 7B which each show that fewer bacteria were displaced from section 1 along the channel toward section 7 when using the catheters of Examples 4A and 4B compared to the catheters of Examples 2 A and 2B.

FIG. 7A shows an improved Escherichia coli NCIMB 14067 displacement performance of Example 4A compared to Example 3A which used a lubricant comprising hypochlorous acid at a concentration of 0.025% wt./vol. (250 ppm).

It is observed in FIG. 7B that Examples 4B and 3B performed similarly in respect of Enterococcus faccalis NCIMB 12201 displacement performance, displacing a similar quantity of bacteria along the urethral agar channel.

These results demonstrate that the catheter of the invention displays improved bacterial displacement performance.

### Example 5—Catheter Hydrated with Antimicrobial Medium Comprising 0.1% wt./vol. Hypochlorous Acid

### Example 5A

Urethral agar channels were prepared as described above and inoculated with Escherichia coli NCIMB 14067 (FIG. 5A) in the same manner as described in respect of Example 1.

### Example 5B

Urethral agar channels were prepared as described above and inoculated with Enterococcus faccalis NCIMB 12201 (FIG. 5B) in the same manner as described in respect of Example 1.

### Examples 5A and 5B

A sample size of n=3 was applied to each sample set. Combined TVC data was generated for each 1 cm section of each channel.

Two catheters were prepared as described above. Each catheter was a GentleCath™ Glide (manufactured by ConvaTec Inc.) intermittent urinary catheter. Each catheter was removed from its packaging, hydrated with 5 ml of lubricant as per the IFU, and tested immediately.

The lubricant comprised an antimicrobial liquid comprising hypochlorous acid at a concentration of 0.1% wt./vol. (1000 ppm).

With respect to visual displacement, Examples 5A and 5B each showed improved bacterial displacement performance compared to Examples 2 A and 2B, respectively. These Figures show fewer bacteria displaced along the urethral agar channel, with a greater number of bacteria visible at the insertion entrance of the urethral agar channel compared to the tip.

Notably. Examples 5A and 5B demonstrate significantly greater bacterial displacement performance compared to that of Examples 2A and 2B, and further improved bacteria displacement performance compared to that of Examples 3 A, 3B, 4A and 4B (see FIGS. 7A and 7B).

Thus, the performance of Examples 5A and 5B demonstrate the reduced bacterial displacement provided by the present invention in that the quantity of bacteria in sections 3, 5 and 7 of each urethral agar channel is significantly below the quantities found in the corresponding sections of Examples 3 A, 4A, 3B and 4B. In particular, it is observed that bacterial displacement performance is significantly improved when the invention comprises an antimicrobial medium comprising hypochlorous acid at a concentration of at least 0.1% wt./vol.

‘11

### Example 6—Catheter Hydrated with Antimicrobial Medium Comprising 0.2% wt./vol. Hypochlorous Acid

### Example 6A

Urethral agar channels were prepared as described above and inoculated with Escherichia coli NCIMB 14067 (FIG. 6A) in the same manner as described in respect of Example 1.

### Example 6B

Urethral agar channels were prepared as described above and inoculated with Enterococcus faecalis NCIMB 12201 (FIG. 6B) in the same manner as described in respect of Example 1.

### Examples 6A and 6B

A sample size of n=3 was applied to each sample set. Combined TVC data was generated for each 1 cm section of each channel.

Two catheters were prepared as described above. Each catheter was a GentleCath™ Glide (manufactured by ConvaTec Inc.) intermittent urinary catheter. Each catheter was removed from its packaging, hydrated with 5 ml of lubricant as per the IFU, and tested immediately.

The lubricant comprised an antimicrobial liquid comprising hypochlorous acid at a concentration of 0.2% wt./vol. (2000 ppm).

As shown in FIGS. 6A and 6B, visual bacterial displacement of Examples 6A and 6B outperformed Examples 2-5 in respect of both Escherichia coli NCIMB 14067 and Enterococcus faecalis NCIMB 12201. In particular, the invention as demonstrated by Example 6A (see FIG. 6A) showed a significant reduction in bacterial displacement along the urethral agar channel compared to all other Examples.

Correspondingly, the TVC of Examples 6A and 6B, as shown in FIGS. 7A and 7B, demonstrates considerably improved bacterial displacement performance of the catheter of the invention compared to the catheters of Examples 2A and 2B. It is demonstrated that Examples 6A and 6B of the invention show the least bacterial displacement along the urethral agar channels compared to Examples 2-5.

Notably, the TVC of section 7 in respect of Examples 6A and 6B reach undetectable levels for E. coli NCIMB 14067 and near-undetectable levels for E. faecalis NCIMB 12201, respectively. Surprisingly, the TVC of Example 6A at section 7 is similar to that of the positive control (Example 1A). It is also observed that the invention as demonstrated by Example 6A is particularly effective against E. coli NCIMB 14067 which is shown to be more susceptible than E. faecalis NCIMB 12201.

### Conclusions

As such, the results demonstrate excellent antimicrobial activity and sterilisation potential of the packaged intermittent urinary catheter of the invention. It is clear that the invention significantly reduces the quantity of bacteria displaced along the urethra when in use. As such, the invention provides for highly effective and improved bacteria displacement performance which, in turn, will reduce the risk of a user developing a UTI.

### Assessment 2—Lubricity Assessment of Antimicrobial Medium (FIG. 8)

### Overview

A lubricity assessment to test the in vitro lubricity of a lubricant of the invention was carried out. The assessment consisted of generating coefficient of friction (COF) values in respect of catheters with an amphiphilic additive and catheters without an amphiphilic additive lubricated with a lubricant of the invention.

### Catheters

A catheter with an amphi philic additive and a catheter without an amphiphilic additive were used in the assessment.

In each Example using a catheter with an amphiphilic additive, the catheter was a urinary intermittent male catheter with FeelClean™ technology produced under the name GentleCath Glide™ (manufactured by ConvaTec Inc.). The intermittent catheter comprised a hollow polymeric tubular body comprising a thermoplastic elastomer base polymer. The polymeric tubular body of the catheter was compounded with the amphiphilic additive. In each Example using a catheter without an amphiphilic additive, the catheter without an amphiphilic additive was a urinary intermittent female PVC catheter produced under the trade name CureTwist® (manufactured by ConvaTec Inc.). The intermittent urinary catheter comprised a hollow polymeric tubular body which was not compounded with an amphiphilic additive.

Packaged intermittent urinary catheters were provided. Each packaged intermittent urinary catheter comprised a single use catheter with an amphiphilic additive or catheter without an amphiphilic additive, a packaging surrounding the catheter, and a fluid reservoir holding a lubricant comprising an antimicrobial medium.

The antimicrobial medium was present as an antimicrobial gel.

The antimicrobial gel comprised an aqueous chlorine solution. The aqueous chlorine solution comprised at least one chlorine-containing species independently chosen from: hypochlorous acid, at least one hypochlorite salt, chlorine dioxide, at least one chlorhexidine salt, and combinations thereof.

In each Example, the at least one chlorine-containing species was present in an amount of between 100 and 4500 ppm of the overall aqueous content of the fluid reservoir. In each Example, the total concentration of the at least one chlorine-containing species was from 0.05 to 0.5% wt./vol of the overall aqueous content of the fluid reservoir.

In each Example, the catheter had a length of approximately 8 cm.

In the Examples where a lubricant was applied to the catheter, the lubricant was applied as per the IFU.

In each Example, the fluid reservoir held 2 ml of the respective test lubricant.

### Sample Preparation

To apply the lubricant, the catheter was removed from its packaging and the test fluid transferred from the fluid reservoir into the packaging. The catheter was inserted into the packaging and at least partially coated in the test lubricant. The catheter was stored in the test lubricant for one hour prior to testing. Separate packaged intermittent urinary catheters were used for each Example.

### Sample Testing

In each Example, the co-efficient of friction was measured using a Harland FTS6000 Friction Testing System (Harland Medical Systems, Inc. USA) by drawing a test sample between two silicone rubber pads clamped at a programmable force, recording the pull resistance and calculating the co-efficient of friction from the results.

A sample size of n=5 was applied to each sample set, with the exception of Example 4 which had a sample size of n=2. Combined COF data was generated for each concentration of the at least one chlorine-containing species. The COF data is shown in Table 1, below, and FIG. 8.

### Positive Control

A catheter with an amphiphilic additive and a catheter without an amphiphilic additive were prepared as described above. In the positive control for the catheter without an amphiphilic additive, the lubricant used was a gel which did not comprise an antimicrobial medium. In the positive control for the catheter with an amphiphilic additive, the lubricant used was water and did not comprise an antimicrobial medium comprising an aqueous chlorine solution.

Each control sample was then tested as described above.

Each positive control provided a benchmark (COF value of <0.17) for the lubricity of gel lubricated catheter without an amphiphilic additive and water-wetted catheters with an amphiphilic additive.

### Examples 7 and 10—Catheters Hydrated with Antimicrobial Gel Comprising 0.025% wt./vol. Hypochlorous Acid

A catheter with an amphiphilic additive (Example 7) and a catheter without an amphiphilic additive (Example 10) were prepared as described above. Each catheter was lubricated with 2 ml of test lubricant, as described above.

In each Example, the lubricant comprised an antimicrobial gel comprising hypochlorous acid at a concentration of 0.025% wt./vol. (250 ppm).

With respect to COF data, Examples 7 and 10 each showed similar lubricity performance compared to the respective positive control.

Thus, the performance of Examples 7 and 10 demonstrate the lubricity of the lubricant provided by the present invention. In particular, it is observed that an antimicrobial gel comprising 0.025% wt./vol. hypochlorous acid does not adversely affect the lubricity of a catheter with an amphiphilic additive or a catheter without an amphiphilic additive when compared to the respective positive control. It is noted that favourable lubricity is particularly found when the catheter of the invention is a catheter with an amphiphilic additive.

### Examples 8 and 11—Catheter Hydrated with Antimicrobial Gel Comprising 0.0675% wt./vol. Hypochlorous Acid

A catheter with an amphiphilic additive (Example 8) and a catheter without an amphiphilic additive (Example 11) were prepared as described above. Each catheter was lubricated with 2 ml of test lubricant, as described above.

In each Example, the lubricant comprised an antimicrobial gel comprising hypochlorous acid at a concentration of 0.0675% wt./vol. (675 ppm).

With respect to COF data, Example 8 showed similar lubricity performance compared to Example 7 and the positive control for catheters with an amphiphilic additive.

Example 11 showed slightly reduced lubricity compared to Example 10 and to the positive control for catheters without an amphiphilic additive. Reduced variability of COF data was also observed in respect of Example 11 compared to the COF data of Example 10.

Thus, the performance of Examples 8 and 11 demonstrate the lubricity of the lubricant provided by the present invention. In particular, it is observed that a lubricant comprising an antimicrobial gel comprising 0.0675% wt./vol. hypochlorous acid does not adversely affect the lubricity of a catheter with an amphiphilic additive when compared to the positive control. It is also observed that a lubricant comprising an antimicrobial gel comprising 0.0675% wt./vol. hypochlorous acid does not have a significant adverse effect on the lubricity of a catheter without an amphiphilic additive when compared to the positive control.

As with Example 7, it is noted that favourable lubricity is particularly found when the catheter of the invention is a catheter with an amphiphilic additive.

### Examples 9 and 12—Catheter Hydrated with Antimicrobial Gel Comprising 0.1% wt./vol. Hypochlorous Acid

A catheter with an amphiphilic additive (Example 9) and a catheter without an amphiphilic additive (Example 12) were prepared as described above. Each catheter was lubricated with 2 ml of test lubricant, as described above.

In each Example, the lubricant comprised an antimicrobial gel comprising hypochlorous acid at a concentration of 0.1% wt./vol. (1000 ppm).

With respect to COF data, Example 9 showed similar lubricity performance compared to Examples 7 and 8, and to the positive control for catheters with an amphiphilic additive.

Example 12 showed reduced lubricity compared to each other Example and to the positive control for catheters without an amphiphilic additive. However, the lubricity of Example 12 is within the expected range for a gel-lubricated catheter without an amphiphilic additive, which is typically a COF value of less than 1.2.

The performance of Example 9 shows that increasing the concentration of hypochlorous acid in the lubricant to 0.1% wt./vol. has no adverse effect on the lubricity of the lubricant when applied to a catheter with an amphiphilic additive.

TABLE 1

| Catheter | | Example 7 | Example 8 | Example 9 |
|---|---|---|---|---|
| Type | Rep | 250 ppm | 675 ppm | 1000 ppm |
| Catheter | 1 | 0.0708 | 0.0714 | 0.0716 |
| with an | 2 | 0.0594 | 0.0688 | 0.0658 |
| amphiphilic | 3 | 0.0596 | 0.0596 | 0.0690 |
| additive | 4 | 0.0624 | 0.0568 | 0.0634 |
| | 5 | 0.0650 | 0.0714 | 0.0686 |

| | | Example 10 | Example 11 | Example 12 |
|---|---|---|---|---|
| Catheter | 1 | 0.1300 | 0.2894 | 0.6052 |
| without an | 2 | 0.3018 | 0.3158 | 0.5690 |
| amphiphilic | 3 | — | 0.2212 | 0.6552 |
| additive | 4 | — | 0.3178 | 0.6036 |
| | 5 | — | 0.3620 | 0.6758 |

### Conclusion

The results demonstrate that the lubricant of an embodiment of the invention has no significant adverse effect on the lubricity of catheters, in particular catheters with an amphiphilic additive. The results of assessment 1 also show that the lubricant of an embodiment of the invention significantly improves antimicrobial activity to minimise bacteria transfer and, therefore, improve bacteria displacement performance, during catheterisation. Thus, a dual effect of the lubricant of the invention is shown.

### Assessment 3—Urinary Intermittent Catheter with an Amphiphilic Additive Hydrated with a Lubricant Comprising an Antimicrobial Gel (FIGS. 9-13)

### Overview

Bacterial transfer from the highly colonised opening of the urethra (distal urethra (meatus)), along the urethra toward the bladder during catheter insertion was tested. To assess the in vitro bacteria displacement potential of the antimicrobial medium of the invention, a microbiology assessment was conducted. The assessment consisted of visual displacement images and Total Viable Counts against inoculation of Escherichia coli NCIMB 14067 and Enterococcus faecalis NCTC 12201 in urethral agar channels representing a user's distal urethra (meatus) and urethra. These organisms were selected as they are commonly identified in catheter associated urinary tract infections.

### Urethral Agar Channel

In each Example, a urethral agar channel was used to measure bacterial transfer along an in vitro urethra channel.

The urethral agar channels were prepared by aseptically dispensing 30 ml of molten Harlequin TBX agar+0.4% AB (a selective agar for Escherichia coli NCIMB 14067) or molten Harlequin VRE chromogenic agar+1% AB (a selective agar for Enterococcus faecalis NCTC 12201) into 30 ml universal containers. Sterile 4 mm stainless steel rods, held in place by sterile stainless steel locating discs, were placed into the universal and removed once set to create a channel down the centre of the agar. The channels were left standing, with their lids removed, within a laminar flow cabinet to dry for 18-24 hours. Before testing, any fluid within the agar channel was removed using a sterile needle and syringe. A sterile cotton swab was then inserted slowly (around 3 seconds) down the length of the channel, rotated anticlockwise twice at the base of the channel and slowly removed.

### Bacteria

Representative colonies from an 18-24 hour challenge culture plate were dispersed in maximum recovery diluent (MRD) to obtain an optical density (OD540 nm) equivalent to approximately 1×10⁸ CFU/ml. This was then diluted ten-fold to obtain a working concentration of I×10⁷ CFU/ml. 50 pl volumes of this working concentration were transferred into separate bijous for inoculation.

The bacteria tested were Escherichia coli NCIMB 14067 and Enterococcus faecalis NCIMB 12201. The bacteria tested in Examples denoted’ #A′ was Escherichia coli NCIMB 14067. The bacteria tested in Examples denoted ‘#B’ was Enterococcus faecalis NCIMB 12201.

### Urethral Agar Channel Inoculation

A sterile Flocked swab was placed into the 50 pl inoculum of the above working concentration and left to sit for 10 seconds. The inoculated swab was then inserted lem into the insertion entrance (right hand side of each urethral agar channel shown in the Figures) of the urethral agar channel (to represent a user's distal urethra (meatus)), rotated anticlockwise twice and removed. This was performed for all the test and bacterial growth control urethral agar channels. The inoculated urethral agar channels were then left lying horizontally with the lid off and the open, inoculated end facing a flame to dry for 15 minutes. The bacterial growth control image urethral agar channel (see FIGS. 9A and 9B) were incubated at 35+3° C. for 24 hours after inoculation and the bacterial growth control Total Viable Counts (TVCs) urethral agar channel was immediately tested as stated below.

### Catheter

In each Example, the catheter used was a GentleCath™ Glide CH12 male catheter (manufactured by ConvaTec Inc.). The catheter was a urinary intermittent catheter.

Packaged intermittent urinary catheters were provided. Each packaged intermittent urinary catheter comprised a single use catheter, a packaging surrounding the catheter, and a fluid reservoir holding a lubricant comprising an antimicrobial medium.

The intermittent urinary catheter comprised a hollow polymeric tubular body comprising a thermoplastic elastomer base polymer. The polymeric tubular body of the catheter was compounded with an amphiphilic additive. As the base polymer is hydrophobic or generally hydrophobic, the amphiphilic additive will diffuse towards and to an outer surface of the catheter body due to incompatibility of the hydrophilic portion of the amphiphilic additive with the hydrophobic base polymer. The amphiphilic additive allows a hydration layer to be created through temporary hydrogen bonds on the catheter surface with water molecules and any chlorine-based species present when the catheter is wetted, such that the surface remains lubricious for longer periods.

The antimicrobial medium was an antimicrobial gel. The antimicrobial gel comprised an aqueous chlorine solution. The aqueous chlorine solution comprised at least one chlorine-containing species independently chosen from: hypochlorous acid, at least one hypochlorite salt, chlorine dioxide, at least one chlorhexidine salt, and combinations thereof.

In each Example, the at least one chlorine-containing species was present in an amount of between 100 and 4500 ppm of the overall aqueous content of the fluid reservoir. In each Example, the total concentration of the at least one chlorine-containing species was from 0.05 to 0.5% wt./vol of the overall aqueous content of the fluid reservoir.

In each Example, the catheter had a length of approximately 8 cm.

In the Examples where a lubricant was applied to the catheter, the lubricant was applied as per the IFU.

### Urethral Agar Channel Processing Following Testing for Visual Displacement Images

The catheter is slowly inserted (around 3 seconds) and left in place for 2 minutes to represent urination. Following insertion and removal of the catheter and 15 minutes drying time urethral agar channels were incubated at 35+3° C. for 24 hours. Following incubation, the urethral agar channels were aseptically tipped from the container onto a sterile surface and under aseptic conditions the urethral agar channels were split in half lengthways.

The two halves of the urethral agar channels were then separated so the inside of the channels were visible and growth of the bacteria was therefore also visible. Images were taken to observe the bacteria's displacement through the urethral agar channels.

### Urethral Agar Channel Processing Following Testing for Total Viable Counts (TVCs)

The catheter is slowly inserted (around 3 seconds) into the urethral agar channel and left in place for 2 minutes to represent urination. Following insertion and removal of the catheter and 15 minutes drying time the urethral agar channels for TVCs were aseptically tipped from the universal and cut into 7× 1 cm sections starting at the insertion entry of the urethral agar channel. The 1st, 3rd, 5 th and 7th cm were stomached on high for 4 minutes in Dey-Engley Neutralising Broth (DENB) and plated out onto pre-dried Tryptone Soy Agar (TSA) plates in appropriate dilutions. Plates were incubated at 35+3° C. for 48 hours. The number of bacteria (Colony Forming Unit
(CFU)) in each 1 cm section of the urethral agar channel was counted (see FIGS. 13A and 13B). The 1st cm starting from the insertion entrance was section 1 and the 1 cm of channel at the end of the urethral agar channel opposite to the insertion entrance was section 7. The limit of detection was 30 CFU.

A high bacterial count (whether measured by TVC or observed through visual displacement images) at the end of the urethral agar channel opposite to the insertion entrance (i.e., section 7) is indicative of high bacteria transfer from the distal urethra (meatus) to the bladder which could increase the risk of the user developing a UTI.

### Example 13—Positive Control

FIGS. 9A and 9B show visual displacement images for a positive control.

Urethral agar channels were prepared as described above. A sterile Flocked swab was placed into a 50 pl inoculum of each working concentration of bacteria, prepared as described above, and left to sit for 10 seconds.

The swab inoculated with the working concentration of Escherichia coli NCIMB 14067 was then inserted 1 cm into the insertion entrance of the urethral agar channel of FIG. 9A, rotated anticlockwise twice and removed.

The swab inoculated with the working concentration of Enterococcus faecalis NCIMB 12201 was then inserted lem into the insertion entrance of the urethral agar channel of FIG. 9B. rotated anticlockwise twice and removed.

The inoculated urethral agar channels were then left lying horizontally with the lid off and the open, inoculated end facing a flame to dry for 15 minutes. The bacterial growth control image urethral agar channels were incubated at 35+3° C. for 24 hours after inoculation and the bacterial growth control Total Viable Counts (TVCs) urethral agar channel was immediately tested as stated below.

The two halves of each urethral agar channel were then separated so the inside of the channels was visible and growth of the respective bacteria was therefore also visible. Images (shown as FIGS. 9A and 9B) were taken to observe the bacteria's displacement through each urethral agar channels.

The visual displacement images shown in FIGS. 9A and 9B, and the TVC data shown in FIGS. 13A and 13B, show that each bacteria tested remained in the first 1 cm section of the respective urethra agar channel during incubation. This shows that there were no alternate factors causing migration of the bacteria along the channel.

### Example 14—Catheter Hydrated as Per the IFU

### Example 14A

Urethral agar channels were prepared as described above and inoculated with Escherichia coli NCIMB 14067 (FIG. 10A) in the same manner as described in respect of Example 13.

### Example 14B

Urethral agar channels were prepared as described above and inoculated with Enterococcus faccalis NCIMB 12201 (FIG. 10B) in the same manner as described in respect of Example 13.

### Examples 14A and 14B

A sample size of n=l was applied to each sample set. Combined TVC data was generated for each 1 cm section of each channel.

Two catheters were prepared as described above. Each catheter was a GentleCath™ Glide CH 12 male intermittent urinary catheter (manufactured by ConvaTec Inc.) as described above. Each catheter was hydrated with a water-based lubricant as per the IFU, removed from its packaging and tested immediately.

A catheter was slowly inserted (around 3 seconds) into each channel and left in place for 2 minutes to represent urination.

FIGS. 10A and 10B, and 13A and 13B, show that displacement of E. coli NCIMB 14067 and E. faecalis NCIMB 12201 from section 1 to each of sections 2-7 was observed along the entire urethral agar channel.

As such, Example 14 demonstrated that insertion of a catheter readily displaces bacteria from the distal urethra (meatus) and along the urethra.

### Example 15—Catheter Hydrated with Antimicrobial Gel Comprising 0.025% wt./vol. Hypochlorous Acid

### Example 15A

Urethral agar channels were prepared as described above and inoculated with Escherichia coli NCIMB 14067 (FIG. 11A) in the same manner as described in respect of Example 13.

### Example 15B

Urethral agar channels were prepared as described above and inoculated with Enterococcus faecalis NCIMB 12201 (FIG. 11B) in the same manner as described in respect of Example 13.

### Examples 15A and 15B

A sample size of n=I was applied to each sample set. Combined TVC data was generated for each 1 cm section of each channel.

Two catheters were prepared as described above. Each catheter was a GentleCath™ Glide CH 12 male intermittent urinary catheter (manufactured by ConvaTec Inc.) as described above. Each catheter was hydrated with 5 ml of lubricant, removed from its packaging and tested immediately.

The lubricant comprised an antimicrobial gel comprising hypochlorous acid at a concentration of 0.025% wt./vol. (250 ppm).

As shown in FIGS. 11A and 11B, the quantity of bacteria transferred along the channel was reduced compared to FIGS. 10A and 10B (Examples 14A and 14B, respectively). Thus, a positive trend was observed compared to Examples 14A and 14B which did not comprise a lubricant comprising an antimicrobial medium.

Examples 15A and 15B of the invention displayed improved bacterial displacement performance compared to Examples 14 A and 14B, respectively, whereby fewer E. coli NCIMB 14067 and E. faecalis NCIMB 12201 bacteria, respectively, were displaced along the urethral agar channel when using each respective catheter, therefore, notably demonstrating the sterilisation potential of the invention (see FIGS. 13A and 13B).

### Example 16—Catheter Hydrated with Antimicrobial Gel Comprising 0.0675% wt./vol. Hypochlorous Acid

### Example 16A

Urethral agar channels were prepared as described above and inoculated with Escherichia coli NCIMB 14067 (FIG. 12A) in the same manner as described in respect of Example 13.

### Example 16B

Urethral agar channels were prepared as described above and inoculated with Enterococcus faecalis NCIMB 12201 (FIG. 12B) in the same manner as described in respect of Example 13.

### Examples 16A and 16B

A sample size of n=1 was applied to each sample set. Combined TVC data was generated for each 1 cm section of each channel.

Two catheters were prepared as described above. Each catheter was a GentleCath™ Glide CH 12 male intermittent urinary catheter (manufactured by ConvaTec Inc.) as described above. Each catheter was hydrated with 5 ml of lubricant, removed from its packaging and tested immediately.

The lubricant comprised an antimicrobial gel comprising hypochlorous acid at a concentration of 0.0675% wt./vol. (675 ppm).

Example 16A of the invention displayed significantly lower levels of bacteria at sections 3 and 5 of the agar channel, and similar levels of bacteria at section 7, compared to that of Example 14A.

Example 16B of the invention displayed improved bacterial displacement performance compared to Example 15B, whereby fewer E. faecalis NCIMB 12201 bacteria were displaced along the urethral agar channel when using the catheter of Example 16B, therefore, notably, demonstrating the sterilisation potential of the invention (see FIG. 13B).

Notably, the TVC of sections 5 and 7 in respect of Examples 16B reached undetectable levels of E. faecalis NCIMB 12201, similar to that of the positive control (Example 13B). It is also observed that the invention as demonstrated by Example 16B is particularly effective against E. faecalis NCIMB 12201 which is shown to be more susceptible than E. coli NCIMB 14067 when the lubricant comprised an antimicrobial gel comprising hypochlorous acid at a concentration of 0.0675% wt./vol. (675 ppm).

### Example 17—Catheter Hydrated with Antimicrobial Gel Comprising 0.1% wt./vol. Hypochlorous Acid

### Example 17 A

Urethral agar channels were prepared as described above and inoculated with Escherichia coli NCIMB 14067 in the same manner as described in respect of Example 13.

### Example 17B

Urethral agar channels were prepared as described above and inoculated with Enterococcus faecalis NCIMB 12201 in the same manner as described in respect of Example 13.

### Examples 17 A and 17B

A sample size of n=1 was applied to each sample set. Combined TVC data was generated for each 1 cm section of each channel.

Two catheters were prepared as described above. Each catheter was a GentleCath™ Glide CH 12 male intermittent urinary catheter (manufactured by ConvaTec Inc.) as described above. Each catheter was hydrated with 5 ml of lubricant, removed from its packaging and tested immediately.

The lubricant comprised an antimicrobial gel comprising hypochlorous acid at a concentration of 0.1% wt./vol. (1000 ppm).

Notably, Examples 17A and 17B demonstrate significantly greater bacterial displacement performance compared to that of Examples 14A and 14B. Also, Example 17A displays improved bacteria displacement performance compared to that of Examples 15A and 16A (see FIG. 13A). Moreover, Example 17B displays improved bacteria displacement performance compared to that of Examples 15B and 16B (see FIG. 13B)

Thus, the performance of Examples 17A and 17B demonstrate the reduced bacterial displacement provided by the present invention in that the quantity of bacteria in sections 3, 5 and 7 of each urethral agar channel is significantly below, or at least below, the quantities found in the corresponding sections of Examples 15A, 16A, 15B and 16B.

Notably, TVC of sections 3, 5 and 7 in respect of Examples 17A, and sections 5 and 7 in respect of Example 17B, reached undetectable levels of E. coli NCIMB 14067 and E.faecalis NCIMB 12201, respectively.

It is observed that bacterial displacement performance is significantly improved when the invention comprises an antimicrobial gel comprising hypochlorous acid at a concentration of at least 0.1% wt./vol.

Note that visual displacement images for Examples 17A and 17B were not produced.

### Conclusions

As such, the results demonstrate excellent antimicrobial activity and sterilisation potential of a packaged intermittent urinary catheter of the invention. It is clear that the invention significantly reduces the quantity of bacteria displaced along the urethra when in use. As such, the invention provides for highly effective and improved bacteria displacement performance which, in turn, will reduce the risk of a user developing a UTI.

### Assessment 4—PVC Urinary Intermittent Catheter with a Lubricant Comprising an Antimicrobial Gel (FIGS. 14-19)

### Overview

Bacterial transfer from the highly colonised opening of the urethra (distal urethra (meatus)), along the urethra toward the bladder during catheter insertion was tested. To assess the in vitro bacteria displacement potential of the antimicrobial medium of the invention, a microbiology assessment was conducted. The assessment consisted of visual displacement images and Total Viable Counts against inoculation of Escherichia coli NCIMB 14067 and Enterococcus faecalis NCTC 12201 in urethral agar channels representing a user's distal urethra (meatus) and urethra. These organisms were selected as they are commonly identified in catheter associated urinary tract infections.

### Urethral Agar Channel

In each Example, a urethral agar channel was used to measure bacterial transfer along an in vitro urethra channel.

The urethral agar channels were prepared by aseptically dispensing 30 ml of molten Harlequin TBX agar+0.4% AB (a selective agar for Escherichia coli NCIMB 14067) or molten Harlequin VRE chromogenic agar+1% AB (a selective agar for Enterococcus faecalis NCTC 12201) into 30 ml universal containers. Sterile 4 mm stainless steel rods, held in place by sterile stainless steel locating discs, were placed into the universal and removed once set to create a channel down the centre of the agar. The channels were left standing, with their lids removed, within a laminar flow cabinet to dry for 18-24 hours. Before testing, any fluid within the agar channel was removed using a sterile needle and syringe. A sterile cotton swab was then inserted slowly (around 3 seconds) down the length of the channel, rotated anticlockwise twice at the base of the channel and slowly removed.

### Bacteria

Representative colonies from an 18-24 hour challenge culture plate were dispersed in maximum recovery diluent (MRD) to obtain an optical density (OD540 nm) equivalent to approximately I×lO⁸ CFU/ml. This was then diluted ten-fold to obtain a working concentration of I×lO⁷ CFU/ml. 50 pl volumes of this working concentration were transferred into separate bijous for inoculation.

The bacteria tested were Escherichia coli NCIMB 14067 and Enterococcus faecalis NCIMB 12201. The bacteria tested in Examples denoted ‘#A’ was Escherichia coli NCIMB 14067. The bacteria tested in Examples denoted ‘#B’ was Enterococcus faecalis NCIMB 12201.

### Urethral Agar Channel Inoculation

A sterile Flocked swab was placed into the 50 pl inoculum of the above working concentration and left to sit for 10 seconds. The inoculated swab was then inserted lem into the insertion entrance (right hand side of each urethral agar channel shown in the Figures) of the urethral agar channel (to represent a user's distal urethra (meatus)), rotated anticlockwise twice and removed. This was performed for all the test and bacterial growth control urethral agar channels. The inoculated urethral agar channels were then left lying horizontally with the lid off and the open, inoculated end facing a flame to dry for 15 minutes. The bacterial growth control image urethral agar channel (see FIGS. 14A and 14B) were incubated at 35+3° C. for 24 hours after inoculation and the bacterial growth control Total Viable Counts (TVCs) urethral agar channel was immediately tested as stated below.

### Catheter

In each Example, the catheter used was a GentleCath™ PVC CH12 male intermittent urinary catheter (manufactured by ConvaTec Inc.).

Packaged intermittent urinary catheters were provided. Each packaged intermittent urinary catheter comprised a single use catheter, a packaging surrounding the catheter, and a fluid reservoir holding a lubricant comprising an antimicrobial medium.

The intermittent urinary catheter comprised a hollow polymeric tubular body comprising a base polymer comprising polyvinyl chloride. The polymeric tubular body was not compounded with an amphiphilic additive.

The antimicrobial medium was an antimicrobial gel. The antimicrobial gel comprised an aqueous chlorine solution. The aqueous chlorine solution comprised at least one chlorine-containing species independently chosen from: hypochlorous acid, at least one hypochlorite salt, chlorine dioxide, at least one chlorhexidine salt, and combinations thereof.

In each Example, the at least one chlorine-containing species was present in an amount of between 100 and 4500 ppm of the overall aqueous content of the fluid reservoir. In each Example, the total concentration of the at least one chlorine-containing species was from 0.05 to 0.5% wt./vol of the overall aqueous content of the fluid reservoir.

In each Example, the catheter had a length of approximately 8 cm.

In the Examples where a lubricant was applied to the catheter, the lubricant was applied as per the IFU.

### Urethral Agar Channel Processing Following Testing for Visual Displacement Images

The catheter is slowly inserted (around 3 seconds) and left in place for 2 minutes to represent urination. Following insertion and removal of the catheter and 15 minutes drying time urethral agar channels were incubated at 35+3° C. for 24 hours. Following incubation, the urethral agar channels were aseptically tipped from the container onto a sterile surface and under aseptic conditions the urethral agar channels were split in half lengthways.

The two halves of the urethral agar channels were then separated so the inside of the channels were visible and growth of the bacteria was therefore also visible. Images were taken to observe the bacteria's displacement through the urethral agar channels.

### Urethral Agar Channel Processing Following Testing for Total Viable Counts (TVCs)

The catheter is slowly inserted (around 3 seconds) into the urethral agar channel and left in place for 2 minutes to represent urination. Following insertion and removal of the catheter and 15 minutes drying time the urethral agar channels for TVCs were aseptically tipped from the universal and cut into 7× 1 cm sections starting at the insertion entry of the urethral agar channel. The 1st, 3rd, 5 th and 7th cm were stomached on high for 4 minutes in DENB and plated out onto pre-dried Tryptone Soy Agar (TSA) plates in appropriate dilutions. Plates were incubated at 35+3° C. for 48 hours. The number of bacteria (Colony Forming Unit (CFU)) in each lem section of the urethral agar channel was counted (see FIGS. 19A and 19B). The 1st cm starting from the insertion entrance was section 1 and the 1 cm of channel at the end of the urethral agar channel opposite to the insertion entrance was section 7. The limit of detection was 30 CFU.

A high bacterial count (whether measured by TVC or observed through visual displacement images) at the end of the urethral agar channel opposite to the insertion entrance (i.e., section 7) is indicative of high bacteria transfer from the distal urethra (meatus) to the bladder which could increase the risk of the user developing a UTI.

### Example 18—Positive Control

FIGS. 14A and 14B show visual displacement images for a positive control.

Urethral agar channels were prepared as described above. A sterile Flocked swab was placed into a 50 pl inoculum of each working concentration of bacteria, prepared as described above, and left to sit for 10 seconds.

The swab inoculated with the working concentration of Escherichia coli NCIMB 14067 was then inserted 1 cm into the insertion entrance of the urethral agar channel of FIG. 14A, rotated anticlockwise twice and removed.

The swab inoculated with the working concentration of Enterococcus faecalis NCIMB 12201 was then inserted 1 cm into the insertion entrance of the urethral agar channel of FIG. 14B, rotated anticlockwise twice and removed.

The inoculated urethral agar channels were then left lying horizontally with the lid off and the open, inoculated end facing a flame to dry for 15 minutes. The bacterial growth control image urethral agar channels were incubated at 35+3° C. for 24 hours after inoculation and the bacterial growth control Total Viable Counts (TVCs) urethral agar channel was immediately tested as stated below.

The two halves of each urethral agar channel were then separated so the inside of the channels were visible and growth of the respective bacteria was therefore also visible. Images (shown as FIGS. 14A and 14B) were taken to observe the bacteria's displacement through each urethral agar channels.

The visual displacement images shown in FIGS. 14A and 14B, and the TVC data shown in FIGS. 19A and 19B, show that each bacteria tested remained in the first 1 cm section of the respective urethra agar channel during incubation. This shows that there were no alternate factors causing migration of the bacteria along the channel.

### Example 19—Catheter Hydrated as Per the IFU

### Example 19A

Urethral agar channels were prepared as described above and inoculated with Escherichia coli NCIMB 14067 (FIG. 15A) in the same manner as described in respect of Example 18.

### Example 19B

Urethral agar channels were prepared as described above and inoculated with Enterococcus faecalis NCIMB 12201 (FIG. 15B) in the same manner as described in respect of Example 18.

### Examples 19A and 19B

A sample size of n=l was applied to each sample set. Combined TVC data was generated for each 1 cm section of each channel.

Two catheters were prepared as described above. Each catheter was a GentleCath™ PVC CH12 male intermittent urinary catheter (manufactured by ConvaTec Inc.). Each catheter was removed from its packaging, hydrated with a waterbased lubricant as per the IFU, and tested immediately.

A catheter was slowly inserted (around 3 seconds) into each channel and left in place for 2 minutes to represent urination.

FIGS. 15A and 15B, and 19A and 19B, show that displacement of E. coli NCIMB 14067 and E. faecalis NCIMB 12201 from section 1 to each of sections 2-7 was observed along the entire urethral agar channel.

As such, Example 19 demonstrated that insertion of a catheter readily displaces bacteria from the distal urethra (meatus) and along the urethra.

Notably, Examples 19A and 19B showed comparatively reduced displacement to Examples 14A and 14B, respectively. It is suggested therefore that the PVC catheter of Examples 19A and 19B facilitates bacterial removal to a greater extent than the urinary intermittent catheter (GentleCath™ Glide CH 12 male catheter (manufactured by ConvaTec Inc.)) of Examples 14A and 14B, which comprised an amphiphilic additive.

### Example 20—PVC Catheter Hydrated with Antimicrobial Gel Comprising 0.025% wt./vol. Hypochlorous Acid

### Example 20A

Urethral agar channels were prepared as described above and inoculated with Escherichia coli NCIMB 14067 (FIG. 16A) in the same manner as described in respect of Example 18.

### Example 20B

Urethral agar channels were prepared as described above and inoculated with Enterococcus faecalis NCIMB 12201 (FIG. 16B) in the same manner as described in respect of Example 18.

### Examples 20A and 20B

A sample size of n=1 was applied to each sample set. Combined TVC data was generated for each 1 cm section of each channel.

Two catheters were prepared as described above. Each catheter was a GentleCath™ PVC CH12 male intermittent urinary catheter (manufactured by ConvaTec Inc.). Each catheter was hydrated with 5 ml of lubricant, removed from its packaging and tested immediately.

The lubricant comprised an antimicrobial gel comprising hypochlorous acid at a concentration of 0.025% wt./vol. (250 ppm).

As shown in FIGS. 16A and 16B, visual displacement of E. coli NCIMB 14067 and E. faecalis NCIMB 12201, respectively, was observed along the entire length of the urethral agar channel.

Example 20B of the invention displayed improved bacterial displacement performance compared to Example 19B, whereby fewer E. faecalis NCIMB 12201 bacteria were displaced along the urethral agar channel when using each respective catheter, therefore, notably, demonstrating the sterilisation potential of the invention (see FIG. 19B).

Removal of bacteria was greater in Example 20B compared to Example 20A, suggesting that E.faecalis NCIMB 12201 bacteria are more susceptible to sterilisation at 250 ppm compared to E. coli NCIMB 14067 using a catheter according to the present embodiment of the invention.

### Example 21—PVC Catheter Hydrated with Antimicrobial Gel Comprising 0.0675% wt./vol. Hypochlorous Acid

### Example 21 A

Urethral agar channels were prepared as described above and inoculated with Escherichia coli NCIMB 14067 (FIG. 19A) in the same manner as described in respect of Example 18.

### Example 21B

Urethral agar channels were prepared as described above and inoculated with Enterococcus faccalis NCIMB 12201 (FIG. 19B) in the same manner as described in respect of Example 18.

### Examples 21 A and 21B

A sample size of n=1 was applied to each sample set. Combined TVC data was generated for each 1 cm section of each channel.

Two catheters were prepared as described above. Each catheter was a GentleCath™ PVC CH12 male intermittent urinary catheter (manufactured by ConvaTec Inc.). Each catheter was hydrated with 5 ml of lubricant, removed from its packaging and tested immediately.

The lubricant comprised an antimicrobial gel comprising hypochlorous acid at a concentration of 0.0675% wt./vol. (675 ppm).

Example 21 A of the invention displayed significantly lower levels of bacteria at sections 3, 5 and 7 of the agar channel compared to that of Example 20 A.

Example 2 IB of the invention displayed improved bacterial displacement performance compared to Example 20B, whereby fewer E. faecalis NCIMB 12201 bacteria were displaced along the urethral agar channel when using the catheter of Example 21B, therefore, notably, demonstrating the sterilisation potential of the invention (see FIG. 19B). A significant reduction in bacteria transfer along the urethral agar channel when using the catheter of Example 2 IB compared to that of Example 19B is also shown in FIG. 19B.

Example 22-PVC catheter hydrated with antimicrobial gel comprising 0.1% wt./vol. Hypochlorous Acid

### Example 22A

Urethral agar channels were prepared as described above and inoculated with Escherichia coli NCIMB 14067 (FIG. 18A) in the same manner as described in respect of Example 18.

### Example 22B

Urethral agar channels were prepared as described above and inoculated with Enterococcus faecalis NCIMB 12201 (FIG. 18B) in the same manner as described in respect of Example 18.

### Examples 22A and 22B

A sample size of n=l was applied to each sample set. Combined TVC data was generated for each lem section of each channel.

Two catheters were prepared as described above. Each catheter was a GentleCath™ PVC CH12 male intermittent urinary catheter (manufactured by ConvaTec Inc.). Each catheter was hydrated with 5 ml of lubricant, removed from its packaging and tested immediately.

The lubricant comprised an antimicrobial gel comprising hypochlorous acid at a concentration of 0.1% wt./vol. (1000 ppm).

With respect to visual displacement, Examples 22 A and 22B, as shown in FIGS. 18A and 18B, outperformed Examples 19-21 in respect of both Escherichia coli NCIMB 14067 and Enterococcus faecalis NCIMB 12201.

Notably, FIG. 19A shows that sections 3 and 5 of Example 22A had a lower number of bacteria than corresponding sections of Examples 20 A and 21 A.

Example 22B provided similar bacterial displacement performance to that of Examples 20B and 21B in sections 3 and 5, and each reached undetectable levels of E. faecalis NCIMB 12201 in section 7 of the urethral agar channel, as shown in FIG. 19B. Thus, compared to Example 18B, it is observed that bacterial displacement performance is significantly improved when the lubricant of the invention comprises an antimicrobial gel comprising hypochlorous acid at a concentration of 0.1% wt./vol.

### Conclusions

As such, the results demonstrate that, against E. faecalis NCIMB 12201, a packaged intermittent urinary catheter of the invention provides excellent antimicrobial activity and sterilisation potential. It is clear that the invention significantly reduces the quantity of bacteria displaced along the urethra when in use. As such, the invention provides for highly effective and improved bacteria displacement performance which, in turn, will reduce the risk of a user developing a UTI.

Moreover, against E. coli NCIMB 14067, the invention demonstrates that increasing concentrations of hypochlorous acid provides improved bacterial displacement performance which is clearly shown in the visual displacement images (see FIGS. 15A, 16A, 17A and 18A). This trend is also shown quantitively with the TVC data in respect of Examples 20 A. 21 A and 22A.

### Assessment 5—Urinary Intermittent Catheter with an Amphiphilic Additive Coated with a Lubricant Comprising Chlorhexidine Digluconate (FIGS. 20-23)

### Overview

Bacterial transfer from the highly colonised opening of the urethra (distal urethra (meatus)), along the urethra toward the bladder during catheter insertion was tested. To assess the in vitro bacteria displacement potential of the antimicrobial medium of the invention, a microbiology assessment was conducted. The assessment consisted of visual displacement images and Total Viable Counts against inoculation of Escherichia coli NCIMB 14067 in urethral agar channels representing a user's distal urethra (meatus) and urethra. This organism was selected as it is commonly identified in catheter associated urinary tract infections.

### Urethral Agar Channel

In each Example, a urethral agar channel was used to measure bacterial transfer along an in vitro urethra channel.

The urethral agar channels were prepared by aseptically dispensing 30 ml of molten Harlequin TBX agar+0.4% AB (a selective agar for Escherichia coli NCIMB 14067) into 30 ml universal containers. Sterile 4 mm stainless steel rods, held in place by sterile stainless steel locating discs, were placed into the universal and removed once set to create a channel down the centre of the agar. The channels were the same diameter as a CH12 catheter (see description below). The channels were left standing, with their lids removed, within a laminar flow cabinet to dry for 18-24 hours. Before testing, any fluid within the agar channel was removed using a sterile needle and syringe. A sterile cotton swab was then inserted slowly (around 3 seconds) down the length of the channel, rotated anticlockwise twice at the base of the channel and slowly removed.

### Bacteria

Representative colonies from an 18-24 hour challenge culture plate were dispersed in maximum recovery diluent (MRD) to obtain an optical density (OD540 nm) equivalent to approximately I×lO⁸ CFU/ml. This was then diluted ten-fold to obtain a working concentration of IxlO⁷ CFU/ml. 50 pl volumes of this working concentration were transferred into separate bijous for inoculation.

The bacteria tested was Escherichia coli NCIMB 14067.

### Urethral Agar Channel Inoculation

A sterile Flocked swab was placed into the 50 pl inoculum of the above working concentration and left to sit for 10 seconds. The inoculated swab was then inserted 1 cm into the insertion entrance of the urethral agar channel (to represent a user's distal urethra (meatus)), rotated anticlockwise twice and removed. This was performed for all the test and bacterial growth control urethral agar channels. The inoculated urethral agar channels were then left lying horizontally with the lid off and the open, inoculated end facing a flame to dry for 15 minutes. The bacterial growth control image urethral agar channel was incubated at 35+3° C. for 24 hours after inoculation and the bacterial growth control Total Viable Counts (TVCs) urethral agar channel was immediately tested as stated below.

### Catheter

In each Example, the catheter used was a GentleCath™ Glide CH12 male catheter (manufactured by ConvaTec Inc.). The catheter was a urinary intermittent catheter.

Packaged intermittent urinary catheters were provided. Each packaged intermittent urinary catheter comprised a single use catheter, a packaging surrounding the catheter, and a fluid reservoir holding a lubricant comprising an antimicrobial medium.

The intermittent urinary catheter comprised a hollow polymeric tubular body comprising a thermoplastic elastomer base polymer. The polymeric tubular body of the catheter was compounded with an amphiphilic additive.

The antimicrobial medium was an antimicrobial liquid. The antimicrobial liquid comprised an aqueous chlorine solution. The aqueous chlorine solution comprised at least one chlorine-containing species independently chosen from: hypochlorous acid, at least one hypochlorite salt, chlorine dioxide, at least one chlorhexidine salt, and combinations thereof.

In each Example, the at least one chlorine-containing species was present in an amount of between 100 and 4500 ppm of the overall aqueous content of the fluid reservoir. In each Example, the total concentration of the at least one chlorine-containing species was from 0.05 to 0.5% wt./vol of the overall aqueous content of the fluid reservoir.

In each Example, the catheter had a length of approximately 8 cm.

In the Examples where a lubricant was applied to the catheter, the lubricant was applied as per the IFU.

### Urethral Agar Channel Processing Following Testing for Visual Displacement Images

The catheter is slowly inserted (around 3 seconds) and left in place for 2 minutes to represent urination. Following insertion and removal of the catheter and 15 minutes drying time urethral agar channels were incubated at 35+3° C. for 24 hours. Following incubation, the urethral agar channels were aseptically tipped from the container onto a sterile surface and under aseptic conditions the urethral agar channels were split in half lengthways.

The two halves of the urethral agar channels were then separated so the inside of the channels was visible and growth of the bacteria was therefore also visible. Images were taken to observe the bacteria's displacement through the urethral agar channels.

### Urethral Agar Channel Processing Following Testing for Total Viable Counts (TVCs)

The catheter is slowly inserted (around 3 seconds) into the urethral agar channel and left in place for 2 minutes to represent urination. Following insertion and removal of the catheter and 15 minutes drying time the urethral agar channels for TVCs were aseptically tipped from the universal and cut into 7× 1 cm sections starting at the insertion entry of the urethral agar channel. The 1st, 3rd, 5 th and 7th cm were stomached on high for 4 minutes in DENB and plated out onto pre-dried Tryptone Soy Agar (TSA) plates in appropriate dilutions. Plates were incubated at 35+3° C. for 48 hours. The number of bacteria (Colony Forming Unit (CFU)) in each 1 cm section of the urethral agar channel was counted. The 1st cm starting from the insertion entrance was section 1 and the 1 cm of channel at the end of the urethral agar channel opposite to the insertion entrance was section 7. The limit of detection was 30 CFU.

A high bacterial count (whether measured by TVC or observed through visual displacement images) at the end of the urethral agar channel opposite to the insertion entrance (i.e., section 7) is indicative of high bacteria transfer from the distal urethra (meatus) to the bladder which could increase the risk of the user developing a UTI.

### Example 23—Positive Control

FIG. 20 shows visual displacement images for a positive control.

Urethral agar channels were prepared as described above. A sterile Flocked swab was placed into a 50 pl inoculum of each working concentration of bacteria, prepared as described above, and left to sit for 10 seconds.

The swab inoculated with the working concentration of Escherichia coli NCIMB 14067 was then inserted lem into the insertion entrance of a urethral agar channel, rotated anticlockwise twice and removed.

The inoculated urethral agar channel was then left lying horizontally with the lid off and the open, inoculated end facing a flame to dry for 15 minutes. The bacterial growth control image urethral agar channels were incubated at 35+3° C. for 24 hours after inoculation and the bacterial growth control Total Viable Counts (TVCs) urethral agar channel was immediately tested as stated below.

The two halves of each urethral agar channel were then separated so the inside of the channel was visible and growth of the respective bacteria was therefore also visible. Image (FIG. 20) was taken to observe the bacteria's displacement through each urethral agar channels.

The visual displacement image shown in FIG. 20, and the TVC data shown in FIG. 24, show that the bacteria tested remained in the first 1 cm section of the urethra agar channel during incubation. This shows that there were no alternate factors causing migration of the bacteria along the channel.

### Example 24—Catheter Hydrated as Per the IFU

A urethral agar channel was prepared as described above and inoculated with Escherichia coli NCIMB 14067 (FIG. 21) in the same manner as described in respect of Example 23.

A sample size of n=1 was applied to the sample set. Combined TVC data was generated for each lem section of the channel.

A catheter was prepared as described above. The catheter was a GentleCath™ Glide CH12 male intermittent urinary catheter (manufactured by ConvaTec Inc.) as described above. The catheter was hydrated with a water-based lubricant as per the IFU, removed from its packaging and tested immediately.

A catheter was slowly inserted (around 3 seconds) into each channel and left in place for 2 minutes to represent urination.

FIGS. 21 and 24 show that displacement of E. coli NCIMB 14067 from section 1 to each of sections 2-7 was observed along the entire urethral agar channel.

As such, Example 24 demonstrated that insertion of a catheter readily displaces bacteria from the distal urethra (mcatus) and along the urethra.

### Example 25—Catheter Hydrated with Antimicrobial Liquid Comprising 0.06% wt./vol. Chlorhexidine Digluconate (CHG)

A wrethral agar channel was prepared as described above and inoculated with Escherichia coli NCIMB 14067 (FIG. 22) in the same manner as described in respect of Example 23.

A sample size of n=1 was applied to the sample set. Combined TVC data was generated for each 1 cm section of the channel.

A catheter was prepared as described above. The catheter was a GentleCath™ Glide CH12 male intermittent urinary catheter (manufactured by ConvaTec Inc.) as described above. The catheter was hydrated with 5 ml of lubricant, removed from its packaging and tested immediately.

The lubricant comprised an antimicrobial liquid comprising chlorhexidine digluconate at a concentration of 0.06% wt./vol. (600 ppm).

As shown in FIG. 22, the quantity of bacteria transferred along the channel was reduced compared to FIG. 21 (Example 24). Thus, a positive trend was observed compared to Example 24 which did not comprise a lubricant comprising an antimicrobial medium.

Example 25 of the invention displayed improved bacterial displacement performance compared to Example 24, whereby fewer E. coli NCIMB 14067 bacteria were displaced along the urethral agar channel when using the catheter of Example 25, therefore, notably demonstrating the sterilisation potential of the invention.

Notably, the TVC of sections 5 and 7 in respect of Example 25 reached near undetectable levels of E. coli NCIMB 14067, similar to that of the positive control (Example 23).

### Example 26—Catheter Hydrated with Antimicrobial Liquid Comprising 0.2% wt./vol. Chlorhexidine Digluconate

### Example 26

A urethral agar channel was prepared as described above and inoculated with Escherichia coli NCIMB 14067 (FIG. 23) in the same manner as described in respect of Example 23.

A sample size of n=1 was applied to the sample set. Combined TVC data was generated for each 1 cm section of each channel.

A catheter was prepared as described above. The catheter was a GentleCath™ Glide CH12 male intermittent urinary catheter (manufactured by ConvaTec Inc.) as described above. The catheter was hydrated with 5 ml of lubricant, removed from its packaging and tested immediately.

The lubricant comprised an antimicrobial liquid comprising chlorhexidine digluconate at a concentration of 0.2% wt./vol. (2000 ppm).

Example 26 of the invention displayed significantly lower levels of bacteria at sections 3 and 5 of the agar channel, and similar levels of bacteria at section 7, compared to that of Example 24.

Example 26 of the invention displayed improved bacterial displacement performance compared to Example 25, whereby fewer E. coli NCIMB 14067 bacteria were displaced along the urethral agar channel when using the catheter of Example 26, therefore, notably, demonstrating the sterilisation potential of the invention (see FIG. 24).

Notably, the TVC of sections 5 and 7 in respect of Example 26 reached undetectable levels of E. coli NCIMB 14067, similar to that of the positive control (Example 23).

### Conclusions

As such, the results demonstrate excellent antimicrobial activity and sterilisation potential of a packaged intermittent urinary catheter of the invention. It is clear that the invention significantly reduces the quantity of bacteria displaced along the urethra when in use. Sterilisation can be achieved by as simply as contacting the catheter with the medium, such as by submerging the catheter in the medium. As such, the invention provides for highly effective and improved bacteria displacement performance which, in turn, will reduce the risk of a user developing a UTI.

### Assessment 6—Antimicrobial Activity of Hypochlorous Acid (HOC1)

### Overview

Testing has been performed to evaluate various hypochlorous acid concentrations in various test methods. The testing included challenge organisms being added directly into a solution (assessment 6a), and along the length of a catheter once it has been hydrated with hypochlorous acid solution (Assessment 6b). The assessment further included testing the residual activity of hypochlorous acid to establish how long the hypochlorous acid remains active against the challenge organism (Assessment 6c).

### Assessment 6a—Direct Inoculation Method

### Bacteria

Representative colonies from an 18-24 hour challenge culture plate were dispersed in a 500pl aliquot of maximum recovery diluent (MRD) to obtain an optical density (OD540 nm) equivalent to approximately I×lO⁸ CFU/ml. This was diluted to obtain a working concentration of I×lO⁷ CFU/ml. A quantitative count was performed to confirm the inoculum level.

The bacteria tested were Escherichia coli NCIMB 14067, ESBL Producing Klebsiella pneumoniae NCTC 13465, Enterococcus faecalis NCTC 12201, Proteus mirabilis NCTC 9559 and Pseudomonas aeruginosa NCIMB 8626, which each are commonly associated with causing CAUTI.

Test method

5 pl of the I×lO⁷ CFU/ml challenge suspension was added to a 500 pl aliquot of hypochlorous acid solution (250 ppm) (n=3 per challenge organism, per timepoint) and mixed to form a suspension.

Following 10 seconds, 1 minute and 2 minutes from adding the challenge suspension to the aliquot of hypochlorous acid, samples were taken from the suspension to establish the number of challenge organisms remaining. This was performed by removing 100 pl volumes and transferring to 0.9 ml and 9.9 ml DENB (for 1:10 and 1:100 dilutions respectively). Appropriate serial dilutions were performed and inoculated onto pre-dried Tryptone Soy Agar (TSA) plates to establish the numbers remaining.

### Conclusion of Assessment 6a

TABLE 2
Direct inoculation results
Bacterial numbers reduced to the detection limit (>5 log reduction)

| Microorganisms | 10 seconds | 1 minute | 2 minutes |
|---|---|---|---|
| E. coli | Yes | Yes | Yes |
| K. pneumoniae | Yes | Yes | Yes |
| E. faecalis | Yes | Yes | Yes |
| P. mirabilis | Yes | Yes | Yes |
| P. aeruginosa | Yes | Yes | Yes |

As such, Assessment 6a, and as shown in Table 2, demonstrates that a small volume of hypochlorous acid (250 ppm) solution is effective at reducing I×lO⁵ CFU/ml to the limit of detection, which is a greater than 5 log reduction for all time points (10 seconds, 1 minute, 2 minutes) against each challenge organism tested. Thus, Assessment 6a demonstrates that hypochlorous acid (250 ppm) solution is an effective and rapid antimicrobial against multiple, different strains of bacteria.

Moreover, Assessment 6a demonstrated that hypochlorous acid (250 ppm) solution has rapid antimicrobial activity against multiple CAUTI causing bacteria, with bacteria numbers being reduced to detection limit levels by 10 seconds.

### Assessment 6b—Antimicrobial Activity Along the Catheter Method

### Bacteria

Representative colonies from an 18-24 hour challenge culture plate were dispersed in maximum recovery diluent (MRD) to obtain an optical density (OD540 nm) equivalent to approximately I×lO⁸ CFU/ml. This was diluted to obtain a working concentration of I×lO⁴ CFU/ml. A quantitative count was performed to confirm the inoculum level.

The bacteria tested were Escherichia coli NCIMB 14067 which are bacteria that are commonly associated with causing CAUTI.

### Sample Preparation and Test Method

GentleCath Air 2.0 CH14 (manufactured by ConvaTec Inc.) intermittent urinary catheters were used for the assessment. Each catheter was prepared within a laminar flow cabinet to maintain sterility. In the assessment, each catheter was hydrated with a 100, 150, 200, 300, 675 or 1000 ppm hypochlorous acid solution. A further catheter was hydrated with sterile deionised water (SDW) and was used as a control.

Enough samples were prepared for n=3 and controls for n=1 per challenge organism.

Each catheter was hydrated with a hypochlorous solution as described above, as per the IFU, and removed from its packaging.

Each catheter was cut into three sections, each section having a length of 2.5 cm, beginning at the tip of the catheter (section 1) and ending proximal to the handle of the catheter (section 3). 10 pl of the I×lO⁴ CFU/ml challenge suspension was added to each catheter section, and each catheter section was then left undisturbed for two minutes to represent an approximate urination time.

Each catheter section was then placed into a sterile container alongside 10 ml of Dey-Engley Neutralising Broth (DENB) and sonicated for five minutes followed by
vortexing at 950 rpm for two minutes to allow any bacteria to detach from the surface of each catheter section.

Appropriate serial dilutions were performed and inoculated onto pre-dried TSA plates to establish the bacteria numbers remaining.

### Conclusion of Assessment 6b

The antimicrobial activity of GentleCath Air 2.0 CH14 intermittent urinary catheters hydrated with various hypochlorous acid concentration solutions is shown in FIG. 25. When the concentration of hypochlorous acid is at 300 ppm or above, the numbers of bacteria were consistently reduced to the detection limit, providing a greater than 93% bacterial cell death for each section of the catheter. Lower hypochlorous acid concentrations, i.e., 100, 150 and 200 ppm, also provided a good reduction in bacterial numbers with a bacteria kill rate of between 82 and 93% for catheter section 1, between 78 and 88% for catheter section 2, and between 50 and 93% for catheter section 3.

As such, assessment 6b demonstrated strong antimicrobial efficacy of hypochlorous acid solutions at various concentrations when used in conjunction with an intermittent urinary catheter. It is further advantageously noted that the strong antimicrobial efficacy of the hypochlorous acid solutions is present along each length of catheter tested, i.e., along a first section (tip of the catheter), a second section (middle portion of the length of the catheter) and a third section (proximal to the handle of the catheter). Thus, the testing showed that when used in conjunction with a catheter, hydrating the catheter with hypochlorous acid solutions in various concentrations results in antimicrobial activity present along the length of the catheter.

It is surprising to note that the catheter hydrated with sterile deionised water (SDW) used as a control displayed nearly 50% bacterial death in the test method. It is thought that the result of the control is likely due to the desiccation of the bacterial cells causing a reduction in bacteria numbers.

### Assessment 6c—Antimicrobial Residual Test

### Bacteria

Representative colonies from an 18-24 hour challenge culture plate were dispersed in maximum recovery diluent (MRD) to obtain an optical density (OD540 nm) equivalent to approximately I×lO⁸ CFU/ml. This was diluted to obtain a working concentration of I×lO⁶ CFU/ml. A quantitative count was performed to confirm the inoculum level.

The bacteria tested were Escherichia coli NCIMB 14067, Enterococcus faecalis NCTC 12201, which each are commonly associated with causing CAUTI.

### Sample Preparation and Test Method

40 pl of a hypochlorous acid solution (250 ppm), representative of the amount of hypochlorous acid that is left on a catheter following hydration, was placed onto sterile filters. A 40 pl volume of maximum recovery diluent (MRD) was placed onto a further sterile filter as a negative control. A 40 pl volume of approximate 2% Presept was placed onto a further sterile filter as a positive control. The filters used were Grade 427 mm Whatman Filters.

Following timepoints of 0 minutes, 30 minutes and 4 hours from adding the hypochlorous acid solutions and control to respective sterile filters, each sterile filter was then inoculated with lOpl of IxlO⁶ CFU/ml challenge suspension of bacteria, prepared as described above, and left undisturbed for two minutes (to represent approximate urination time).

Following the two minutes, the filters were homogenised in Dey-Engley Neutralising Broth (DENB) and appropriate serial dilutions were performed and inoculated onto pre-dried Tryptone Soy Agar (TSA) plates to establish the bacteria numbers remaining.

### Conclusion of Assessment 6c

As shown in Tables 3 and 4, similar results were obtained for both challenge organisms tested. The test method showed that the antimicrobial activity of hypochlorous acid solution (250 ppm) was exhausted by 30 minutes. This is observed by a ‘no growth’ of the challenge organisms at 0 hours (and an additional two minutes following inoculation of the solution, to represent approximate urination time), and then ‘growth’ of the challenge organisms at 30 minutes (plus two minutes).

Results for the negative control (MRD), where no antimicrobial activity is present, showed bacterial growth throughout the testing period. The positive control showed no bacterial growth throughout the testing period, which displays that even after four hours, the bleach solution (positive control) was still able to kill the inoculated bacteria.

As such, the antimicrobial residual test of Assessment 6c advantageously shows that the antimicrobial activity displayed by the hypochlorous acid solution (250 ppm) is diminished by 30 minutes after inoculation, therefore showing that hypochlorous acid does not accumulate in a patient's body following repeated use of the hypochlorous acid solution (as part of a lubricant for a catheter) throughout a period of time, for example a day.

TABLE 3
Antimicrobial residual test results for E. coli

| | 0 hour | 30 minutes | 4 hours |
|---|---|---|---|
| Solutions tested against E. | (plus 2 | (plus 2 | (plus 2 |
| coli NCIMB 14067 | minutes) | minutes) | minutes) |
| Hypochlorous acid (250 | No growth | Growth | Growth |
| ppm | | | |
| MRD (negative control) | Growth | Growth | Growth |
| Presept (bleach) control | No growth | No growth | No growth |
| (positive control) | | | |

TABLE 4
Antimicrobial residual test results for E. faecalis

| | 0 hour | 30 minutes | 4 hours |
|---|---|---|---|
| Solutions tested against E. | (plus 2 | (plus 2 | (plus 2 |
| faecalis NCTC 12201 | minutes) | minutes) | minutes) |
| Hypochlorous acid (250 | No growth | Growth | Growth |
| ppm) | | | |
| MRD (negative control) | Growth | Growth | Growth |
| Presept (bleach) control | No growth | No growth | No growth |
| (positive control) | | | |

### Conclusions

As such, the results show that hypochlorous acid solution demonstrate rapid antimicrobial activity against multiple catheter-associated urinary tract infection (CAUTI) causing bacteria, with detection limits reached by 10 seconds. The results further show that when used in conjunction with a catheter, the antimicrobial activity of hypochlorous acid was shown to be present along the length of the catheter. The antimicrobial residual testing provided further advantageous results, specifically demonstrating that the antimicrobial activity of hypochlorous acid is diminished by 30 minutes from hydration of a test sample.

It is clear that the invention significantly reduces the quantity of bacteria displaced along the urethra when in use. As such, the invention provides for highly effective and improved bacteria displacement performance which, in turn, will reduce the risk of a user developing a UTI. Moreover, it is clear that following initial, rapid antimicrobial activity against multiple

CAUTI causing bacteria, the activity of hypochlorous acid solution diminishes such that the hypochlorous acid compound breaks down and, therefore, advantageously, does not accumulate in the patient's body following repeated use of the hypochlorous acid solution (as part of a lubricant for a catheter) throughout a period of time, for example a day.

### Further Assessment 1

A further assessment was carried out using Lofric® Origo™ CH12 male intermittent urinary catheters (manufactured by Wellspect Limited). The Lofric® Origo™ CH 12 male intermittent urinary catheter comprises a polyvinyl pyrrolidone (PVP) surface coating. The catheter body is formed of a polyolefin-based elastomer (POBE).

The assessment included testing the bacterial displacement of a Lofric® Origo™ CH12 male intermittent urinary catheter hydrated with the lubricant of Example 5 (comprising an antimicrobial liquid comprising hypochlorous acid at a concentration of 0.1% wt./vol. (1000 ppm)), and the bacterial displacement of a Lofric® Origo™ CH12 male intermittent urinary catheter hydrated with the lubricant of Example 25 (comprising an antimicrobial liquid comprising chlorhexidine digluconate at a concentration of 0.06% wt./vol. (600 ppm)), against a Lofric® Origo™ CH12 male intermittent urinary catheter hydrated as per the IFU of which the lubricant did not contain an antimicrobial medium, and a positive control.

Preparation of the UACs, UAC inoculation, UAC processing and bacteria preparation was as described above in relation to Assessment 5.

The bacteria tested was Escherichia coli NCIMB 14067.

Improved bacterial displacement was visually observed with both catheters hydrated with the aforementioned antimicrobial mediums, compared to the catheter hydrated as per the IFU, which showed little to no bacterial displacement along the UAC.

Further, this observation was supported by TVC data which showed that undetectable levels of bacteria was achieved by section 3 of each respective UAC for both Lofric® Origo™ CH12 male intermittent urinary catheters hydrated with an antimicrobial medium.

Advantageously, this shows that lubricants of the invention provide a significant improvement to using Lofric® Origo™ CH 12 male intermittent urinary catheters compared to the same catheters hydrated as per the IFU, with regard to bacterial displacement testing.

### Further Assessment 2—Effect of Very Low Hypochlorous Acid Concentration on Sterilisation Potential

The antimicrobial efficacy of hypochlorous acid was tested at even lower concentrations (10, 25, and 50 ppm) against the challenging organism E. coli NCIMB 14067.

The representative colonies of E. coli NCIMB 14067 were dispersed in maximum recovery diluent (MRD) to obtain an optical density (OD540 nm) equivalent to approximately 1×10⁶ CFU/mL. A quantitative plate count was performed on this

### III

suspension to determine the total number of viable organisms inoculated. A new suspension was prepared for each concentration tested.

A 1000 ppm hypochlorous acid solution was diluted in sterile deionised water to provide the respective 10, 25 and 50 ppm hypochlorous acid solutions.

The hypochlorous acid samples were aliquoted into 9 mL volumes and 1 mL of the challenge organism suspension was added and mixed (the final number of bacteria present is now approximately 1×10⁶ CFU/mL). Following 5, 30 seconds, 1, 2, and 5 minutes, 100 pL volumes were sampled and transferred to 0.9 mL and 9.9 mL of Dey-Engley Neutralising Broth (DENB) (to provide a 1:10 and 1:100 dilution respectively), and total viable counts were performed to establish the amount present. The plates were left to dry before being inverted and incubated at 35+3° C. for at least 48 hours. A negative control was also tested whereby 1 mL of the challenge organism suspension was added to 9 mL of MRD and tested as previously described. Following the incubation period, the number of CFUs were counted on the most appropriate dilution for each sample i.e. between 25-250 CFU/plate.

The results from each concentration of hypochlorous acid solution against E. coli NCIMB 14067 showed that 25 and 50 ppm hypochlorous acid solutions resulted in undetectable numbers of bacteria after 30 seconds. The 10 ppm hypochlorous acid solution did show some minor bacterial recovery at 30 seconds (to a little over 10 CFU/mL), but this was reduced to undetectable levels at the 1 minute timepoint. Bacterial recovery for the negative control (MRD control) was maintained over the testing period, with CFU/mL being over four orders of magnitude larger than for the hypochlorous acid samples for all concentrations and time points tested.

Overall, despite the extremely low hypochlorous acid concentrations used, all solutions showed rapid antimicrobial activity against the challenging organism E. coli NCIMB 14067.

### Further Assessment 3—Antimicrobial Efficacy of Sulfanilamide (Comparative Compound—Not of the Invention)

For comparison purposes with the media of the invention, the sterilisation potential of a known antimicrobial agent was investigated.

An in vitro direct inoculation method was used to assess the antimicrobial activity of sulfanilamide in an intermittent urinary catheter scenario against grampositive Escherichia coli NCIMB 14067 and gram-negative Enterococcus faecalis NCTC 12201; two microorganisms associated with urinary tract infections. Sulfanilamide concentrations of 0.1% and 0.01% were tested and bacterial levels monitored over a period of 5 minutes.

Representative colonies of the challenge microorganisms, from an 18-24 hour challenge culture plate, were dispersed in MRD to obtain an optical density (OD540 nm) equivalent to approximately 1×10⁸ CFU/mL. This was diluted to obtain a working concentration of 1×10⁶ CFU/mL. A quantitative count was performed to confirm the inoculum level.

The challenge organism was prepared in MRD to a concentration of approximately 1×10⁶ CFU/mL. This suspension was added in a 1 mL volume to 9 mL of sample (n=3 per sample, n=2 per control) in a sterile 30 mL universal and pipetted up and down to mix. Following each testing period, 5 seconds, 30 seconds, 1 minute, 2 minutes and 5 minutes, 100 μL of the test solution was transferred to 0.9 mL and 9.9 mL volumes of DENB (1:10 and 1:100 dilutions, respectively). The most appropriate dilutions were inoculated (100 pL) onto duplicate pre-dried (tryptone soy agar) TSA plates and the inoculum spread using separate sterile L-shaped spreaders (for 10-1 and 10-2 dilutions, 0.5 mL were placed onto duplicate plates). The TSA plates were allowed to dry before being inverted and incubated at 35±3° C. for at least 48 hours. Following the incubation period, the number of bacterial colonies were counted on the most appropriate dilution for each dressing type i.e. between 25-250 CFU/plate.

The samples tested were: 0.1% sulfanilamide in sterile deionised water (SDW); 0.01% sulfanilamide in SDW; 100 ppm hypochlorous acid in SDW; and a SDW control.

There was no decrease in challenge organism numbers of E. coli NCIMB 14067 or E. faecalis NCTC 12201 when added to sulfanilamide (at both concentrations tested) when compared to the control for any of the timepoints tested. In contrast, when testing, 100 ppm hypochlorous acid solution resulted in undetectable numbers of both challenge organisms within the shortest timepoint of 5 seconds and these undetectable numbers were then observed for the remaining testing period.

### Conclusions (Further Assessments 1, 2 and 3)

As such, the results demonstrate excellent antimicrobial activity and sterilisation potential of a packaged intermittent urinary catheter of the invention. The tests have shown that the media of the invention facilitate simple and highly effective catheter cleaning methods. It is clear that the invention significantly reduces the quantity of bacteria displaced along the urethra when in use. Sterilisation can be achieved by as simply as contacting the catheter with the medium, such as by submerging the catheter in the medium. As such, the invention provides for highly effective and improved bacteria displacement performance which, in turn, will reduce the risk of a user developing a UTI. The one or more embodiments are described above by way of example only. Many variations are possible without departing from the scope of protection afforded by the appended claims.

1. A packaged intermittent urinary catheter comprising:
a single use catheter;
a packaging surrounding the catheter; and
a fluid reservoir holding a lubricant which, upon rupturing the fluid reservoir, is transferred into the packaging to immerse at least a portion of the catheter in the lubricant to prepare the catheter for use,
wherein the lubricant comprises an antimicrobial medium.

2. The packaged intermittent urinary catheter according to claim 1, wherein the antimicrobial medium comprises an aqueous chlorine solution.

3. The packaged intermittent urinary catheter according to claim 2, wherein the aqueous chlorine solution comprises at least one chlorine-containing species independently chosen from: hypochlorous acid, at least one hypochlorite salt, chlorine dioxide, at least one chlorhexidine salt, and combinations thereof.

4. The packaged intermittent urinary catheter according to claim 3, wherein the at least one chlorine-containing species is hypochlorous acid.

5. The packaged intermittent urinary catheter according to claim 3, wherein the at least one chlorine-containing species is present in an amount of between 100 and 4500 ppm of the overall aqueous content of the fluid reservoir.

6. The packaged intermittent urinary catheter according to claim 5, wherein the at least one chlorine-containing species is hypochlorous acid, and the hypochlorous acid is present in an amount of between 100 and 4500 ppm of the overall aqueous content of the fluid reservoir.

7. The packaged intermittent urinary catheter according to claim 3, wherein the total concentration of the at least one chlorine-containing species is of from 0.05 to 0.5% wt./vol of the overall aqueous content of the fluid reservoir.

8. The packaged intermittent urinary catheter according to claim 3, wherein the at least one chlorine-containing species is hypochlorous acid, and the total concentration of the hypochlorous acid is of from 0.05 to 0.5% wt./vol of the overall aqueous content of the fluid reservoir.

9. The packaged intermittent urinary catheter according to claim 3, wherein the total concentration of the at least one chlorine-containing species is no more than 0.45% wt./vol of the overall aqueous content of the fluid reservoir.

10. The packaged intermittent urinary catheter according to claim 3, wherein the at least one chlorine-containing species is hypochlorous acid, and the total concentration of the hypochlorous acid is no more than 0.45% wt./vol of the overall aqueous content of the fluid reservoir.

11. The packaged intermittent urinary catheter according to claim 1, wherein the antimicrobial medium does not comprise an alkali metal halide salt.

12. The packaged intermittent urinary catheter according to claim 3, wherein the at least one chlorine-containing species is hypochlorous acid, and the antimicrobial medium does not comprise an alkali metal halide salt.

13. The packaged intermittent urinary catheter according to claim 3, wherein the aqueous chlorine solution is obtained from a dichloroisocyanurate solution or a hypochlorite solution.

14. The packaged intermittent urinary catheter according to claim 3, wherein the at least one chlorhexidine salt is chlorhexidine digluconate, chlorhexidine diacetate, or combinations thereof.

15. The packaged intermittent urinary catheter according to claim 1, wherein the antimicrobial medium is present as an antimicrobial liquid or an antimicrobial gel.

16. The packaged intermittent urinary catheter according to claim 1, wherein the catheter is at least partially compounded with at least one additive.

17. The packaged intermittent urinary catheter according to claim 16, wherein the at least one additive is a lubricious additive, wherein the at least one additive is an amphiphilic additive.

18.-19. (canceled)

20. The packaged intermittent urinary catheter according to claim 1, wherein the fluid reservoir holds between 1 and 12 ml of lubricant.

21. (canceled)

22. A method of using a packaged intermittent urinary catheter including:
a. Providing a single use catheter in a sealed packaging surrounding the catheter;
b. Providing a fluid reservoir within the packaging, the fluid reservoir holding a lubricant comprising an antimicrobial medium;
c. Rupturing the fluid reservoir to transfer the lubricant into the packaging to immerse at least a portion of the catheter in the lubricant;
d. Forming an opening in the packaging; and
e. Removing the catheter from the packaging through the opening.

23. The method of using a packaged intermittent urinary catheter according to claim 22 comprising:
a. Removing a catheter from a packaging through an opening in the packaging;
b. Performing catheterisation with the catheter; and
c. Disposing of the catheter without re-use.

24-26: (canceled)
